(19) United States
(12) Patent Application Publication
(10) Pub. No.: US 2020/0172913 A1
(43) Pub. Date: Jun. 4, 2020

(54) PEPTIDES AND NANOPARTICLES FOR INTRACELLULAR DELIVERY OF VIRUS

(71) Applicant: Aadigen, LLC, Pacific Palisades, CA (US)

(72) Inventors: Neil DESAI, Pacific Palisades, CA (US); Gilles DIVITA, Saint Andre De Sangonis (FR)

(21) Appl. No.: 16/637,723

(22) PCT Filed: Aug. 9, 2018
(86) PCT No.: PCT/US2018/046138

§ 371 Date: Feb. 7, 2020

(30) Foreign Application Priority Data
Aug. 10, 2017 (FR) 17/57647

(51) Int. Cl. C12N 15/64; C07K 7/08; C12N 15/86; A61K 47/62; A61K 9/51; C12N 7/00

(52) U.S. Cl. CPC C12N 15/64; C07K 7/08; C12N 15/86; A61K 47/62; C12N 2750/14171; C12N 7/00; C12N 2750/14145; C12N 2750/14143; A61K 9/51

(57) ABSTRACT

The present invention pertains to peptide-containing complexes/nanoparticles that are useful for delivering into a cell one or more viruses (such as recombinant viruses, e.g., recombinant AAV) and/or masking antigenic epitopes on the one or more viruses.

### RELATED APPLICATION

The present application claims benefit of, and priority to French Application No. 1757647, filed on Aug. 10, 2017. The content of the French Application is incorporated herein by reference in its entirety.

### FIELD OF THE INVENTION

The present invention pertains to peptide-containing complexes/nanoparticles that are useful for delivering into a cell one or more viruses (such as recombinant viruses, e.g., recombinant AAV) and/or masking antigenic epitopes on the one or more viruses.

### BACKGROUND

The disclosures of all publications, patents, patent applications and published patent applications referred to herein are hereby incorporated herein by reference in their entirety.

### BRIEF SUMMARY OF THE INVENTION

The present application provides complexes and nanoparticles comprising cell-penetrating peptide that are useful for delivering into a cell one or more viruses (such as recombinant viruses, e.g., recombinant AAV) and/or masking antigenic epitopes on the one or more viruses. In some embodiments, the virus comprises a transgene, and intracellular delivery of the virus allows for transfer of the transgene into the cell genome. In some embodiments, the transgene encodes a protein, such as a therapeutic protein. In some embodiments, the transgene encodes an inhibitory RNA (RNAi), such as an RNAi targeting an endogenous gene, e.g., a disease-associated endogenous gene. In some embodiments, the transgene encodes a chimeric antigen receptor (CAR).

In some embodiments, there is provided a virus delivery complex for intracellular delivery of a virus comprising a cell-penetrating peptide associated with the virus.

In some embodiments, there is provided a virus delivery complex for intracellular delivery of a virus comprising a cell-penetrating peptide associated with the virus, wherein the cell-penetrating peptide is selected from the group consisting of PEP-1 peptides, PEP-2 peptides, PEP-3 peptides, VEPEP-3 peptides. VEPEP-6 peptides, VEPEP-9 peptides, and ADGN-100 peptides. In some embodiments, the virus is a recombinant virus including recombinant adeno-associated virus (AAV), adenovirus, lentivirus, retrovirus, herpes simplex virus (HSV), poxvirus, Epstein-Barr virus (EBV), vaccinia virus, and human cytomegalovirus (hCMV).

In some embodiments, according to any of the virus delivery complexes described above, the cell-penetrating peptide is a VEPEP-3 peptide. In some embodiments, the cell-penetrating peptide comprises an amino acid sequence selected from the group consisting of SEQ ID NOs: 1-14. In some embodiments, the cell-penetrating peptide comprises the amino acid sequence of SEQ ID NO: 75 or 76.

In some embodiments, the cell-penetrating peptide is a VEPEP-6 peptide. In some embodiments, the cell-penetrating peptide comprises an amino acid sequence selected from the group consisting of SEQ ID NOs: 15-40. In some embodiments, the cell-penetrating peptide comprises the amino acid sequence of SEQ ID NO: 77.

In some embodiments, according to any of the virus delivery complexes described above, the cell-penetrating peptide is a VEPEP-9 peptide. In some embodiments, the cell-penetrating peptide comprises an amino acid sequence selected from the group consisting of SEQ ID NOs: 41-52. In some embodiments, the cell-penetrating peptide comprises the amino acid sequence of SEQ ID NO: 78.

In some embodiments, according to any of the virus delivery complexes described above, the cell-penetrating peptide is an ADGN-100 peptide. In some embodiments, the cell-penetrating peptide comprises an amino acid sequence selected from the group consisting of SEQ ID NOs: 53-70. In some embodiments, the cell-penetrating peptide comprises the amino acid sequence of SEQ ID NO: 79 or 80.

In some embodiments, according to any of the virus delivery complexes described above, the cell-penetrating peptide further comprises one or more moieties covalently linked to the N-terminus of the cell-penetrating peptide, and wherein the one or more moieties are selected from the group consisting of an acetyl, a fatty acid, a cholesterol, a poly-ethylene glycol, a nuclear localization signal, nuclear export signal, an antibody, a polysaccharide and a targeting molecule. In some embodiments, the cell-penetrating peptide comprises an acetyl group covalently linked to its N-terminus. In some embodiments, the cell-penetrating peptide further comprises one or more moieties covalently linked to the C-terminus of the cell-penetrating peptide, and wherein the one or more moieties are selected from the group consisting of a cysteamide, a cysteine, a thiol, an amide, a nitrilotriacetic acid optionally substituted, a carboxyl, a linear or ramified C₁-C₆ alkyl optionally substituted, a primary or secondary amine, an osidic derivative, a lipid, a phospholipid, a fatty acid, a cholesterol, a poly-ethylene glycol, a nuclear localization signal, nuclear export signal, an antibody, a polysaccharide and a targeting molecule. In some embodiments, the cell-penetrating peptide comprises a cysteamide group covalently linked to its C-terminus.

In some embodiments, according to any of the virus delivery complexes described above, at least some of the cell-penetrating peptides in the virus delivery complex are linked to a targeting moiety by a linkage. In some embodiments, the linkage is covalent.

In some embodiments, according to any of the virus delivery complexes described above, the molar ratio of the cell-penetrating peptide to the virus (e.g., in Vg, pfu, or MOI) is between about 1:1 and about 1×10⁸:1.

In some embodiments, according to any of the virus delivery complexes described above, the average diameter of the virus delivery complex is between about 20 nm and about 1000 nm.

In some embodiments, there is provided a nanoparticle comprising a core comprising a virus delivery complex according to any of the embodiments described above. In some embodiments, the core further comprises one or more additional virus delivery complexes according to any of the embodiments described above. In some embodiments, at least some of the cell-penetrating peptides in the nanoparticle are linked to a targeting moiety by a linkage. In some embodiments, the core is coated by a shell comprising peripheral cell-penetrating peptides. In some embodiments, at least some of the peripheral cell-penetrating peptides in the shell are linked to a targeting moiety by a linkage. In some embodiments, the linkage is covalent. In some embodiments, the peripheral cell-penetrating peptide is selected from the group consisting of PEP-1 peptides, PEP-2 peptides, PEP-3 peptides, VEPEP-3 peptides, VEPEP-6 peptides, VEPEP-9 peptides, and ADGN-100 peptides. In some embodiments, the peripheral cell-penetrating peptide comprises an amino acid sequence selected from the group consisting of SEQ ID NOs: 1-80. In some embodiments, the average diameter of the nanoparticle is between about 20 nm and about 1000 nm. In some embodiments, the average diameter of the nanoparticle is between about 50 nm and about 800 nm. In some embodiments, the average diameter of the nanoparticle is between about 100 nm and about 500 nm.

In some embodiments, there is provided a pharmaceutical composition comprising a virus delivery complex according to any of the embodiments described above or a nanoparticle according to any of the embodiments described above, and a pharmaceutically acceptable carrier. In some embodiments, the pharmaceutical composition is formulated for intravenous, intratumoral, intraarterial, topical, intraocular, ophthalmic, intraportal, intracranial, intracerebral, intracerebroventricular, intrathecal, intravesicular, intradermal, subcutaneous, intramuscular, intranasal, intratracheal, pulmonary, intracavity, or oral administration. In some embodiments, the pharmaceutical composition is lyophilized.

In some embodiments, there is provided a method of preparing a virus delivery complex according to any of the embodiments described above, comprising combining the cell-penetrating peptide with the virus, thereby forming the virus delivery complex. In some embodiments, the cell-penetrating peptide is combined with the virus (e.g., in Vg, pfu, or MOI) at a ratio from about 1:1 to about 1×10⁸: 1, respectively.

In some embodiments, there is provided a method of delivering one or more viruses into a cell, comprising contacting the cell with a virus delivery complex according to any of the embodiments described above and/or a nanoparticle according to any of the embodiments described above, wherein the virus delivery complex and/or the nanoparticle comprises the one or more viruses. In some embodiments, the contacting of the cell with the virus delivery complex and/or nanoparticle is carried out in vivo. In some embodiments, the contacting of the cell with the virus delivery complex and/or nanoparticle is carried out ex vivo. In some embodiments, the contacting of the cell with the virus delivery complex and/or nanoparticle is carried out in vitro. In some embodiments, the cell is a granulocyte, a mast cell, a monocyte, a dendritic cell, a B cell, a T cell, a natural killer cell, a fibroblast, a muscle cell, a cardiac cell, or a hepatocyte. In some embodiments, the cell is a T cell. In some embodiments, the cell is a fibroblast. In some embodiments, the cell is a hepatocyte. In some embodiments, the cell is a lung progenitor cell. In some embodiments, the cell is a neuronal cell. In some embodiments, the virus targets a sequence in a gene selected from the group consisting of PD-1, PD-L1, PD-L2, TIM-3, BTLA, VISTA, LAG-3, CTLA-4, TIGIT, 4-1BB, OX40, CD27, TIM-1, CD28, HVEM, GITR, and ICOS. In some embodiments, the virus comprises a nucleic acid molecule encoding an exogenous protein. In some embodiments, the exogenous protein is a recombinant receptor capable of being expressed on the surface of a cell. In some embodiments, the recombinant receptor is a chimeric antigen receptor (CAR).

In some embodiments, there is provided a method of intracellular delivery of a virus in a cell, comprising contacting the cell with a virus delivery complex according to any of the embodiments described above or a nanoparticle according to any of the embodiments described above, wherein the virus delivery complex or the nanoparticle comprises one or more viruses.

In some embodiments, there is provided a method of treating a disease in an individual comprising administering to the individual an effective amount of a pharmaceutical composition according to any of the embodiments described above. In some embodiments, the disease is selected from the group consisting of cancer, diabetes, autoimmune diseases, inflammatory diseases, fibrotic diseases, viral infectious diseases, hereditary diseases, ocular diseases, liver diseases, lung diseases, muscle diseases, enzyme deficiency diseases, lysosomal storage diseases, neurological diseases, kidney diseases, and aging and degenerative diseases. In some embodiments, the pharmaceutical composition is used for gene therapy in the individual.

In some embodiments, the disease is cancer. In some embodiments, the cancer is a solid tumor, and the pharmaceutical composition comprises a virus delivery complex or nanoparticle comprising one or more virus that modulates the expression of one or more proteins selected from the group consisting of growth factors and cytokines, cell surface receptors, signaling molecules and kinases, transcription factors and other modulators of transcription, regulators of protein expression and modification, and regulators of apoptosis and metastasis. In some embodiments, the cancer is cancer of the liver, lung, or kidney. In some embodiments, the cancer is a hematological malignancy, and the pharmaceutical composition comprises a virus delivery complex or nanoparticle comprising one or more viruses that modulates the expression of one or more proteins selected from the group consisting of growth factors and cytokines, cell surface receptors, signaling molecules and kinases, transcription factors and other modulators of transcription, regulators of protein expression and modification, and regulators of apoptosis and metastasis.

In some embodiments, according to any of the methods of treating a disease described above, the disease is a viral infection disease, and the pharmaceutical composition comprises a virus delivery complex or nanoparticle comprising one or more viruses that modulates the expression of one or more proteins involved in the viral infectious disease development and/or progression.

In some embodiments, according to any of the methods of treating a disease described above, the disease is a genetic disease, and the pharmaceutical composition comprises a virus delivery complex or nanoparticle comprising one or more viruses that modulates the expression of one or more proteins involved in the hereditary disease development and/or progression.

In some embodiments, according to any of the methods of treating a disease described above, the disease is an aging or degenerative disease, and the pharmaceutical composition comprises a virus delivery complex or nanoparticle comprising one or more viruses that modulates the expression of one or more proteins involved in the aging or degenerative disease development and/or progression.

In some embodiments, according to any of the methods of treating a disease described above, the disease is a fibrotic or inflammatory disease, and the pharmaceutical composition comprises a virus delivery complex or nanoparticle comprising one or more viruses that modulates the expression of two or more proteins involved in the fibrotic or inflammatory disease development and/or progression.

In some embodiments, according to any of the methods of treating a disease described above, the pharmaceutical composition comprising a virus delivery complex or nanoparticle is less immunogenic than a similar pharmaceutical composition comprising the one or more viruses contained in the virus delivery complex or nanoparticle alone (i.e., a pharmaceutical composition comprising the one or more viruses not associated with a peptide as described herein).

In some embodiments, according to any of the methods of treating a disease described above, the method comprises multiple administrations of the pharmaceutical composition comprising a virus delivery complex or nanoparticle. In some embodiments, repeated administrations of the pharmaceutical compositions do not elicit an adverse immune response in the individual to the pharmaceutical composition, or elicit a substantially reduced immune response in the individual compared to repeated administrations of a similar pharmaceutical composition comprising the one or more viruses contained in the virus delivery complex or nanoparticle alone.

In some embodiments, according to any of the methods of treating a disease described above, the individual produces neutralizing antibodies to at least one of the one or more viruses contained in a virus delivery complex or nanoparticle in the pharmaceutical composition, and the peptides of the virus delivery complex or nanoparticle mask the at least one virus from the neutralizing antibodies. In some embodiments, the neutralizing antibodies are blocked from neutralizing the at least one virus, or result in substantially reduced neutralizing of the at least one virus compared to the at least one virus alone (i.e., the at least one virus not associated with a peptide as described herein).

In some embodiments, according to any of the methods of treating a disease described above, the individual is human.

In some embodiments, there is provided a kit comprising a composition comprising a virus delivery complex according to any of the embodiments described above and/or a nanoparticle according to any of the embodiments described above.

### BRIEF DESCRIPTION OF THE FIGURES

FIGS. 1A and 1B show that association of CPP with AAV-2 promotes viral transduction in HepG2 (FIG. 1A) and HS68 (FIG. 1B) cells. Cells were infected with AAV-2-GFP (MOI of 400) alone or pre-complexed with PEP1, PEP2, or P-ANT at 0.1 μM, 1 μM, 10 μM, 100 μM, 200 μM, and 500 μM. The percentage of GFP-expressing cells was detected by flow cytometry.

FIG. 2 shows that association of CPP with AAV-6 promotes viral transduction in HUVEC cells. Cells were infected with AAV-6 expressing β-galactosidase (MOI of 400) alone or pre-complexed with PEP1, PEP2, or P-ANT at 0.1 μM, 1 μM, 10 μM, 100 μM, 200 μM and 500 μM. The percentage of β-galactosidase activity was determined in cell lysates.

FIG. 3 shows the toxicity profile of the CPPs in HUVEC cells. Adenoviruses encoding β-galacosidase (AAV-βGal) were pre-incubated with PEP-1, PEP-2, and Penetratin at concentration ranging from 1 μM to 500 μM. Cells cytotoxicity of AAV-CPP complexes was determined using the XTT assay after 2 days.

FIGS. 4A and 4B show that CPPs influence the titer necessary for virus-mediated gene expression. A fixed 200 μM concentration of peptides (PEP-1, PEP-2, Penetratin, and TAT) were pre-incubated with increasing MOI (up to 2000) of AAV-2 encoding green fluorescent protein (AAV-GFP). HS 68 (FIG. 4B) and HepG2 (FIG. 4A) cells were treated with either free AAV-2 or AAV/CPP complexes for 4 h. GFP expression was analyzed 2 days after infection.

FIGS. 5A-5D show that association of CPP with AAV-1 promotes viral transduction in HepG2 (FIGS. 5A and 5B) and HCN2 (FIGS. 5C and 5D) cells. Cells were infected with AAV-1-GFP (MOI of 500) alone or pre-complexed with ADGN-103a, ADGN-103b, ADGN-100, ADGN-109, ADGN-106, PEP-1, PEP-2, CADY, or TAT-HA2 at 0.1 μM, 1 μM, 10 μM, 100 μM and 200 μM. The percentage of GFP expressing cells was detected by flow cytometry (FIGS. 5A and 5C) and the increase in AAV-1 infectivity was calculated based on the number of GFP positive cells (FIGS. 5B and 5D).

FIGS. 6A-6D show that association of CPP with AAV-2 promotes viral transduction in HepG2 (FIGS. 6A and 6B) and HCN2 (FIGS. 6C and 6D) cells. Cells were infected with AAV-2-GFP (MOI of 500) alone or pre-complexed with ADGN-103a, ADGN-103b, ADGN-100, ADGN-109, ADGN-106, PEP-1, PEP-2, CADY, or TAT-HA2 at 0.1 μM, 1 μM, 10 μM, 100 μM and 200 μM. The percentage of GFP expressing cells was detected by flow cytometry (FIGS. 6A and 6C) and the increase in AAV-2 infectivity was calculated based on the number of GFP positive cells (FIGS. 6B and 6D).

FIGS. 7A-7D show that association of CPP with AAV-5 promotes viral transduction in HepG2 (FIGS. 7A and 7B) and HCN2 (FIGS. 7C and 7D) cells. Cells were infected with AAV-5-GFP (MOI of 500) alone or pre-complexed with ADGN-103a, ADGN-103b, ADGN-100, ADGN-109, ADGN-106, PEP-1, PEP-2, CADY, or TAT-HA2 at 0.1 μM, 1 μM, 10 μM, 100 μM and 200 μM. The percentage of GFP expressing cells was detected by flow cytometry (FIGS. 7A and 7C) and the increase in AAV-5 infectivity was calculated based on the number of GFP positive cells (FIGS. 7B and 7D).

FIGS. 8A-8D show that association of CPP with AAV-6 promotes viral transduction in HepG2 (FIGS. 8A and 8B) and HCN2 (FIGS. 8C and 8D) cells. Cells were infected with AAV-6-GFP (MOI of 500) alone or pre-complexed with ADGN-103a, ADGN-103b, ADGN-100, ADGN-109, ADGN-106, PEP-1, PEP-2, CADY, or TAT-HA2 at 0.1 μM, 1 μM, 10 μM, 100 μM and 200 μM. The percentage of GFP expressing cells was detected by flow cytometry (FIGS. 8A and 8C) and the increase in AAV-6 infectivity was calculated based on the number of GFP positive cells (FIGS. 8B and 8D).

FIGS. 9A-9D show that association of CPP with AAV-8 promotes viral transduction in HepG2 (FIGS. 9A and 9B) and HCN2 (FIGS. 9C and 9D) cells. Cells were infected with AAV-8-GFP (MOI of 1000) alone or pre-complexed with ADGN-103a, ADGN-103b, ADGN-100, ADGN-109, ADGN-106, PEP-1, PEP-2, CADY, or TAT-HA2 at 0.1 μM, 1 μM, 10 μM, 100 μM and 200 μM. The percentage of GFP expressing cells was detected by flow cytometry (FIGS. 9A and 9C) and the increase in AAV-8 infectivity was calculated based on the number of GFP positive cells (FIGS. 9B and 9D).

FIG. 10 shows the modification of CPP, such as a targeting molecule conjugated to the N-terminus or C-terminus of the CPP (CPP-T or T-CPP), a Dopamine moiety conjugated to the N-terminus of the CPP (Dop-CPP) or a PEG moiety conjugated to the N-terminus of the CPP (PEG-CPP) promoted viral transduction in cells.

### DETAILED DESCRIPTION OF THE INVENTION

In order for virally-mediated genome-editing techniques to be therapeutically applicable, the virus must be safely and efficiently delivered inside of target cells, such as disease cells of a target disease. Adeno-associated viral particles have commonly been used as gene delivery agents, however their clinical use has been limited due to safety issues arising from infection of off-target cells and immunogenicity. Thus, there is a need for improved methods for safe and efficient delivery of virus inside target cells.

The present application provides complexes and nanoparticles comprising a cell-penetrating peptide (CPP) and one or more viruses, wherein the CPP is suitable for delivering into a cell the one or more viruses (such as recombinant viruses, e.g., recombinant AAV) and/or masking antigenic epitopes on the one or more viruses. The complexes and nanoparticles may comprise a plurality of viruses. The viruses may include, for example, recombinant AAV, adenovirus, lentivirus, retrovirus. HSV, poxvirus, EBV, vaccinia virus, and hCMV.

Thus, the present application in one aspect provides novel virus delivery complexes and nanoparticles which are described further below in more detail.

In another aspect, there are provided methods of delivering a virus into a cell using the cell-penetrating peptides.

Also provided are pharmaceutical compositions comprising a cell-penetrating peptide and one or more viruses (for example in the forms of complexes and nanoparticles) and uses thereof for treating diseases.

### Definitions

As used herein the term “wild type” is a term of the art understood by skilled persons and means the typical form of an organism, strain, gene or characteristic as it occurs in nature as distinguished from mutant or variant forms.

As used herein the term “variant” should be taken to mean the exhibition of qualities that have a pattern that deviates from what occurs in nature.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

“Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein. “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating” refers to an approach for obtaining beneficial or desired results including but not limited to a therapeutic benefit. By therapeutic benefit is meant any therapeutically relevant improvement in or effect on one or more diseases, conditions, or symptoms under treatment.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

As used herein, the singular form “a”, “an”, and “the” includes plural references unless indicated otherwise.

Reference to “about” a value or parameter herein includes (and describes) embodiments that are directed to that value or parameter per se. For example, description referring to “about X” includes description of “X.”

The compositions and methods of the present invention may comprise, consist of, or consist essentially of the essential elements and limitations of the invention described herein, as well as any additional or optional ingredients, components, or limitations described herein or otherwise useful.

Unless otherwise noted, technical terms are used according to conventional usage.

### Complexes and Nanoparticles

In some aspects, the invention provides complexes and nanoparticles comprising cell-penetrating peptides for delivering one or more viruses into a cell. In some embodiments, cell-penetrating peptides are complexed with the one or more viruses. In some embodiments, the cell-penetrating peptides are non-covalently complexed with at least one of the one or more viruses. In some embodiments, the cell-penetrating peptides are non-covalently complexed with each of the one or more viruses. In some embodiments, the cell-penetrating peptides are covalently complexed with at least one of the one or more viruses. In some embodiments, the cell-penetrating peptides are covalently complexed with each of the one or more viruses. In some embodiments, the virus is a recombinant virus, including recombinant AAV, adenovirus, lentivirus, retrovirus, HSV, poxvirus, EBV, vaccinia virus, and hCMV. In some embodiments, the recombinant virus comprises a transgene, and intracellular delivery of the virus allows for transfer of the transgene into the genome of the cell. In some embodiments, the transgene is a therapeutic transgene. In some embodiments, the transgene encodes a protein, such as a therapeutic protein. In some embodiments, the transgene encodes an inhibitory RNA (RNAi), such as an RNAi targeting an endogenous gene, e.g., a disease-associated endogenous gene. In some embodiments, the transgene encodes a CAR. In some embodiments, the complex and/or nanoparticle comprises one or more viruses comprising a first transgene encoding an RNAi and a second transgene encoding a protein. In some embodiments, the RNAi is a therapeutic RNAi targeting an endogenous gene involved in a disease or condition, and the protein is a therapeutic protein useful for treating the disease or condition. In some embodiments, the therapeutic RNAi targets a disease-associated form of the endogenous gene (e.g., a gene encoding a mutant protein, or a gene resulting in abnormal expression of a protein), and the second transgene is a therapeutic form of the endogenous gene (e.g., the second transgene encodes a wild-type or functional form of the mutant protein, or the second transgene results in normal expression of the protein). For example, in some embodiments, the complex and/or nanoparticle comprises a first transgene encoding an RNAi targeting mutant and normal endogenous rhodopsin genes, and the second transgene comprises a function rhodopsin gene that is resistant to the RNAi. In some embodiments, the complex and/or nanoparticle comprises a first virus comprising the first transgene and a second virus comprising the second transgene. In some embodiments, the complex and/or nanoparticle comprises a single virus comprising the first transgene and the second transgene.

### Cell-Penetrating Peptides

The cell-penetrating peptides in the virus delivery complexes or nanoparticles of the present invention are capable of forming stable complexes and nanoparticles with various viruses. Any of the cell-penetrating peptides in any of the virus delivery complexes or nanoparticles described herein may comprise or consist of any of the cell-penetrating peptide sequences described in this section.

In some embodiments, a virus delivery complex or nanoparticle described herein comprises a cell-penetrating peptide selected from the group consisting of CADY. PEP-1, PEP-2, MPG, VEPEP-3 peptides, VEPEP-4 peptides, VEPEP-5 peptides, VEPEP-6 peptides, VEPEP-9 peptides, and ADGN-100 peptides. In some embodiments, the cell-penetrating peptide is present in a virus delivery complex. In some embodiments, the cell-penetrating peptide is present in a virus delivery complex present in the core of a nanoparticle. In some embodiments, the cell-penetrating peptide is present in the core of a nanoparticle. In some embodiments, the cell-penetrating peptide is present in the core of a nanoparticle and is associated with a virus. In some embodiments, the cell-penetrating peptide is present in an intermediate layer of a nanoparticle. In some embodiments, the cell-penetrating peptide is present in the surface layer of a nanoparticle. In some embodiments, the cell-penetrating peptide is linked to a targeting moiety. In some embodiments, the linkage is covalent. In some embodiments, the covalent linkage is by chemical coupling. In some embodiments, the covalent linkage is by genetic methods. WO2014/053879 discloses VEPEP-3 peptides: WO2014/053881 discloses VEPEP-4 peptides; WO2014/053882 discloses VEPEP-5 peptides; WO2012/137150 discloses VEPEP-6 peptides; WO2014/053880 discloses VEPEP-9 peptides; WO 2016/102687 discloses ADGN-100 peptides; US2010/0099626 discloses CADY peptides; and U.S. Pat. No. 7,514,530 discloses MPG peptides; the disclosures of which are hereby incorporated herein by reference in their entirety.

In some embodiments, a virus delivery complex or nanoparticle described herein comprises a VEPEP-3 cell-penetrating peptide comprising the amino acid sequence X₁X₂X₃X₄X₅X₂X₃X₄X₆X₇X₃X₈X₉X₁₀X₁₁X₁₂X₁₃ (SEQ ID NO: 1), wherein X₁ is beta-A or S, X₂ is K. R or L (independently from each other), X₃ is F or W (independently from each other), X₄ is F, W or Y (independently from each other), X₅ is E, R or S, X₆ is R, T or S, X₇ is E, R, or S, X₈ is none, F or W, X₉ is P or R, X₁₀ is R or L, X₁₁ is K, W or R, X₁₂ is R or F, and X₁₃ is R or K. In some embodiments, the VEPEP-3 peptide comprises the amino acid sequence X₁X₂WX₄EX₂WX₄X₆X₇X₃PRX₁₁RX₁₃ (SEQ ID NO: 2), wherein X₁ is beta-A or S, X₂ is K, R or L, X₃ is F or W, X₄ is F, W or Y, X₅ is E, R or S, X₆ is R, T or S, X₇ is E, R, or S, X₈ is none, F or W, X₉ is P or R, X₁₀ is R or L, X₁₁ is K, W or R, X₁₂ is R or F, and X₁₃ is R or K. In some embodiments, the VEPEP-3 peptide comprises the amino acid sequence X₁KWFERWFREWPRKRR (SEQ ID NO: 3), X₁KWWERWWREWPRKRR (SEQ ID NO: 4), X₁KWWERWWREWPRKRK (SEQ ID NO: 5). X₁RWWEKWWTRWPRKRK (SEQ ID NO: 6), or X₁RWYEKWYTEFPRRRR (SEQ ID NO: 7), wherein X₁ is beta-A or S. In some embodiments, the VEPEP-3 peptide comprises the amino acid sequence of any one of SEQ ID NOs: 1-7, wherein the cell-penetrating peptide is modified by replacement of the amino acid in position 10 by a non-natural amino acid, addition of a non-natural amino acid between the amino acids in positions 2 and 3, and addition of a hydrocarbon linkage between the two non-natural amino acids. In some embodiments, the VEPEP-3 peptide comprises the amino acid sequence X₁KX₁₄WWERWWRX₁₄WPRKRK (SEQ ID NO: 8), wherein X₁ is beta-A or S and X₁₄ is a non-natural amino acid, and wherein there is a hydrocarbon linkage between the two non-natural amino acids. In some embodiments, the VEPEP-3 peptide comprises the amino acid sequence X₁X₂X₃WX₅X₁₀X₃WX₆X₇WX₈X₉X₁₀WX₁₂R (SEQ ID NO: 9), wherein X₁ is beta-A or S, X, is K, R or L, X₃ is F or W, X₅ is R or S, X₆ is R or S, X₇ is R or S, X₈ is F or W, X₉ is R or P, X₁₀ is L or R, and X₁₂ is R or F. In some embodiments, the VEPEP-3 peptide comprises the amino acid sequence X₁RWWRLWWRSWFRLWRR (SEQ ID NO: 10), X₁LWWRRWWSRWWPRWRR (SEQ ID NO: 11), X₁LWWSRWWRSWFRLWFR (SEQ ID NO: 12), or X₁KFWSRFWRSWFRLWRR (SEQ ID NO: 13), wherein X₁ is beta-A or S. In some embodiments, the VEPEP-3 peptide comprises the amino acid sequence of any one of SEQ ID NOs: 1 and 9-13, wherein the cell-penetrating peptide is modified by replacement of the amino acids in position 5 and 12 by non-natural amino acids, and addition of a hydrocarbon linkage between the two non-natural amino acids. In some embodiments, the VEPEP-3 peptide comprises the amino acid sequence X₁RWWX₁₄LWWRSWX₁₄RLWRR (SEQ ID NO: 14), wherein X₁ is a beta-alanine or a serine and X₁₄ is a non-natural amino acid, and wherein there is a hydrocarbon linkage between the two non-natural amino acids. In some embodiments, the VEPEP-3 peptide is present in a virus delivery complex. In some embodiments, the VEPEP-3 peptide is present in a virus delivery complex in the core of a nanoparticle. In some embodiments, the VEPEP-3 peptide is present in the core of a nanoparticle. In some embodiments, the VEPEP-3 peptide is present in the core of a nanoparticle and is associated with a virus. In some embodiments, the VEPEP-3 peptide is present in an intermediate layer of a nanoparticle. In some embodiments, the VEPEP-3 peptide is present in the surface layer of a nanoparticle. In some embodiments, the VEPEP-3 peptide is linked to a targeting moiety. In some embodiments, the linkage is covalent. In some embodiments, the covalent linkage is by chemical coupling. In some embodiments, the covalent linkage is by genetic methods.

In some embodiments, a virus delivery complex or nanoparticle described herein comprises a VEPEP-6 cell-penetrating peptide. In some embodiments, the VEPEP-6 peptide comprises an amino acid sequence selected from the group consisting of X₁LX₂RALWX₉LX₃X₉X₄LWX₉LX₅X₆X₇X₈(SEQ ID NO: 15), X₁LX₂LARWX₉LX₃X₉X₄LWX₉LX₅X₆X₇X₈ (SEQ ID NO: 16) and X₁LX₂ARLWX₉LX₃X₉X₄LWX₉LX₅X₆X₇X₈ (SEQ ID NO: 17), wherein X₁ is beta-A or S, X₂ is F or W, X₁ is L, W, C or I, X₄ is S, A, N or T, X₅ is L or W, X₆ is W or R, X₇ is K or R, X₈ is A or none, and X₉ is R or S. In some embodiments, the VEPEP-6 peptide comprises the amino acid sequence X₁LX₂RALWRLX₃RX₄LWRLX₅X₆X₇X₈(SEQ ID NO: 18), wherein X₁ is beta-A or S, X₂ is F or W, X₃ is L, W, C or I, X₄ is S, A, N or T, X₅ is L or W, X₆ is W or R, X₇ is K or R, and X₈ is A or none. In some embodiments, the VEPEP-6 peptide comprises the amino acid sequence X₁LX₂RALWRLX₃RX₄LWRLX₅X₆KX₇ (SEQ ID NO: 19), wherein X₁ is beta-A or S, X₂ is F or W, X₃ is L or W, X₄ is S, A or N, X₅ is L or W, X₆ is W or R, X₇ is A or none. In some embodiments, the VEPEP-6 peptide comprises an amino acid sequence selected from the group consisting of X₁LFRALWRLLRX₂LWRLLWX₃ (SEQ ID NO: 20), X₁LWRALWRLWRX₂LWRLLWX₃A (SEQ ID NO: 21), X₁LWRALWRLX₄RX₂LWRLWRX₃A (SEQ ID NO: 22), X₁LWRALWRLWRX₂LWRLWRX₃A (SEQ ID NO: 23), X₁LWRALWRLX₅RALWRLLWX₃A (SEQ ID NO: 24), and X₁LWRALWRLX₄RNLWRLLWX₃A (SEQ ID NO: 25), wherein X₁ is beta-A or S, X₂ is S or T, X₃ is K or R, X₄ is L, C or I and X₅ is L or I. In some embodiments, the VEPEP-6 peptide comprises an amino acid sequence selected from the group consisting of Ac-X₁LFRALWRLLRSLWRLLWK-cysteamide (SEQ ID NO: 26), Ac-X₁LWRALWRLWRSLWRLLWKA-cysteamide (SEQ ID NO: 27), Ac-X₁LWRALWRLLRSLWRLWRKA-cysteamide (SEQ ID NO: 28), Ac-X₁LWRALWRLWRSLWRLWRKA-cysteamide (SEQ ID NO: 29), Ac-X₁LWRALWRLLRALWRLLWKA-cysteamide (SEQ ID NO: 30), and Ac-X₁LWRALWRLLRNLWRLLWKA-cysteamide (SEQ ID NO: 31), wherein X₁ is beta-A or S. In some embodiments, the VEPEP-6 peptide comprises the amino acid sequence of any one of SEQ ID NOs: 15-31, further comprising a hydrocarbon linkage between two residues at positions 8 and 12. In some embodiments, the VEPEP-6 peptide comprises an amino acid sequence selected from the group consisting of Ac-X₁LFRALWR_{S}LLRS_{S}LWRLLWK-cysteamide (SEQ ID NO: 32), Ac-X₁LFLARWR_{S}LLRS_{S}LWRLLWK-cysteamide (SEQ ID NO: 33), Ac-X₁LFRALWS_{S}LLRS_{S}LWRLLWK-cysteamide (SEQ ID NO: 34), Ac-X₁LFLARWS_{S}LLRS_{S}LWRLLWK-cysteamide (SEQ ID NO: 35), Ac-X₁LFRALWRLLR_{S}SLWS_{S}LLWK-cysteamide (SEQ ID NO: 36), Ac-X₁LFLARWRLLR_{S}SLWS_{S}LLWK-cysteamide (SEQ ID NO: 37), Ac-X₁LFRALWRLLS_{S}SLWS_{S}LLWK-cysteamide (SEQ ID NO: 38), Ac-X₁LFLARWRLLS_{S}SLWS_{S}LLWK-cysteamide (SEQ ID NO: 39), and Ac-X₁LFAR_{S}LWRLLRS_{S}LWRLLWK-cysteamide (SEQ ID NO: 40), wherein X₁ is beta-A or S and wherein the residues followed by an inferior “S” are those which are linked by said hydrocarbon linkage. In some embodiments, the VEPEP-6 peptide is present in a virus delivery complex. In some embodiments, the VEPEP-6 peptide is present in a virus delivery complex in the core of a nanoparticle. In some embodiments, the VEPEP-6 peptide is present in the core of a nanoparticle. In some embodiments, the VEPEP-6 peptide is present in the core of a nanoparticle and is associated with a virus. In some embodiments, the VEPEP-6 peptide is present in an intermediate layer of a nanoparticle. In some embodiments, the VEPEP-6 peptide is present in the surface layer of a nanoparticle. In some embodiments, the VEPEP-6 peptide is linked to a targeting moiety. In some embodiments, the linkage is covalent. In some embodiments, the covalent linkage is by chemical coupling. In some embodiments, the covalent linkage is by genetic methods.

In some embodiments, a virus delivery complex or nanoparticle described herein comprises a VEPEP-9 cell-penetrating peptide comprising the amino acid sequence X₁X₂X₃WWX₄X₅WAX₆X₃X₇X₈X₉X₁₀X₁₁X₁₂WX₁₃R (SEQ ID NO: 41), wherein X₁ is beta-A or S, X₂ is L or none, X₃ is R or none, X₄ is L, R or G, X₅ is R, W or S, X₆ is S, P or T, X, is W or P, X₈ is F, A or R, X₉ is S, L, P or R, X₁₀ is R or S, X₁₁ is W or none, X₁₂ is A, R or none and X₁₃ is W or F, and wherein if X₃ is none, then X₂, X₁₁ and X₁₂ are none as well. In some embodiments, the VEPEP-9 peptide comprises the amino acid sequence X₁X₂RWWLRWAX₆RWX₈X₉X₁₀WX₁₂WX₁₃R (SEQ ID NO: 42), wherein X₁ is beta-A or S, X₂ is L or none, X₆ is S or P, X₈ is F or A, X₉ is S, L or P, X₁₀ is R or S, X₁₂ is A or R, and X₁₃ is W or F. In some embodiments, the VEPEP-9 peptide comprises an amino acid sequence selected from the group consisting of X₁LRWWLRWASRWFSRWAWWR (SEQ ID NO: 43), X₁LRWWLRWASRWASRWAWFR (SEQ ID NO: 44), X₁RWWLRWASRWALSWRWWR (SEQ ID NO: 45), X₁RWWLRWASRWFLSWRWWR (SEQ ID NO: 46), X₁RWWLRWAPRWFPSWRWWR (SEQ ID NO: 47), and X₁RWWLRWASRWAPSWRWWR (SEQ ID NO: 48), wherein X₁ is beta-A or S. In some embodiments, the VEPEP-9 peptide comprises the amino acid sequence of X₁WWX₄X₅WAX₆X₇X₈RX₁₀WWR (SEQ ID NO: 49), wherein X₁ is beta-A or S, X₄ is R or G, X₅ is W or S, X₆ is S, T or P, X₇ is W or P, X₈ is A or R, and X₁₀ is S or R. In some embodiments, the VEPEP-9 peptide comprises an amino acid sequence selected from the group consisting of X₁WWRWWASWARSWWR (SEQ ID NO: 50), X₁WWGSWATPRRRWWR (SEQ ID NO: 51), and X₁WWRWWAPWARSWWR (SEQ ID NO: 52), wherein X₁ is beta-A or S. In some embodiments, the VEPEP-9 peptide is present in a virus delivery complex. In some embodiments, the VEPEP-9 peptide is present in a virus delivery complex in the core of a nanoparticle. In some embodiments, the VEPEP-9 peptide is present in the core of a nanoparticle. In some embodiments, the VEPEP-9 peptide is present in the core of a nanoparticle and is associated with a virus. In some embodiments, the VEPEP-9 peptide is present in an intermediate layer of a nanoparticle. In some embodiments, the VEPEP-9 peptide is present in the surface layer of a nanoparticle. In some embodiments, the VEPEP-9 peptide is linked to a targeting moiety. In some embodiments, the linkage is covalent. In some embodiments, the covalent linkage is by chemical coupling. In some embodiments, the covalent linkage is by genetic methods.

In some embodiments, a virus delivery complex or nanoparticle described herein comprises an ADGN-100 cell-penetrating peptide comprising the amino acid sequence X₁KWRSX₂X₃X₄RWRLWRX₅X₆X₇X₈SR (SEQ ID NO: 53), wherein X₁ is any amino acid or none, and X₂-X₈ are any amino acid. In some embodiments, the ADGN-100 peptide comprises the amino acid sequence X₁KWRSX₂X₃X₄RWRLWRX₅X₆X₇X₈SR (SEQ ID NO: 54), wherein X₁ is BA, S, or none, X₂ is A or V, X₃ is or L, X₄ is W or Y, X₅ is V or S, X₆ is I, V, or A, X₇ is S or L, and X₈ is W or Y. In some embodiments, the ADGN-100 peptide comprises the amino acid sequence KWRSSAGWRWRLWRVRSWSR (SEQ ID NO: 55), KWRSALYRWRLWRVRSWSR (SEQ ID NO: 56), KWRSALYRWRLWRSRSRSWSR (SEQ ID NO: 57), or KWRSALYRWRLWRSALYSR (SEQ ID NO: 58). In some embodiments, the ADGN-100 peptide comprises two residues separated by three or six residues that are linked by a hydrocarbon linkage. In some embodiments, the ADGN-100 peptide comprises the amino acid sequence KWRS_{S}AGWR_{S}WRLWRVRSWSR (SEQ ID NO: 59), KWR_{S}SAGWRWR_{S}LWRVRSWSR (SEQ ID NO: 60). KWRSAGWR_{S}WRLWRVR_{S}SWSR (SEQ ID NO: 61), KWRS_{S}ALYR_{S}WRLWRSRSWSR (SEQ ID NO: 62), KWR_{S}SALYRWR_{S}LWRSRSWSR (SEQ ID NO: 63), KWRSALYR_{S}WRLWRSR_{S}SWSR (SEQ ID NO: 64), KWRSALYRWR_{S}LWRS_{S}RSWSR (SEQ ID NO: 65), KWRSALYRWRLWRS_{S}RSWS_{S}R (SEQ ID NO: 66), KWR_{S}SALYRWR_{S}LWRSALYSR (SEQ ID NO: 67), KWRS_{S}ALYR_{S}WRLWRSALYSR (SEQ ID NO: 68), KWRSALYRWR_{S}LWRS_{S}ALYSR (SEQ ID NO: 69), or KWRSALYRWRLWRS_{S}ALYS_{S}R (SEQ ID NO: 70), wherein the residues marked with a subscript “S” are linked by a hydrocarbon linkage. In some embodiments, the ADGN-100 peptide is present in a virus delivery complex. In some embodiments, the ADGN-100 peptide is present in a virus delivery complex in the core of a nanoparticle. In some embodiments, the ADGN-100 peptide is present in the core of a nanoparticle. In some embodiments, the ADGN-100 peptide is present in the core of a nanoparticle and is associated with a virus. In some embodiments, the ADGN-100 peptide is present in an intermediate layer of a nanoparticle. In some embodiments, the ADGN-100 peptide is present in the surface layer of a nanoparticle. In some embodiments, the ADGN-100 peptide is linked to a targeting moiety. In some embodiments, the linkage is covalent. In some embodiments, the covalent linkage is by chemical coupling. In some embodiments, the covalent linkage is by genetic methods.

In some embodiments, the CPP described herein (e.g., PEP-1, PEP-2, VEPEP-3 peptide, VEPEP-6 peptide, VEPEP-9 peptide, or ADGN-100 peptide) further comprises one or more moieties linked to the N-terminus or C-terminus of the CPP. In some embodiments, the one or more moieties is covalently linked to the N-terminus of the CPP. In some embodiments, the one or more moieties are selected from the group consisting of an acetyl group, a stearyl group, a fatty acid, a cholesterol, a poly-ethylene glycol, a nuclear localization signal, a nuclear export signal, an antibody or antibody fragment thereof, a peptide, a polysaccharide, and a targeting molecule. In some embodiments, the one or more moieties is an acetyl group and/or a stearyl group. In some embodiments, the CPP comprises an acetyl group and/or a stearyl group linked to its N-terminus. In some embodiments, the CPP comprises an acetyl group linked to its N-terminus. In some embodiments, the CPP comprises a stearyl group linked to its N-terminus. In some embodiments, the CPP comprises an acetyl group and/or a stearyl group covalently linked to its N-terminus. In some embodiments, the CPP comprises an acetyl group covalently linked to its N-terminus. In some embodiments, the CPP comprises a stearyl group covalently linked to its N-terminus.

In some embodiments, the CPP described herein (e.g., PEP-1. PEP-2, VEPEP-3 peptide, VEPEP-6 peptide, VEPEP-9 peptide, or ADGN-100 peptide) further comprises one or more moieties linked to the C-terminus of the CPP. In some embodiments, the one or more moieties is covalently linked to the C-terminus of the CPP. In some embodiments, the one or more moieties are selected from the group consisting of a cysteamide group, a cysteine, a thiol, an amide, a nitrilotriacetic acid, a carboxyl group, a linear or ramified C₁-C₆ alkyl group, a primary or secondary amine, an osidic derivative, a lipid, a phospholipid, a fatty acid, a cholesterol, a poly-ethylene glycol, a nuclear localization signal, a nuclear export signal, an antibody or antibody fragment thereof, a peptide, a polysaccharide, and a targeting molecule. In some embodiments, the one or more moieties is a cysteamide group. In some embodiments, the CPP comprises a cysteamide group linked to its C-terminus. In some embodiments, the CPP comprises a cysteamide group covalently linked to its C-terminus.

In some embodiments, the CPP described herein (e.g., PEP-1, PEP-2, VEPEP-3 peptide, VEPEP-6 peptide, VEPEP-9 peptide, or ADGN-100 peptide) further comprises one or more moieties. In some embodiments, the one or more moieties is conjugated to the N-terminus or the C-terminus of the CPP. In some embodiments, a first moiety is conjugated to the N-terminus of the CPP and a second moiety is conjugated to the C-terminus of the CPP

In some embodiments, the one or more moieties comprise a targeting molecule. In some embodiments, the targeting molecule is conjugated to the N-terminus or the C-terminus of the CPP. In some embodiments, a first targeting molecule is conjugated to the N-terminus of the CPP and a second targeting molecule is conjugated to the C-terminus of the CPP. In some embodiments, the targeting molecule comprises at least about 3, 4, or 5 amino acids. In some embodiments, the targeting molecule comprises no more than about 8, 7, 6, 5, or 4 amino acids. In some embodiments, the targeting molecule comprises about 3, 4, or 5 amino acids. In some embodiments, the targeting molecule comprises a sequence selected from the group consisting of GY, YV, VS, SK, GYV, YVS, VSK, GYVS, YVSK, YI, IG, GS, SR, YIG, IGS, GSR, YIGS, IGSR. In some embodiments, the sequence (e.g., a targeting sequence) is selected from the group consisting of GYVSK, GYVS, YIGS, and YIGSR. In some embodiments, the targeting molecule is conjugated to the CPP via a linker. In some embodiments, the linker comprises a polyglycine linker. In some embodiments, the linker comprises a β-Alanine. In some embodiments, the linker comprises at least about two, three, or four glycines, optionally continuous glycines. In some embodiments, the linker further comprises a serine. In some embodiments, the linker comprises a GGGGS or SGGGG sequence. In some embodiments, the linker comprises a Glycine-β-Alanine motif.

In some embodiments, the one or more moieties comprise a polymer (e.g., PEG, polylysine, PET). In some embodiments, the polymer is conjugated to the N-terminus or the C-terminus of the CPP. In some embodiments, a first polymer is conjugated to the N-terminus of the CPP and a second polymer is conjugated to the C-terminus of the CPP. In some embodiments, the polymer is a PEG. In some embodiments, the PEG is a linear PEG. In some embodiments, the PEG is a branched PEG. In some embodiments, the molecular weight of the PEG is no more than about 5 kDa, 10 kDa, 15 kDa, 20 kDa, 30 kDa, or 40 kDa. In some embodiments, the molecular weight of the PEG is at least about 5 kDa, 10 kDa, 15 kDa, 20 kDa, 30 kDa, or 40 kDa. In some embodiments, the molecular weight of the PEG is about 5 kDa to about 10 kDa, about 10 kDa to about 15 kDa, about 15 kDa to about 20 kDa, about 20 kDa to about 30 kDa, or about 30 kDa to about 40 kDa. In some embodiments, the molecular weight of the PEG is about 5 kDa, 10 kDa, 20 kDa. or 40 kDa. In some embodiments, the molecular weight of the PEG is selected from the group consisting of 5 kDa, 10 kDa, 20 kDa or 40 kDa. In some embodiments, the molecular weight of the PEG is about 5 kDa. In some embodiments, the molecular weight of the PEG is about 10 kDa. In some embodiments, the PEG comprises at least about 1, 2, or 3 ethylene glycol units. In some embodiments, the PEG comprises no more than about 3, 2, or 1 ethylene glycol units. In some embodiments, the PEG comprises about 1, 2, or 3 ethylene glycol units.

In some embodiments, the one or more moiety comprises a dopamine. In some embodiments, the dopamine is dopamine conjugated to the N-terminus or the C-terminus of the CPP. In some embodiments, a first dopamine is conjugated to the N-terminus of the CPP and a second dopamine is conjugated to the C-terminus of the CPP. In some embodiments, the dopamine is conjugated to the CPP via a linker. In some embodiments, the linker comprises a polyglycine linker. In some embodiments, the linker comprises a β-Alanine. In some embodiments, the linker comprises at least about two, three, or four glycines, optionally continuous glycines. In some embodiments, the linker further comprises a serine. In some embodiments, the linker comprises a GGGGS or SGGGG sequence. In some embodiments, the linker comprises a Glycine-β-Alanine motif.

In some embodiments, the CPP described herein (e.g., PEP-1, PEP-2, VEPEP-3 peptide, VEPEP-6 peptide, VEPEP-9 peptide, or ADGN-100 peptide) is stapled. “Stapled” as used herein refers to a chemical linkage between two residues in a peptide. In some embodiments, the CPP is stapled, comprising a chemical linkage between two amino acids of the peptide. In some embodiments, the two amino acids linked by the chemical linkage are separated by 3 or 6 amino acids. In some embodiments, two amino acids linked by the chemical linkage are separated by 3 amino acids. In some embodiments, the two amino acids linked by the chemical linkage are separated by 6 amino acids. In some embodiments, each of the two amino acids linked by the chemical linkage is R or S. In some embodiments, each of the two amino acids linked by the chemical linkage is R. In some embodiments, each of the two amino acids linked by the chemical linkage is S. In some embodiments, one of the two amino acids linked by the chemical linkage is R and the other is S. In some embodiments, the chemical linkage is a hydrocarbon linkage.

In some embodiments, the CPP is an L-peptide comprising L-amino acids. In some embodiments, the CPP is a retro-inverso peptide (e.g., a peptide made up of D-amino acids in a reversed sequence and, when extended, assumes a side chain topology similar to that of its parent molecule but with inverted amide peptide bonds).

Also provided are all the CPPs described herein.

### Complexes

In some embodiments, there is provided a virus delivery complex for intracellular delivery of a virus comprising a cell-penetrating peptide (e.g., a PEP-1, PEP-2, VEPEP-3, VEPEP-6, VEPEP-9, or ADGN-100 peptide) associated with one or more viruses. In some embodiments, the association is non-covalent. In some embodiments, the association is covalent. In some embodiments, the virus is a recombinant virus, including recombinant AAV, adenovirus, lentivirus, retrovirus, HSV, poxvirus, EBV, vaccinia virus, and hCMV. In some embodiments, the recombinant virus comprises a transgene for insertion into a cell genome. In some embodiments, the transgene is a therapeutic transgene. In some embodiments, at least some of the cell-penetrating peptides in the virus delivery complex are linked to a targeting moiety. In some embodiments, the linkage is covalent. In some embodiments, the covalent linkage is by chemical coupling. In some embodiments, the covalent linkage is by genetic methods. In some embodiments, the molar ratio of cell-penetrating peptide to at least one of the one or more viruses (e.g., in Vg, pfu, or MOI) is between about 1:1 and about 1×10⁸:1. In some embodiments, the CPP includes, but is not limited to, a PTD-based peptide, an amphipathic peptide, a poly-arginine-based peptide, an MPG peptide, a CADY peptide, a VEPEP peptide (such as a VEPEP-3, VEPEP-6, or VEPEP-9 peptide), an ADGN-100 peptide, a Pep-1 peptide, and a Pep-2 peptide. In some embodiments, the virus delivery complex comprises a virus for introducing a specific modification to a target polynucleotide. In some embodiments, the target polynucleotide is modified in a coding sequence. In some embodiments, the target polynucleotide is modified in a non-coding sequence. In some embodiments, the target polynucleotide is modified to inactivate a target gene, such as by decreasing expression of the target gene or resulting in a modified target gene that expresses an inactive product. In some embodiments, the target polynucleotide is modified to activate a target gene, such as by increasing expression of the target gene or resulting in a modified target gene that expresses an active target gene product. In some embodiments, the transgene encodes a protein, such as a therapeutic protein. In some embodiments, the transgene encodes an inhibitory RNA (RNAi), such as an RNAi targeting an endogenous gene, e.g., a disease-associated endogenous gene. In some embodiments, the transgene encodes a CAR In some embodiments, the complex comprises one or more viruses comprising a first transgene encoding an RNAi and a second transgene encoding a protein. In some embodiments, the RNAi is a therapeutic RNAi targeting an endogenous gene involved in a disease or condition, and the protein is a therapeutic protein useful for treating the disease or condition. In some embodiments, the therapeutic RNAi targets a disease-associated form of the endogenous gene (e.g., a gene encoding a mutant protein, or a gene resulting in abnormal expression of a protein), and the second transgene is a therapeutic form of the endogenous gene (e.g., the second transgene encodes a wild-type or functional form of the mutant protein, or the second transgene results in normal expression of the protein). In some embodiments, the complex comprises a first virus comprising the first transgene and a second virus comprising the second transgene. In some embodiments, the complex comprises a single virus comprising the first transgene and the second transgene.

CPPs can be covalently associated to virus (such as AAV) using either chemical conjugation or genetic recombination. For example, CPPs can be linked to virus via cross linking involving either C-terminal cysteamide/cysteine or an N-terminal beta-Alanine bridge. Virus can also be covalently linked to various moieties inside a peptide chain using any technique known in the art for such purposes, including for example chemistry such as 6-maleimidohexanoic acid N-hydroxysuccinimide ester. See for example Kurachi, S., et al. Gene therapy 14.15 (2007): 1160; and Yu, Di, et al. Journal of virology 85.24 (2011): 13114-13123.

In some embodiments, there is provided a virus delivery complex for introducing a modification to a target polynucleotide comprising a cell-penetrating peptide (e.g., a PEP-1. PEP-2. VEPEP-3, VEPEP-6, VEPEP-9, or ADGN-100 peptide) and a virus that targets the target polynucleotide. In some embodiments, the modification is addition, deletion, or substitution of one or more nucleotides in the target polynucleotide. In some embodiments, the modification is insertion of a heterologous nucleic acid in the target polynucleotide. In some embodiments, at least some of the cell-penetrating peptides in the virus delivery complex are linked to a targeting moiety. In some embodiments, the linkage is covalent. In some embodiments, the covalent linkage is by chemical coupling. In some embodiments, the covalent linkage is by genetic methods. In some embodiments, the molar ratio of cell-penetrating peptide to at least one of the one or more viruses (e.g., in Vg, pfu, or MOI) is between about 1:1 and about 1×10⁸:1. In some embodiments, the CPP includes, but is not limited to, a PTD-based peptide, an amphipathic peptide, a poly-arginine-based peptide, an MPG peptide, a CADY peptide, a VEPEP peptide (such as a VEPEP-3, VEPEP-6, or VEPEP-9 peptide), an ADGN-100 peptide, a Pep-1 peptide, and a Pep-2 peptide. In some embodiments, the target polynucleotide is modified in a coding sequence. In some embodiments, the target polynucleotide is modified in a non-coding sequence. In some embodiments, the target polynucleotide is modified to inactivate a target gene, such as by decreasing expression of the target gene or resulting in a modified target gene that expresses an inactive product. In some embodiments, the target polynucleotide is modified to activate a target gene, such as by increasing expression of the target gene or resulting in a modified target gene that expresses an active target gene product.

In some embodiments, there is provided a virus delivery complex for modifying one or more target polynucleotides comprising a cell-penetrating peptide (e.g., a PEP-1, PEP-2, VEPEP-3. VEPEP-6. VEPEP-9, or ADGN-100 peptide) and a plurality of viruses, wherein each of the plurality of viruses targets a different sequence in the one or more target polynucleotides. In some embodiments, at least some of the cell-penetrating peptides in the virus delivery complex are linked to a targeting moiety. In some embodiments, the linkage is covalent. In some embodiments, the covalent linkage is by chemical coupling. In some embodiments, the covalent linkage is by genetic methods. In some embodiments, the molar ratio of cell-penetrating peptide to at least one of the one or more viruses (e.g., in Vg, pfu, or MOI) is between about 1:1 and about 1×10⁸:1. In some embodiments, the CPP includes, but is not limited to, a PTD-based peptide, an amphipathic peptide, a poly-arginine-based peptide, an MPG peptide, a CADY peptide, a VEPEP peptide (such as a VEPEP-3, VEPEP-6, or VEPEP-9 peptide), an ADGN-100 peptide, a Pep-1 peptide, and a Pep-2 peptide. In some embodiments, one of the one or more target polynucleotides is modified in a coding sequence. In some embodiments, one of the one or more target polynucleotides is modified in a non-coding sequence. In some embodiments, one of the one or more target polynucleotides is modified to inactivate a target gene, such as by decreasing expression of the target gene or resulting in a modified target gene that expresses an inactive product. In some embodiments, one of the one or more target polynucleotides is modified to activate a target gene, such as by increasing expression of the target gene or resulting in a modified target gene that expresses an active target gene product.

In some embodiments, there is provided a virus delivery complex for intracellular delivery of a virus comprising a cell-penetrating peptide associated with the virus, wherein the cell-penetrating peptide comprises the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80.

In some embodiments, there is provided a virus delivery complex for introducing a modification to a target polynucleotide comprising a cell-penetrating peptide and a virus, wherein the virus targets the target polynucleotide, and wherein the cell-penetrating peptide comprises the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the modification is addition, deletion, or substitution of one or more nucleotides in the target polynucleotide. In some embodiments, the modification is insertion of a heterologous nucleic acid in the target polynucleotide.

In some embodiments, there is provided a virus delivery complex for modifying one or more target polynucleotides comprising a cell-penetrating peptide and a plurality of viruses, wherein each of the plurality of viruses targets a different sequence in the one or more target polynucleotides, and wherein the cell-penetrating peptide comprises the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80.

In some embodiments, a virus contained in a virus delivery complex according to any of the embodiments described herein targets a gene encoding a protein involved in regulating an immune response, including immune checkpoint regulators and proteins involved in antigen presentation. In some embodiments, a virus contained in a virus delivery complex according to any of the embodiments described herein targets a gene encoding a protein involved in regulating cholesterol transport and/or metabolism. In some embodiments, the virus targets a sequence in a gene encoding a protein including, without limitation, PD-1, PD-L1, PD-L2, TIM-1, TIM-3, TIM-4, BTLA, VISTA, LAG-3, CTLA-4, TIGIT, 4-1BB, OX40, CD27, CD28, HVEM, GITR, ICOS, CD40, CD80, CD86, B7-H2, B7-H3, B7-H4, B7-H6, 2B4, CD160, gp49B, PIR-B, KIR family receptors, SIRPalpha (CD47), CD48, 2B4 (CD244), B7.1, B7.2, ILT-2, ILT-4, A2aR, toll-like receptors TLR-2, 3, 4, 6, 7, 8, and 9, granulocyte macrophage colony stimulating factor (GM-CSF), TNF, CD40L, FLT-3 ligand, cytokines such as IL-1, IL-2, IL-4, IL-7, IL-10, IL-12, IL-15, IL-21, and IL-35, FasL, TGF-β, indoleamine-2.3 dioxygenase (IDO), major histocompatibility complex (MHC) proteins, including beta-2 microglobulin (β2M), low-density lipoprotein (LDL) receptor (LDLR), apolipoprotein B (ApoB), low-density lipoprotein receptor adapter protein 1 (LDLRAP1), and proprotein convertase subtilisin kexin 9 (PCSK9).

In some embodiments, according to any of the virus delivery complexes described herein, the virus delivery complex further comprises a protein other than a viral protein, or a nucleic acid molecule other than a viral nucleic acid molecule (such as a nucleic acid molecule encoding a non-viral protein, e.g., a DNA plasmid or mRNA).

In some embodiments, the mean size (diameter) of a virus delivery complex described herein is between any of about 20 nm and about 10 microns, including for example between about 30 nm and about 1 micron, between about 50 nm and about 750 nm, between about 100 nm and about 500 nm, and between about 200 nm and about 400 nm. In some embodiments, the virus delivery complex is substantially non-toxic.

In some embodiments, the targeting moiety of a virus delivery complex described herein targets the virus delivery complex to a tissue or a specific cell type. In some embodiments, the tissue is a tissue in need of treatment. In some embodiments, the targeting moiety targets the virus delivery complex to a tissue or cell that can be treated by the virus.

### Nanoparticles

In some embodiments, there is provided a nanoparticle for intracellular delivery of a virus comprising a core comprising one or more virus delivery complexes described herein. In some embodiments, the nanoparticle core comprises a plurality of virus delivery complexes. In some embodiments, the nanoparticle core comprises a plurality of virus delivery complexes present in a predetermined ratio. In some embodiments, the predetermined ratio is selected to allow the most effective use of the nanoparticle in any of the methods described below in more detail. In some embodiments, the nanoparticle core further comprises one or more additional cell-penetrating peptides and/or one or more additional viruses. In some embodiments, the nanoparticle core further comprises one or more additional cell-penetrating peptides associated with (such as covalently or non-covalently) one or more additional viruses. In some embodiments, the one or more additional cell-penetrating peptides does not comprise a cell-penetrating peptide found in any of the one or more virus delivery complexes. In some embodiments, the one or more additional viruses does not comprise a virus found in any of the one or more virus delivery complexes. In some embodiments, the one or more additional cell-penetrating peptides include, but are not limited to, a PTD-based peptide, an amphipathic peptide, a poly-arginine-based peptide, an MPG peptide, a CADY peptide, a VEPEP peptide (such as a VEPEP-3, VEPEP-6, or VEPEP-9 peptide), an ADGN-100 peptide, a Pep-1 peptide, and a Pep-2 peptide. In some embodiments, at least some of the one or more additional cell-penetrating peptides are linked to a targeting moiety. In some embodiments, the linkage is covalent. In some embodiments, the covalent linkage is by chemical coupling. In some embodiments, the covalent linkage is by genetic methods.

In some embodiments, there is provided a nanoparticle for intracellular delivery of a virus comprising a core comprising one or more cell-penetrating peptides (e.g., a PEP-1, PEP-2, VEPEP-3, VEPEP-6, VEPEP-9, or ADGN-100 peptide) associated with the virus. In some embodiments, the association is non-covalent. In some embodiments, the association is covalent. In some embodiments, the virus is a recombinant virus, including recombinant AAV, adenovirus, lentivirus, retrovirus, HSV, poxvirus, EBV, vaccinia virus, and hCMV. In some embodiments, the recombinant virus comprises a transgene for insertion into a cell genome. In some embodiments, the transgene is a therapeutic transgene. In some embodiments, at least some of the one or more cell-penetrating peptides in the nanoparticle are linked to a targeting moiety. In some embodiments, the linkage is covalent. In some embodiments, the covalent linkage is by chemical coupling. In some embodiments, the covalent linkage is by genetic methods. In some embodiments, the molar ratio of a cell-penetrating peptide to a virus (e.g., in Vg, pfu, or MOI) associated with the cell-penetrating peptide in a complex present in the nanoparticle is between about 1:1 and about 1×10⁸:1. In some embodiments, the virus is for introducing a specific modification to a target polynucleotide. In some embodiments, the target polynucleotide is modified in a coding sequence. In some embodiments, the target polynucleotide is modified in a non-coding sequence. In some embodiments, the target polynucleotide is modified to inactivate a target gene, such as by decreasing expression of the target gene or resulting in a modified target gene that expresses an inactive product. In some embodiments, the target polynucleotide is modified to activate a target gene, such as by increasing expression of the target gene or resulting in a modified target gene that expresses an active target gene product. In some embodiments, the transgene encodes a protein, such as a therapeutic protein. In some embodiments, the transgene encodes an inhibitory RNA (RNAi), such as an RNAi targeting an endogenous gene. e.g., a disease-associated endogenous gene. In some embodiments, the transgene encodes a CAR. In some embodiments, the nanoparticle comprises one or more viruses comprising a first transgene encoding an RNAi and a second transgene encoding a protein. In some embodiments, the RNAi is a therapeutic RNAi targeting an endogenous gene involved in a disease or condition, and the protein is a therapeutic protein useful for treating the disease or condition. In some embodiments, the therapeutic RNAi targets a disease-associated form of the endogenous gene (e.g., a gene encoding a mutant protein, or a gene resulting in abnormal expression of a protein), and the second transgene is a therapeutic form of the endogenous gene (e.g., the second transgene encodes a wild-type or functional form of the mutant protein, or the second transgene results in normal expression of the protein). In some embodiments, the nanoparticle comprises a first virus comprising the first transgene and a second virus comprising the second transgene. In some embodiments, the nanoparticle comprises a single virus comprising the first transgene and the second transgene. In some embodiments, the one or more cell-penetrating peptides include, but are not limited to, a PTD-based peptide, an amphipathic peptide, a poly-arginine-based peptide, an MPG peptide, a CADY peptide, a VEPEP peptide (such as a VEPEP-3, VEPEP-6, or VEPEP-9 peptide), an ADGN-100 peptide, a Pep-1 peptide, and a Pep-2 peptide.

In some embodiments, there is provided a nanoparticle for modifying one or more target polynucleotides comprising a core comprising one or more cell-penetrating peptides (e.g., a PEP-1, PEP-2, VEPEP-3, VEPEP-6, VEPEP-9, or ADGN-100 peptide) and a plurality of viruses, wherein each of the plurality of viruses targets a different sequence in the one or more target polynucleotides. In some embodiments, the nanoparticle core comprises one of the one or more cell-penetrating peptides associated with at least one of the plurality of viruses. In some embodiments, the nanoparticle core comprises a) a first complex comprising one of the one or more cell-penetrating peptides associated with at least one of the plurality of viruses, and b) one or more additional complexes comprising the remaining cell-penetrating peptides associated with the remaining viruses. In some embodiments, at least some of the one or more cell-penetrating peptides in the nanoparticle are linked to a targeting moiety. In some embodiments, the linkage is covalent. In some embodiments, the covalent linkage is by chemical coupling. In some embodiments, the covalent linkage is by genetic methods. In some embodiments, the molar ratio of a cell-penetrating peptide to a virus (e.g., in Vg, pfu, or MOI) associated with the cell-penetrating peptide in a complex present in the nanoparticle is between about 1:1 and about 1×10⁸:1. In some embodiments, one of the one or more target polynucleotides is modified in a coding sequence. In some embodiments, one of the one or more target polynucleotides is modified in a non-coding sequence. In some embodiments, one of the one or more target polynucleotides is modified to inactivate a target gene, such as by decreasing expression of the target gene or resulting in a modified target gene that expresses an inactive product. In some embodiments, one of the one or more target polynucleotides is modified to activate a target gene, such as by increasing expression of the target gene or resulting in a modified target gene that expresses an active target gene product. In some embodiments, the one or more cell-penetrating peptides include, but are not limited to, a PTD-based peptide, an amphipathic peptide, a poly-arginine-based peptide, an MPG peptide, a CADY peptide, a VEPEP peptide (such as a VEPEP-3, VEPEP-6, or VEPEP-9 peptide), an ADGN-100 peptide, a Pep-1 peptide, and a Pep-2 peptide.

In some embodiments, there is provided a nanoparticle for intracellular delivery of a virus comprising a core comprising a cell-penetrating peptide and a virus, wherein the cell-penetrating peptide is associated with the virus, and wherein the cell-penetrating peptide comprises the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80.

In some embodiments, there is provided a nanoparticle for modifying one or more target polynucleotides comprising a core comprising a cell-penetrating peptide and a plurality of viruses, wherein each of the plurality of viruses targets a different sequence in the one or more target polynucleotides, and wherein the cell-penetrating peptide comprises the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the nanoparticle core comprises the cell-penetrating peptide associated with at least one of the plurality of viruses. In some embodiments, the nanoparticle core comprises a) a first complex comprising the cell-penetrating peptide associated with at least one of the plurality of viruses, and b) one or more additional complexes comprising the cell-penetrating peptide associated with the remaining viruses.

In some embodiments, the nanoparticle further comprises a surface layer comprising a peripheral CPP surrounding the core. In some embodiments, the peripheral CPP is the same as a CPP in the core. In some embodiments, the peripheral CPP is different than any of the CPPs in the core. In some embodiments, the peripheral CPP includes, but is not limited to, a PTD-based peptide, an amphipathic peptide, a poly-arginine-based peptide, an MPG peptide, a CADY peptide, a VEPEP peptide (such as a VEPEP-3, VEPEP-6, or VEPEP-9 peptide), an ADGN-100 peptide, a Pep-1 peptide, and a Pep-2 peptide. In some embodiments, the peripheral CPP is a VEPEP-3 peptide, a VEPEP-6 peptide, a VEPEP-9 peptide, or an ADGN-100 peptide. In some embodiments, at least some of the peripheral cell-penetrating peptides in the surface layer are linked to a targeting moiety. In some embodiments, the linkage is covalent. In some embodiments, the covalent linkage is by chemical coupling. In some embodiments, the covalent linkage is by genetic methods. In some embodiments, the nanoparticle further comprises an intermediate layer between the core of the nanoparticle and the surface layer. In some embodiments, the intermediate layer comprises an intermediate CPP. In some embodiments, the intermediate CPP is the same as a CPP in the core. In some embodiments, the intermediate CPP is different than any of the CPPs in the core. In some embodiments, the intermediate CPP includes, but is not limited to, a PTD-based peptide, an amphipathic peptide, a poly-arginine-based peptide, an MPG peptide, a CADY peptide, a VEPEP peptide (such as a VEPEP-3, VEPEP-6, or VEPEP-9 peptide), an ADGN-100 peptide, a Pep-1 peptide, and a Pep-2 peptide. In some embodiments, the intermediate CPP is a VEPEP-3 peptide, a VEPEP-6 peptide, a VEPEP-9 peptide, or an ADGN-100 peptide.

In some embodiments, a virus contained in a nanoparticle according to any of the embodiments described herein targets a sequence in a gene encoding a protein involved in regulating an immune response, including immune checkpoint regulators and proteins involved in antigen presentation. In some embodiments, a virus contained in a nanoparticle according to any of the embodiments described herein targets a sequence in a gene encoding a protein involved in regulating cholesterol transport and/or metabolism. In some embodiments, the virus targets a sequence in a gene encoding a protein including, without limitation, PD-1, PD-L1, PD-L2, TIM-1, TIM-3, TIM-4, BTLA. VISTA, LAG-3, CTLA-4, TIGIT, 4-1BB, OX40, CD27, CD28, HVEM, GITR, ICOS, CD40, CD80, CD86, B7-H2, B7-H3, B7-H4, B7-H6, 2B4, CD160, gp49B, PIR-B, KIR family receptors, SIRPalpha (CD47), CD48, 2B4 (CD244), B7.1, B7.2, ILT-2, ILT-4, A2aR, toll-like receptors TLR-2, 3, 4, 6, 7, 8, and 9, granulocyte macrophage colony stimulating factor (GM-CSF), TNF, CD40L, FLT-3 ligand, cytokines such as IL-1, IL-2, IL-4, IL-7, IL-10, IL-12, IL-15, IL-21, and IL-35, FasL, TGF-β, indoleamine-2,3 dioxygenase (IDO), major histocompatibility complex (MHC) proteins, including beta-2 microglobulin (β2M), LDLR, ApoB, LDLRAP1, and PCSK9.

In some embodiments, according to any of the nanoparticles described herein, the nanoparticle further comprises a protein other than a viral protein, or a nucleic acid molecule other than a viral nucleic acid molecule (such as a nucleic acid molecule encoding a non-viral protein, e.g., a DNA plasmid or mRNA).

In some embodiments, according to any of the nanoparticles described herein, the mean size (diameter) of the nanoparticle is from about 20 nm to about 1000 nm, including for example from about 50 nm to about 800 nm, from about 75 nm to about 600 nm, from about 100 nm to about 600 nm, and from about 200 nm to about 400 nm. In some embodiments, the mean size (diameter) of the nanoparticle is no greater than about 1000 nanometers (nm), such as no greater than about any of 900, 800, 700, 600, 500, 400, 300, 200, or 100 nm. In some embodiments, the average or mean diameter of the nanoparticle is no greater than about 200 nm. In some embodiments, the average or mean diameters of the nanoparticles is no greater than about 150 nm. In some embodiments, the average or mean diameter of the nanoparticle is no greater than about 100 nm. In some embodiments, the average or mean diameter of the nanoparticle is about 20 nm to about 400 nm. In some embodiments, the average or mean diameter of the nanoparticle is about 30 nm to about 400 nm. In some embodiments, the average or mean diameter of the nanoparticle is about 40 nm to about 300 nm. In some embodiments, the average or mean diameter of the nanoparticle is about 50 nm to about 200 nm. In some embodiments, the average or mean diameter of the nanoparticle is about 60 nm to about 150 nm. In some embodiments, the average or mean diameter of the nanoparticle is about 70 nm to about 100 nm. In some embodiments, the nanoparticles are sterile-filterable.

In some embodiments, the zeta potential of the nanoparticle is from about −30 mV to about 60 mV (such as about any of −30, −25, −20, −15, −10, −5, 0, 5, 10, 15, 20, 25, 30, 35, 40, 45, 50, 55, and 60 mV, including any ranges between these values). In some embodiments, the zeta potential of the nanoparticle is from about −30 mV to about 30 mV, including for example from about −25 mV to about 25 mV, from about −20 mV to about 20 mV, from about −15 mV to about 15 mV, from about −10 mV to about 10 mV, and from about −5 mV to about 10 mV. In some embodiments, the polydispersity index (PI) of the nanoparticle is from about 0.05 to about 0.6 (such as about any of 0.05, 0.1, 0.15, 0.2, 0.25, 0.3, 0.35, 0.4, 0.45, 0.5, 0.55, and 0.6, including any ranges between these values). In some embodiments, the nanoparticle is substantially non-toxic.

### Modifications

In some embodiments, a virus delivery complex or nanoparticle as described herein comprises a targeting moiety, wherein the targeting moiety is a ligand capable of cell-specific and/or nuclear targeting. A cell membrane surface receptor and/or cell surface marker is a molecule or structure which can bind said ligand with high affinity and preferably with high specificity. Said cell membrane surface receptor and/or cell surface marker is preferably specific for a particular cell, i.e. it is found predominantly in one type of cell rather than in another type of cell (e.g. galactosyl residues to target the asialoglycoprotein receptor on the surface of hepatocytes). The cell membrane surface receptor facilitates cell targeting and internalization into the target cell of the ligand (e.g. the targeting moiety) and attached molecules (e.g. the complex or nanoparticle of the invention). A large number of ligand moieties/ligand binding partners that may be used in the context of the present invention are widely described in the literature. Such a ligand moiety is capable of conferring to the complex or nanoparticle of the invention the ability to bind to a given binding-partner molecule or a class of binding-partner molecules localized at the surface of at least one target cell. Suitable binding-partner molecules include without limitation polypeptides selected from the group consisting of cell-specific markers, tissue-specific markers, cellular receptors, viral antigens, antigenic epitopes and tumor-associated markers. Binding-partner molecules may moreover consist of or comprise, for example, one or more sugar, lipid, glycolipid, antibody molecules or fragments thereof, or aptamer. According to the invention, a ligand moiety may be for example a lipid, a glycolipid, a hormone, a sugar, a polymer (e.g. PEG, polylysine, PET), an oligonucleotide, a vitamin, an antigen, all or part of a lectin, all or part of a polypeptide, such as for example JTS1 (WO 94/40958), an antibody or a fragment thereof, or a combination thereof. In some embodiments, the ligand moiety used in the present invention is a peptide or polypeptide having a minimal length of 7 amino acids. It is either a native polypeptide or a polypeptide derived from a native polypeptide. “Derived” means containing (a) one or more modifications with respect to the native sequence (e.g. addition, deletion and/or substitution of one or more residues), (b) amino acid analogs, including non-naturally occurring amino acids, (c) substituted linkages, or (d) other modifications known in the art. The polypeptides serving as ligand moiety encompass variant and chimeric polypeptides obtained by fusing sequences of various origins, such as for example a humanized antibody which combines the variable region of a mouse antibody and the constant region of a human immunoglobulin. In addition, such polypeptides may have a linear or cyclized structure (e.g. by flanking at both extremities a polypeptide ligand by cysteine residues). Additionally, the polypeptide in use as a ligand moiety may include modifications of its original structure by way of substitution or addition of chemical moieties (e.g. glycosylation, alkylation, acetylation, amidation, phosphorylation, addition of sulfhydryl groups and the like). The invention further contemplates modifications that render the ligand moiety detectable. For this purpose, modifications with a detectable moiety can be envisaged (i.e. a scintigraphic, radioactive, or fluorescent moiety, or a dye label and the like). Such detectable labels may be attached to the ligand moiety by any conventional techniques and may be used for diagnostic purposes (e.g. imaging of tumoral cells). In some embodiments, the binding-partner molecule is an antigen (e.g. a target cell-specific antigen, a disease-specific antigen, an antigen specifically expressed on the surface of engineered target cells) and the ligand moiety is an antibody, a fragment or a minimal recognition unit thereof (e.g. a fragment still presenting an antigenic specificity) such as those described in detail in immunology manuals (see for example Immunology, third edition 1993, Roitt, Brostoff and Male, ed Gambli, Mosby). The ligand moiety may be a monoclonal antibody. Many monoclonal antibodies that bind many of these antigens are already known, and using techniques known in the art in relation to monoclonal antibody technology, antibodies to most antigens may be prepared. The ligand moiety may be a part of an antibody (for example a Fab fragment) or a synthetic antibody fragment (for example, ScFv). In some embodiments, the ligand moiety is selected among antibody fragments, rather than whole antibodies. Effective functions of whole antibodies, such as complement binding, are removed. ScFv and dAb antibody fragments may be expressed as a fusion with one or more other polypeptides. Minimal recognition units may be derived from the sequence of one or more of the complementary-determining regions (CDR) of the Fv fragment. Whole antibodies, and F(ab′)2 fragments are “bivalent”. By “bivalent” it is meant that said antibodies and F(ab′)2 fragments have two antigen binding sites. In contrast, Fab, Fv, ScFv, dAb fragments and minimal recognition units are monovalent, having only one antigen binding sites. In some embodiments, the ligand moiety allows targeting to a tumor cell and is capable of recognizing and binding to a molecule related to the tumor status, such as a tumor-specific antigen, a cellular protein differentially or over-expressed in tumor cells or a gene product of a cancer-associated vims. Examples of tumor-specific antigens include but are not limited to MUC-1 related to breast cancer (Harcuven i et al., 990, Eur. J. Biochem 189, 475-486), the products encoded by the mutated BRCA1 and BRCA2 genes related to breast and ovarian cancers (Miki et al, 1994, Science 226, 66-71; Fuireal et al, 1994. Science 226, 120-122; Wooster et al., 1995, Nature 378, 789-792), APC related to colon cancer (Poiakis, 1995, Curr. Opin. Genet. Dev. 5, 66-71), prostate specific antigen (PSA) related to prostate cancer, (Stamey et al., 1987, New England J. Med. 317, 909), carcinoma embryonic antigen (CEA) related to colon cancers (Schrewe et al., 1990, Mol. Cell. Biol. 10, 2738-2748), tyrosinase related to melanomas (Vile et al, 1993. Cancer Res. 53, 3860-3864), receptor for melanocyte-stimulating hormone (MSH) which is highly expressed in melanoma cells, ErbB-2 related to breast and pancreas cancers (Harris et al., 1994, Gene Therapy 1, 170-175), and alpha-foetoprotein related to liver cancers (Kanai et al., 1997, Cancer Res. 57, 461-465). In some embodiments, the ligand moiety is a fragment of an antibody capable of recognizing and binding to the MUC-1 antigen and thus targeting MUC-1 positive tumor cells. In some embodiments, the ligand moiety is the scFv fragment of the SM3 monoclonal antibody which recognizes the tandem repeat region of the MUC-1 antigen (Burshell et al., 1987, Cancer Res. 47, 5476-5482; Girling et al., 1989, Int. J. Cancer 43, 1072-1076; Dokumo et al., 1998, J. Mol. Biol. 284, 713-728). Examples of cellular proteins differentially or overexpressed in tumor cells include but are not limited to the receptor for interleukin 2 (IL-2) overexpressed in some lymphoid tumors, GRP (Gastrin Release Peptide) overexpressed in lung carcinoma cells, pancreas, prostate and stomach tumors (Michael et al., 1995, Gene Therapy 2, 660-668), TNF (Tumor Necrosis Factor) receptor, epidermal growth factor receptors. Fas receptor, CD40 receptor, CD30 receptor, CD27 receptor, OX-40, α-v integrins (Brooks et al, 994, Science 264, 569) and receptors for certain angiogenic growth factors (Hanahan, 1997, Science 277, 48). Based on these indications, it is within the scope of those skilled in the art to define an appropriate ligand moiety capable of recognizing and binding to such proteins. To illustrate, IL-2 is a suitable ligand moiety to bind to TL-2 receptor. In the case of receptors that are specific to fibrosis and inflammation, these include the TGFbeta receptors or the Adenosine receptors that are identified above and are suitable targets for invention compositions. Cell surface markers for multiple myeloma include, but are not limited to, CD56, CD40, FGFR3, CS1, CD138, IGF1R. VEGFR, and CD38, and are suitable targets for invention compositions. Suitable ligand moieties that bind to these cell surface markers include, but are not limited to, anti-CD56, anti-CD40, PRO-001, Chir-258, HuLuc63, anti-CD138-DM1, anti-IGF1R and bevacizumab.

### Transgenes

In some embodiments, a virus delivery complex or nanoparticle described herein comprises one or more viruses comprising one or more transgenes (such as about any of 1, 2, 3, 4, 5, or more transgenes). In some embodiments, one or more of the transgenes encode a protein, such as a therapeutic protein. In some embodiments, one or more of the transgenes encode an inhibitory RNA (RNAi), such as a therapeutic RNAi. In some embodiments, one or more of the transgenes encode a chimeric antigen receptor (CAR).

### Chimeric Antigen Receptors (CARs)

Exemplary antigen receptors, including CARs, and methods for engineering such receptors, include those described, for example, in international patent application publication numbers WO200014257, WO2013126726. WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398: Davila et al. (2013) PLoS ONE 8(4): e61338; Turtle et al., Curr. Opin. Immunol., 2012 October; 24(5): 633-39; Wu et al., Cancer. 2012 March 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013. Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

In some embodiments of a recombinant receptor comprising a ligand-binding domain described herein, the ligand-binding domain specifically binds to a ligand associated with a disease or condition. In some embodiments, the ligand-binding domain specifically binds to a cancer associated antigen or a pathogen-specific antigen. In some embodiments, the ligand-binding domain is specific for viral antigens (such as HIV, HCV, HBV, etc.), bacterial antigens, and/or parasitic antigens. In some embodiments, the ligand-binding domain specifically binds to a ligand including, without limitation, a orphan tyrosine kinase receptor ROR1, tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, 0EPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-A 1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, and MAGE A3, CE7, Wilms Tumor 1 (WT-1), a cyclin, such as cyclin A1 (CCNA1), and/or biotinylated molecules, and/or molecules expressed by HIV, HCV, HBV or other pathogens.

### Virus

In some embodiments, according to any of the complexes and/or nanoparticles described herein, the virus is a recombinant virus, including recombinant adeno-associated virus (AAV), adenovirus, lentivirus, retrovirus, herpes simplex virus (HSV), poxvirus. Epstein-Barr virus (EBV), vaccinia virus, and human cytomegalovirus (hCMV). In some embodiments, the recombinant virus comprises a transgene for insertion into a cell genome. In some embodiments, the transgene is a therapeutic transgene. In some embodiments, the transgene encodes a protein, such as a therapeutic protein. In some embodiments, the transgene encodes an inhibitory RNA (RNAi), such as an RNAi targeting an endogenous gene, e.g., a disease-associated endogenous gene. In some embodiments, the transgene encodes a CAR. In some embodiments, the virus comprises a first transgene encoding an RNAi. In some embodiments, the RNAi is a therapeutic RNAi targeting an endogenous gene involved in a disease or condition. In some embodiments, the therapeutic RNAi targets a disease-associated form of the endogenous gene (e.g., a gene encoding a mutant protein, or a gene resulting in abnormal expression of a protein). In some embodiments, the virus comprises a second transgene encoding a protein. In some embodiments, the protein is a therapeutic protein useful for treating a disease or condition. In some embodiments, the second transgene is a therapeutic form of an endogenous gene (e.g., the second transgene encodes a wild-type or functional form of a mutant protein encoded by the endogenous gene, or the second transgene results in normal expression of a protein encoded by the endogenous gene). In some embodiments, there is provided a virus comprising the first transgene and the second transgene.

In some embodiments, according to any of the complexes and/or nanoparticles described herein, the virus is a modified virus, including modified adeno-associated virus (AAV), adenovirus, lentivirus, retrovirus, herpes simplex virus (HSV), poxvirus, Epstein-Barr virus (EBV), vaccinia virus, and human cytomegalovirus (hCMV). In some embodiments, according to any of the complexes and/or nanoparticles described herein, the virus is an inactivated virus, including inactivated adeno-associated virus (AAV), adenovirus, lentivirus, retrovirus, herpes simplex virus (HSV), poxvirus, Epstein-Barr virus (EBV), vaccinia virus, and human cytomegalovirus (hCMV). In some embodiments, according to any of the complexes and/or nanoparticles described herein, the virus is a replication-deficient virus, including replication-deficient adeno-associated virus (AAV), adenovirus, lentivirus, retrovirus, herpes simplex virus (HSV), poxvirus, Epstein-Barr virus (EBV), vaccinia virus, and human cytomegalovirus (hCMV). In some embodiments, according to any of the complexes and/or nanoparticles described herein, the virus is only able to replicate in target cells.

### AAV

The AAV capsid is composed of 60 capsid protein subunits, VP1, VP2, and VP3, which are arranged in an icosahedral symmetry in a ratio of 1:1:10, with an estimated size of 3.9 MegaDaltons. The VP protein consists of a β-barrel structural organization at the inner surface of the capsid flanked by several loops. The surface of the VP protein contains several positively charged patches because of the predominance of ionic interactions with the sugar sulfates. Overall, the outside surface is positively charged with a prominent ring of symmetry-related patches in a depression surrounding the five-fold axes.

Therefore, the tight interaction between AAV and CPP is directly related to the ability of amphipathic CPPs to expose charge surfaces together with the presence of aromatic residues such as Trp, which are major residues in protein/protein interface. CPPs can form stable electrostatics and hydrophobic interactions with VP capsid proteins. Tryptophan residues may interact with surface exposed loops. So far 12 different serotypes of AAV have been isolated. According to the degree of similarity that a residue has with the consensus residue for each serotype a phylogenic relationship has been established for the different serotypes. The tree shows that serotype AAV5 has the most divergent amino acid capsid sequence, sharing between 53% and 59% homology with the rest of the human serotypes that have been discovered so far. AAV4 also shows a considerable degree of divergence, when comparing sequences of AAV1 to 9 (between 53% and 64%).

However, divergence occurs mainly in amino acidic sequences were mainly localized in a couple of looped-exposed at the surface of the virus instead of being distributed along the capsid sequences, suggestion that divergence may have a minor impact on the CPP/AAV interactions as CPP will cover entirely AAV.

In some embodiments, according to any of the virus delivery complexes or nanoparticles described herein, the complex or nanoparticles comprises an AAV. In some embodiments, the AAV is AAV 1, AAV2, AAV3, AAV4, AAV5, AAV6, AAV7, AAV8, AAV9, AAV10, AAV11, or AAV12. In some embodiments, the AAV is pseudotyped, comprising a capsid and genome derived from different viral serotypes. For example, in some embodiments, the AAV is any one of AAV 1/2, AAV 1/3, AAV 1/4, AAV 1/5, AAV 1/6, AAV 1/7, AAV 1/8, AAV1/9, AAV1/10, AAV1/11, AAV1/12, AAV2/1, AAV2/3, AAV2/4, AAV2/5, AAV2/6, AAV2/7, AAV2/8, AAV2/9, AAV2/10, AAV2/11, AAV2/12, AAV3/1, AAV3/2, AAV3/4, AAV3/5, AAV3/6, AAV3/7, AAV3/8, AAV3/9, AAV3/10, AAV3/11, AAV3/12, AAV4/1, AAV4/2, AAV4/3, AAV4/5, AAV4/6, AAV4/7, AAV4/8, AAV4/9, AAV4/10, AAV4/11, AAV4/12, AAV5/1, AAV5/2, AAV5/3, AAV5/4, AAV5/6, AAV5/7, AAV5/8, AAV5/9, AAV5/10, AAV5/11, AAV5/12, AAV6/1, AAV6/2, AAV6/3, AAV6/4, AAV6/5, AAV6/7, AAV6/8, AAV6/9, AAV6/10, AAV6/11, AAV6/12, AAV7/1, AAV7/2, AAV7/3, AAV7/4, AAV7/5, AAV7/6, AAV7/8, AAV7/9, AAV7/10, AAV7/11, AAV7/12, AAV8/1, AAV8/2, AAV8/3, AAV8/4, AAV8/5, AAV8/6, AAV8/7, AAV8/9, AAV8/10, AAV8/11, AAV8/12. AAV9/1, AAV9/2, AAV9/3, AAV9/4, AAV9/5, AAV9/6, AAV9/7, AAV9/8, AAV9/10, AAV9/11, AAV9/12, AAV 10/1, AAV 10/2, AAV 10/3, AAV 10/4, AAV 10/5, AAV 10/6, AAV 10/7, AAV 10/8, AAV 10/9, AAV 10/11, AAV 10/12. AAV 11/1, AAV 11/2, AAV 11/3, AAV 11/4, AAV 11/5, AAV11/6, AAV 11/7, AAV 1/8, AAV 1/9, AAV 1/10, AAV 1/12, AAV 12/1, AAV 12/2, AAV 12/3, AAV 12/4, AAV 12/5, AAV12/6, AAV 12/7, AAV 12/8, AAV 12/9, AAV 12/10, and AAV 12/11. In some embodiments, the AAV comprises a hybrid capsid derived from a plurality of different viral serotypes. For example, in some embodiments, the AAV is AAV-DJ or AAV-DJ8.

In some embodiments, according to any of the virus delivery complexes or nanoparticles described herein, the complex or nanoparticles comprises an AAV as described in Fraser Wright, J., Wellman, J., & High, K. A. Current gene therapy, 10(5): 341-349, 2010; clinical trials NCT02651675, NCT03066258, NCT03003533, NCT02484092, NCT02341807, NCT00999609, NCT01620801, NCT00515710, NCT00516477, and NCT01208389; and patent publications WO2017096039, WO2016179038, and WO2014186579.

In some embodiments, according to any of the virus delivery complexes or nanoparticles described herein, the complex or nanoparticles comprises a recombinant adeno-associated viral (rAAV) vector carrying a human factor VIII (hFVIII) gene. In some embodiments, the rAAV vector is modified to interact more strongly with the human liver than the unmodified rAAV vector. In some embodiments, the virus delivery complexes or nanoparticles are useful for the treatment of hemophilia A.

In some embodiments, according to any of the virus delivery complexes or nanoparticles described herein, the complex or nanoparticles comprises an rAAV vector carrying a human factor IX (hFIX) gene. In some embodiments, the rAAV vector is an rAAV serotype 2 (rAAV2) vector. In some embodiments, the rAAV vector is an rAAV serotype 8 (rAAV8) vector. In some embodiments, the rAAV vector is modified to interact more strongly with the human liver than the unmodified rAAV vector. In some embodiments, the virus delivery complexes or nanoparticles are useful for the treatment of hemophilia B.

In some embodiments, according to any of the virus delivery complexes or nanoparticles described herein, the complex or nanoparticles comprises an rAAV vector carrying a human CHM gene. In some embodiments, the rAAV vector is an rAAV serotype 2 (rAAV2) vector. In some embodiments, the virus delivery complexes or nanoparticles are useful for the treatment of choroideremia (CHM).

In some embodiments, according to any of the virus delivery complexes or nanoparticles described herein, the complex or nanoparticles comprises an rAAV vector carrying a human RPE65 gene. In some embodiments, the rAAV vector is an rAAV serotype 2 (rAAV2) vector. In some embodiments, the virus delivery complexes or nanoparticles are useful for the treatment of Leber Congenital Amaurosis (LCA).

In some embodiments, according to any of the virus delivery complexes or nanoparticles described herein, the complex or nanoparticles comprises an rAAV vector encoding a soluble anti-VEGF protein. In some embodiments, the soluble anti-VEGF protein is a monoclonal antibody fragment which binds to and neutralizes VEGF activity. In some embodiments, the rAAV vector is an rAAV serotype 8 (rAAV8) vector. In some embodiments, the virus delivery complexes or nanoparticles are useful for the treatment of neovascular (wet) age-related macular degeneration (nAMD).

In some embodiments, according to any of the virus delivery complexes or nanoparticles described herein, the complex or nanoparticles comprises an rAAV vector carrying a human low-density lipoprotein receptor (LDLR) gene. In some embodiments, the rAAV vector is an rAAV serotype 8 (rAAV8) vector. In some embodiments, the rAAV vector is modified to interact more strongly with the human liver than the unmodified rAAV vector. In some embodiments, the virus delivery complexes or nanoparticles are useful for the treatment of homozygous familial hypercholesterolemia (HoFH).

In some embodiments, according to any of the virus delivery complexes or nanoparticles described herein, the complex or nanoparticles comprises an rAAV vector carrying a human α-1-iduronidase (IDUA) gene. In some embodiments, the rAAV vector is an rAAV serotype 9 (rAAV9) vector. In some embodiments, the rAAV vector is modified to interact more strongly with the human liver than the unmodified rAAV vector. In some embodiments, the virus delivery complexes or nanoparticles are useful for the treatment of Mucopolysaccharidosis Type I (MPS I).

In some embodiments, according to any of the virus delivery complexes or nanoparticles described herein, the complex or nanoparticles comprises an rAAV vector carrying a human iduronate-2-sulfatase (IDS) gene. In some embodiments, the rAAV vector is an rAAV serotype 9 (rAAV9) vector. In some embodiments, the rAAV vector is modified to interact more strongly with the human liver than the unmodified rAAV vector. In some embodiments, the virus delivery complexes or nanoparticles are useful for the treatment of Mucopolysaccharidosis Type II (MPS II).

### Adenovirus

In some embodiments, according to any of the virus delivery complexes or nanoparticles described herein, the complex or nanoparticles comprises an adenovirus. In some embodiments, the adenovirus is any one of Ad1-Ad57. In some embodiments, the adenovirus is Ad5.

### Lentivirus

In some embodiments, according to any of the virus delivery complexes or nanoparticles described herein, the complex or nanoparticles comprises a lentivirus.

### Retrovirus

In some embodiments, according to any of the virus delivery complexes or nanoparticles described herein, the complex or nanoparticles comprises a retrovirus. In some embodiments, the retrovirus is a γ-retrovirus.

### Herpes Simplex Virus (HSV)

In some embodiments, according to any of the virus delivery complexes or nanoparticles described herein, the complex or nanoparticles comprises a herpes simplex virus (HSV). In some embodiments, the HSV is HSV-1. In some embodiments, the HSV is HSV-2.

### Poxvirus

In some embodiments, according to any of the virus delivery complexes or nanoparticles described herein, the complex or nanoparticles comprises a poxvirus.

### Epstein-Barr Virus (EBV)

In some embodiments, according to any of the virus delivery complexes or nanoparticles described herein, the complex or nanoparticles comprises an Epstein-Barr virus (EBV).

### Vaccinia Virus

In some embodiments, according to any of the virus delivery complexes or nanoparticles described herein, the complex or nanoparticles comprises a vaccinia virus.

Human Cytomegalovirus (hCMV)

In some embodiments, according to any of the virus delivery complexes or nanoparticles described herein, the complex or nanoparticles comprises a cytomegalovirus (CMV). In some embodiments, the CMV is human CMV (hCMV).

### Compositions

In some embodiments, there is provided a composition comprising a virus delivery complex or nanoparticle as described herein. In some embodiments, the composition is a pharmaceutical composition comprising a virus delivery complex or nanoparticle as described herein and a pharmaceutically acceptable diluent, excipient and/or carrier. In some embodiments, the concentration of the complex or nanoparticle in the composition is from about 1 nM to about 100 mM, including for example from about 10 nM to about 50 mM, from about 25 nM to about 25 mM, from about 50 nM to about 10 mM, from about 100 nM to about 1 mM, from about 500 nM to about 750 μM, from about 750 nM to about 500 μM, from about 1 μM to about 250 μM, from about 10 μM to about 200 μM, and from about 50 μM to about 150 μM.

The term “pharmaceutically acceptable diluent, excipient, and/or carrier” as used herein is intended to include any and all solvents, dispersion media, coatings, antibacterial and antifungal agents, isotonic and absorption delaying agents, and the like, compatible with administration to humans or other vertebrate hosts. Typically, a pharmaceutically acceptable diluent, excipient, and/or carrier is a diluent, excipient, and/or carrier approved by a regulatory agency of a Federal, a state government, or other regulatory agency or listed in the U.S. Pharmacopeia or other generally recognized pharmacopeia for use in animals, including humans as well as non-human mammals. The term diluent, excipient, and/or “carrier” refers to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like, including lyophilization aids. The composition, if desired, can also contain minor amounts of wetting, bulking, emulsifying agents, or pH buffering agents. These compositions can take the form of solutions, suspensions, emulsion, sustained release formulations and the like. Examples of suitable pharmaceutical diluent, excipient, and/or carriers are described in “Remington's Pharmaceutical Sciences” by E. W. Martin. The formulation should suit the mode of administration. The appropriate diluent, excipient, and/or carrier will be evident to those skilled in the art and will depend in large part upon the route of administration.

In some embodiments, a pharmaceutical composition as described herein is formulated for intravenous, intratumoral, intraarterial, topical, intraocular, ophthalmic, intraportal, intracranial, intracerebral, intracerebroventricular, intrathecal, intravesicular, intradermal, subcutaneous, intramuscular, intranasal, intratracheal, pulmonary, intracavity, or oral administration.

In some embodiments, dosages of the pharmaceutical compositions of the present invention found to be suitable for treatment of human or mammalian subjects are in the range of about 0.001 mg/kg to about 100 mg/kg (such as about any of 0.001, 0.01, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, and 100 mg/kg, including any ranges between these values) of the virus delivery complexes or nanoparticles. In some embodiments, dosage ranges are about 0.1 mg/kg to about 20 mg/kg (such as about any of 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, and 20 mg/kg, including any ranges between these values). In some embodiments, dosage ranges are about 0.5 mg/kg to about 10 mg/kg.

Exemplary dosing frequencies include, but are not limited to, weekly without break: weekly, three out of four weeks; once every three weeks; once every two weeks; weekly, two out of three weeks. In some embodiments, the pharmaceutical composition is administered about once every 2 weeks, once every 3 weeks, once every 4 weeks, once every 6 weeks, or once every 8 weeks. In some embodiments, the pharmaceutical composition is administered at least about any of 1×, 2×, 3×, 4×, 5×, 6×, or 7× (i.e., daily) a week. In some embodiments, the intervals between each administration are less than about any of 6 months, 3 months, 1 month, 20 days, 15, days, 12 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week. In some embodiments, the schedule of administration of the pharmaceutical composition to an individual ranges from a single administration that constitutes the entire treatment to daily administration. The administration of the pharmaceutical composition can be extended over an extended period of time, such as from about a month up to about seven years. In some embodiments, the pharmaceutical composition is administered over a period of at least about any of 2, 3, 4, 5, 6, 7, 8, 9, 10, 11, 12, 18, 24, 30, 36, 48, 60, 72, or 84 months.

In some embodiments, there is provided a pharmaceutical composition comprising a virus delivery complex or nanoparticle as described herein and a pharmaceutically acceptable carrier, wherein the pharmaceutically acceptable carrier is a sugar or a protein. In some embodiments, the sugar is selected from the group consisting of sucrose, glucose, mannitol, and a combination thereof, and is present in the pharmaceutical composition at a concentration from about 5% to about 20%. In some embodiments, the sugar is sucrose. In some embodiments, the sugar is glucose. In some embodiments, the sugar is mannitol. In some embodiments, the protein is albumin. In some embodiments, the albumin is human serum albumin. In some embodiments, the pharmaceutical composition is lyophilized.

### Methods of Preparation

In some embodiments, there is provided a method of preparing a virus delivery complex or nanoparticle as described herein comprising combining a CPP with one or more viruses, thereby forming the virus delivery complex or nanoparticle.

Thus, in some embodiments, there is provided a method of preparing a virus delivery complex or nanoparticle as described herein comprising combining a CPP with one or more viruses.

For example, in some embodiments, there is provided a method of preparing a virus delivery complex or nanoparticle as described herein comprising a) combining a first composition comprising one or more viruses with a second composition comprising a cell-penetrating peptide in an aqueous medium to form a mixture; and b) incubating the mixture to form a complex comprising the cell-penetrating peptide associated with the one or more viruses, thereby generating the virus delivery complex or nanoparticle. In some embodiments, the aqueous medium is a buffer, including for example PBS. Tris, or any buffer known in the art for stabilizing protein complexes. In some embodiments, the first composition comprising the one or more viruses is a solution comprising at least one of the one or more viruses (such as an AAV, a lentivirus, and/or a herpesvirus) at a titer from about 1×10⁴ to about 1×10¹⁵ (such as from about 1×10⁷ to about 1×10¹² or from about 1×10⁹ to about 1×10¹¹). In some embodiments, the first composition comprising the one or more viruses is a solid comprising the one or more viruses in lyophilized form and a suitable carrier. In some embodiments, the second composition comprising the cell-penetrating peptide is a solution comprising the cell-penetrating peptide at a concentration from about 1 nM to about 200 μM (such as about any of 2 nM, 5 nM, 10 nM, 25 nM, 50 nM, 100 nM, 200 nM, 300 nM, 400 nM, 500 nM, 600 nM, 700 nM, 800 nM, 900 nM, 1 μM, 2 μM, 5 μM, 10 μM, 25 μM, 50 μM, 100 μM, 150 μM, or 200 μM, including any ranges between these values). In some embodiments, the second composition comprising the cell-penetrating peptide is a solid comprising the cell-penetrating peptide in lyophilized form and a suitable carrier. In some embodiments, the solutions are formulated in water. In some embodiments, the water is distilled water. In some embodiments, the solutions are formulated in a buffer. In some embodiments, the buffer is any buffer known in the art used for storing a virus or polypeptide. In some embodiments, the molar ratio of the cell-penetrating peptide to virus (e.g., in Vg, pfu, or MOI) associated with the cell-penetrating peptide in the mixture is between about 1:1 and about 1×10⁸:1. In some embodiments, the mixture is incubated to form a complex or nanoparticle comprising the cell-penetrating peptide associated with the one or more viruses for from about 10 min to 60 min, including for example for about any of 20 min, 30 min, 40 min, and 50 min, at a temperature from about 2° C. to about 50° C., including for example from about 2° C. to about 5° C., from about 5° C. to about 10° C., from about 10° C. to about 15° C., from about 15° C. to about 20° C., from about 20° C. to about 25° C., from about 25° C. to about 30° C., from about 30° C. to about 35° C., from about 35° C. to about 40° C., from about 40° C. to about 45° C., and from about 45° C. to about 50° C., thereby resulting in a solution comprising the virus delivery complex or nanoparticle. In some embodiments, the solution comprising the virus delivery complex or nanoparticle remains stable for at least about three weeks, including for example for at least about any of 6 weeks, 2 months, 3 months, 4 months, 5 months, and 6 months at 4° C. In some embodiments, the solution comprising the virus delivery complex or nanoparticle is lyophilized in the presence of a carrier. In some embodiments, the carrier is a sugar, including for example, sucrose, glucose, mannitol and combinations thereof, and is present in the solution comprising the virus delivery complex or nanoparticle at from about 5% to about 20%, including for example from about 7.5% to about 17.5%, from about 10% to about 15%, and about 12.5%, weight per volume. In some embodiments, the carrier is a protein, including for example albumin, such as human serum albumin. In some embodiments, the cell-penetrating peptide is a PEP-1 peptide, a PEP-2 peptide, a VEPEP-3 peptide, a VEPEP-6 peptide, a VEPEP-9 peptide, or an ADGN-100 peptide as described herein. In some embodiments, the cell-penetrating peptide comprises the amino acid sequence of any one of SEQ ID NOs: 75-80.

In some embodiments, there is provided a method of preparing a nanoparticle comprising a core and at least one additional layer as described herein, comprising a) combining a composition comprising one or more viruses with a composition comprising a first cell-penetrating peptide in an aqueous medium to form a first mixture; b) incubating the first mixture to form a core of the nanoparticle comprising the first cell-penetrating peptide associated with the one or more viruses; c) combining a composition comprising the core of the nanoparticle, such as the mixture of b), with a composition comprising a second cell-penetrating peptide in an aqueous medium to form a second mixture, and d) incubating the second mixture to form a nanoparticle comprising a core and at least one additional layer. In some embodiments, the method further comprises e) combining a composition comprising the nanoparticle comprising a core and at least one additional layer and a composition comprising a third cell-penetrating peptide in an aqueous medium to form a third mixture, and f) incubating the third mixture to form a nanoparticle comprising a core and at least two additional layers. It is to be appreciated that the method can be adapted to form a nanoparticle comprising increasing numbers of layers. In some embodiments, the aqueous medium is a buffer, including for example PBS, Tris, or any buffer known in the art for stabilizing protein complexes. In some embodiments, the composition comprising the one or more viruses is a solution comprising the one or more viruses at a titer from about 1×10⁴ to about 1×10¹⁵ (such as from about 1×10⁷ to about 1×10¹² or from about 1×10⁹ to about 1×10¹¹). In some embodiments, the composition comprising the one or more viruses is a solid comprising the one or more viruses in lyophilized form and a suitable carrier. In some embodiments, the compositions comprising the first, second, and/or third cell-penetrating peptides are each a solution comprising the cell-penetrating peptide at a concentration from about 1 nM to about 200 μM (such as about any of 2 nM, 5 nM, 10 nM, 25 nM, 50 nM, 100 nM, 200 nM, 300 nM, 400 nM, 500 nM, 600 nM, 700 nM, 800 nM, 900 nM, 1 μM, 2 μM, 5 μM, 10 μM, 25 μM, 50 μM, 100 μM, 150 μM, or 200 μM, including any ranges between these values). In some embodiments, the compositions comprising the first, second, and/or third cell-penetrating peptides are each a solid comprising the cell-penetrating peptide in lyophilized form and a suitable carrier. In some embodiments, the solutions are formulated in water. In some embodiments, the water is distilled water. In some embodiments, the solutions are formulated in a buffer. In some embodiments, the buffer is any buffer known in the art used for storing a virus or polypeptide. In some embodiments, the molar ratio of the first cell-penetrating peptide to virus (e.g., in Vg, pfu, or MOI) in the first mixture is between about 1:1 and about 1×10⁸: 1. In some embodiments, the first, second, and/or third mixtures are individually incubated for from about 10 min to 60 min, including for example for about any of 20 min, 30 min, 40 min, and 50 min, at a temperature from about 2° C. to about 50° C., including for example from about 2° C. to about 5° C., from about 5° C. to about 10° C., from about 10° C. to about 15° C., from about 15° C. to about 20° C. from about 20° C. to about 25° C., from about 25° C. to about 30° C., from about 30° C. to about 35° C., from about 35° C. to about 40° C., from about 40° C. to about 45° C., and from about 45° C. to about 50° C. In some embodiments, the solution comprising the nanoparticle remains stable for at least about three weeks, including for example for at least about any of 6 weeks, 2 months, 3 months, 4 months, 5 months, and 6 months at 4° C. In some embodiments, the solution comprising the nanoparticle is lyophilized in the presence of a carrier. In some embodiments, the carrier is a sugar, including for example, sucrose, glucose, mannitol and combinations thereof, and is present in the solution comprising the virus delivery complex or nanoparticle at from about 5% to about 20%, including for example from about 7.5% to about 17.5%, from about 10% to about 15%, and about 12.5%, weight per volume. In some embodiments, the carrier is a protein, including for example albumin, such as human serum albumin. In some embodiments, the first, second, and/or third cell-penetrating peptides are individually a PEP-1 peptide, a PEP-2 peptide, a VEPEP-3 peptide, a VEPEP-6 peptide, a VEPEP-9 peptide, or an ADGN-100 peptide as described herein. In some embodiments, the first, second, and/or third cell-penetrating peptides individually comprises the amino acid sequence of SEQ ID NO: 75, 76, 77, 78, 79, or 80.

In some embodiments, for a stable composition comprising a virus delivery complex or nanoparticle of the invention, the average diameter of the complex or nanoparticle does not change by more than about 10%, and the polydispersity index does not change by more than about 10%.

Also provided are methods of preparing any of the CPPs described herein.

### Methods of Use

### Methods of Disease Treatment

In some embodiments, there is provided a method of treating a disease or condition in an individual comprising administering to the individual an effective amount of a pharmaceutical composition comprising a virus delivery complex or nanoparticle as described herein for intracellular delivery of a virus and a pharmaceutically acceptable carrier, wherein the virus delivery complex or nanoparticle comprises one or more viruses useful for the treatment of the disease or condition. In some embodiments, the virus delivery complex or nanoparticle comprises a CPP comprising the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the lowest effective amount of virus in the pharmaceutical composition is less than the lowest effective amount of virus in a similar pharmaceutical composition where the virus is not in a virus delivery complex or nanoparticle as described herein (e.g., a pharmaceutical composition comprising free virus). In some embodiments, the virus is a recombinant virus, including recombinant AAV, adenovirus, lentivirus, retrovirus, HSV, poxvirus, EBV, vaccinia virus, and hCMV. In some embodiments, the recombinant virus comprises a transgene for insertion into a cell genome. In some embodiments, the transgene is a therapeutic transgene. In some embodiments, the transgene encodes a protein, such as a therapeutic protein. In some embodiments, the transgene encodes an inhibitory RNA (RNAi), such as an RNAi targeting an endogenous gene, e.g., a disease-associated endogenous gene. In some embodiments, the transgene encodes a CAR. In some embodiments, the complex or nanoparticle comprises one or more viruses comprising a first transgene encoding an RNAi and a second transgene encoding a protein. In some embodiments, the RNAi is a therapeutic RNAi targeting an endogenous gene involved in a disease or condition, and the protein is a therapeutic protein useful for treating the disease or condition. In some embodiments, the therapeutic RNAi targets a disease-associated form of the endogenous gene (e.g., a gene encoding a mutant protein, or a gene resulting in abnormal expression of a protein), and the second transgene is a therapeutic form of the endogenous gene (e.g., the second transgene encodes a wild-type or functional form of the mutant protein, or the second transgene results in normal expression of the protein). In some embodiments, the complex or nanoparticle comprises a first virus comprising the first transgene and a second virus comprising the second transgene. In some embodiments, the complex or nanoparticle comprises a single virus comprising the first transgene and the second transgene. In some embodiments, the disease or condition to be treated includes, but is not limited to, cancer, diabetes, autoimmune diseases, inflammatory diseases, fibrotic diseases, viral infectious diseases, hereditary diseases, ocular diseases, aging and degenerative diseases, and diseases characterized by cholesterol level abnormality. In some embodiments, the virus is capable of modifying the sequence of one or more genes. In some embodiments, the virus is capable of modulating the expression of one or more genes. In some embodiments, the one or more genes encode proteins including, but not limited to, growth factors and cytokines, cell surface receptors, signaling molecules and kinases, transcription factors and other modulators of transcription, regulators of protein expression and modification, tumor suppressors, and regulators of apoptosis and metastasis. In some embodiments, the pharmaceutical composition further comprises one or more additional virus delivery complexes or nanoparticles as described herein. In some embodiments, the method further comprises administering to the individual an effective amount of one or more additional pharmaceutical compositions comprising one or more additional virus delivery complexes or nanoparticles as described herein. In some embodiments, the target polynucleotide is modified to inactivate a target gene, such as by decreasing expression of the target gene or resulting in a modified target gene that expresses an inactive product. In some embodiments, the target polynucleotide is modified to activate a target gene, such as by increasing expression of the target gene or resulting in a modified target gene that expresses an active target gene product.

“Modulation” of activity or expression used herein means regulating or altering the status or copy numbers of a gene or mRNA or changing the amount of gene product such as a protein that is produced. In some embodiments, the virus inhibits the expression of a target gene. In some embodiments, the virus inhibits the expression of the gene or gene product by at least about any of 0%, 20%, 30%, 40%, 60%, 70%, 80%, 90%, or 100%.

In some embodiments, there is provided a method of treating a disease or condition in an individual comprising administering to the individual an effective amount of a pharmaceutical composition comprising a virus delivery complex or nanoparticle as described herein for intracellular delivery of a virus and a pharmaceutically acceptable carrier, wherein the virus delivery complex or nanoparticle comprises one or more viruses useful for the treatment of the disease or condition and a cell-penetrating peptide comprising the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the disease or condition to be treated includes, but is not limited to, cancer, diabetes, autoimmune diseases, inflammatory diseases, fibrotic diseases, viral infectious diseases, hereditary diseases, ocular diseases, aging and degenerative diseases, and cholesterol level abnormality. In some embodiments, the virus delivery complex or nanoparticle in the pharmaceutical composition comprises one or more viruses for modifying the sequence of one or more genes in the individual. In some embodiments, the virus delivery complex or nanoparticle in the pharmaceutical composition comprises one or more viruses for modulating the expression of one or more genes in the individual. In some embodiments, the one or more genes encode proteins including, but not limited to, growth factors and cytokines, cell surface receptors, signaling molecules and kinases, transcription factors and other modulators of transcription, regulators of protein expression and modification, tumor suppressors, and regulators of apoptosis and metastasis. In some embodiments, the pharmaceutical composition further comprises one or more additional virus delivery complexes or nanoparticles as described herein. In some embodiments, the method further comprises administering to the individual an effective amount of one or more additional pharmaceutical compositions comprising one or more additional virus delivery complexes or nanoparticles as described herein. In some embodiments, the target polynucleotide is modified to inactivate a target gene, such as by decreasing expression of the target gene or resulting in a modified target gene that expresses an inactive product. In some embodiments, the target polynucleotide is modified to activate a target gene, such as by increasing expression of the target gene or resulting in a modified target gene that expresses an active target gene product.

In some embodiments of the methods described herein, the virus delivery complex or nanoparticle comprises one or more viruses comprising one or more transgenes (such as about any of 1, 2, 3, 4, 5, or more transgenes). In some embodiments, one or more of the transgenes encode a protein, such as a therapeutic protein. In some embodiments, one or more of the transgenes encode an inhibitory RNA (RNAi), such as a therapeutic RNAi. In some embodiments, one or more of the transgenes encode a chimeric antigen receptor (CAR).

In some embodiments, there is provided a method of treating a disease or condition in an individual comprising administering to the individual an effective amount of a pharmaceutical composition comprising a virus delivery complex or nanoparticle as described herein and a pharmaceutically acceptable carrier, wherein the pharmaceutical composition is less immunogenic than a similar pharmaceutical composition comprising the one or more viruses contained in the virus delivery complex or nanoparticle alone (i.e., a pharmaceutical composition comprising the one or more viruses not associated with a peptide as described herein). In some embodiments, the pharmaceutical compositions is no more than about 99% (such as no more than about any of 95, 90, 85, 80, 75, 70, 65, 60, 55, 50, 45, 40, 35, 30, 25, 20, 15, 10, 5, 4, 3, 2, 1% or less, including any ranges between these values) as immunogenic as a similar pharmaceutical composition comprising the one or more viruses contained in the virus delivery complex or nanoparticle alone.

In some embodiments, there is provided a method of treating a disease or condition in an individual comprising administering to the individual an effective amount of a pharmaceutical composition comprising a virus delivery complex or nanoparticle as described herein and a pharmaceutically acceptable carrier, wherein the method comprises multiple administrations of the pharmaceutical composition. In some embodiments, repeated administrations of the pharmaceutical compositions do not elicit an adverse immune response in the individual to the pharmaceutical composition, or elicit a substantially reduced immune response in the individual compared to repeated administrations of a similar pharmaceutical composition comprising the one or more viruses contained in the virus delivery complex or nanoparticle alone. In some embodiments, a repeated administration of the pharmaceutical compositions results in an immune response no more than about 99% (such as no more than about any of 95, 90, 85, 80, 75, 70, 65, 60, 55, 50, 45, 40, 35, 30, 25, 20, 15, 10, 5, 4, 3, 2, 1% or less, including any ranges between these values) as strong as the immune response generated by a corresponding repeated administration of a similar pharmaceutical composition comprising the one or more viruses contained in the virus delivery complex or nanoparticle alone.

In some embodiments, there is provided a method of treating a disease or condition in an individual comprising administering to the individual an effective amount of a pharmaceutical composition comprising a virus delivery complex or nanoparticle as described herein and a pharmaceutically acceptable carrier, wherein the individual produces neutralizing antibodies to at least one of the one or more viruses contained in the virus delivery complex or nanoparticle, and the peptides of the virus delivery complex or nanoparticle mask the at least one virus from the neutralizing antibodies. In some embodiments, the neutralizing antibodies are blocked from neutralizing the at least one virus in the complex or nanoparticle, or result in substantially reduced neutralizing of the at least one virus in the complex or nanoparticle compared to the at least one virus alone (i.e., the at least one virus not associated with a peptide as described herein). In some embodiments, neutralization of the at least one virus in the complex or nanoparticle by the neutralizing antibodies is no more than about 99% (such as no more than about any of 95, 90, 85, 80, 75, 70, 65, 60, 55, 50, 45, 40, 35, 30, 25, 20, 15, 10, 5, 4, 3, 2, 1% or less, including any ranges between these values) of the neutralization of the at least one virus alone by the neutralizing antibodies.

### Diseases and Conditions

In some embodiments of the methods described herein, the disease to be treated is cancer. In some embodiments, the cancer is a solid tumor, and the pharmaceutical composition comprises a virus delivery complex or nanoparticle comprising one or more viruses that modulates the expression of one or more genes that encode proteins including, but not limited to, growth factors and cytokines, cell surface receptors, signaling molecules and kinases, transcription factors and other modulators of transcription, regulators of protein expression and modification, tumor suppressors, and regulators of apoptosis and metastasis. In some embodiments, the growth factors or cytokines include, but are not limited to, EGF, VEGF, FGF, HGF, HDGF, IGF, PDGF, TGF-α, TGF-β, TNF-α, and wnt, including mutants thereof. In some embodiments, the cell surface receptors include, but are not limited to, ER, PR, Her2, Her3, angiopoietin receptor, EGFR, FGFR, HGFR, HDGFR, IGFR, KGFR, MSFR, PDGFR, TGFR, VEGFR1, VEGFR2, VEGFR3, Frizzled family receptors (FZD-1 to 10), smoothened, patched, and CXCR4, including mutants thereof. In some embodiments, the signaling molecules or kinases include, but are not limited to, KRAS, NRAS, RAF, MEK, MEKK, MAPK, MKK, ERK, JNK, JAK, PKA, PKC, PI3K, Akt, mTOR, Raptor, Rictor, MLST8, PRAS40, DEPTOR, MSIN1, S6 kinase, PDK1, BRAF, FAK, Src, Fyn, She, GSK, IKK, PLK-1, cyclin-dependent kinases (Cdk1 to 13), CDK-activating kinases, ALK/Met, Syk, BTK, Bcr-Abl, RET, β-catenin, Mcl-1, and PKN3, including mutants thereof. In some embodiments, the transcription factors or other modulators of transcription include, but are not limited to, AR, ATF1, CEBPA, CREB1, ESR1, EWSR1, FOXO1, GATA 1, GATA3, HNF1A, HNF1B, IKZF1, IRF1, IRF4, KLF6, LMO1, LYL1, MYC, NR4A3, PAX3, PAX5, PAX7, PBX 1, PHOX2B, PML, RUNX1, SMAD4, SMAD7, STAT5B, TAL1, TP53, WT1, ZBTB16, ATF-2, Chop, c-Jun, c-Myc, DPC4, Elk-1, Ets1, Max, MEF2C, NFAT4, Sap1a, STATs, Tal, p53, CREB, NF-κB, HDACs, HIF-1α, and RRM2, including mutants thereof. In some embodiments, the regulators of protein expression or modification include, but are not limited to, ubiquitin ligase. LMP2, LMP7, and MECL-1, including mutants thereof. In some embodiments, the tumor suppressors include, but are not limited to, APC, BRCA1, BRCA2, DPC4, INK4, MADR2, MLH1, MSH2, MSH6, NF1, NF2, p53, PTC, PTEN, Rb, VHL, WT1, and WT2, including mutants thereof. In some embodiments, the regulators of apoptosis or metastasis include, but are not limited to, XIAP, Bcl-2, osteopontin, SPARC, MMP-2, MMP-9, uPAR, including mutants thereof.

In some embodiments of the methods described herein, the disease to be treated is cancer, wherein the cancer is a solid tumor, and the pharmaceutical composition comprises a virus delivery complex or nanoparticle comprising one or more viruses targeting one or more genes encoding proteins involved in tumor development and/or progression. In some embodiments, the one or more genes encoding proteins involved in tumor development and/or progression include, but are not limited to, IL-2, IL-12, interferon-gamma, GM-CSF, B7-1, caspase-9, p53, MUC-1, MDR-1, HLA-B7/Beta 2-Microglobulin, Her2, Hsp27, thymidine kinase, and MDA-7, including mutants thereof. In some embodiments, the virus is a recombinant virus, including recombinant AAV, adenovirus, lentivirus, retrovirus, HSV, poxvirus, EBV, vaccinia virus, and hCMV. In some embodiments, the recombinant virus comprises a transgene, and intracellular delivery of the virus allows for transfer of the transgene into the genome of the cell. In some embodiments, the transgene is a therapeutic transgene. In some embodiments, the transgene encodes a protein, such as a therapeutic protein. In some embodiments, the transgene encodes an inhibitory RNA (RNAi), such as an RNAi targeting an endogenous gene, e.g., a disease-associated endogenous gene. In some embodiments, the transgene encodes a CAR. In some embodiments, the complex or nanoparticle comprises one or more viruses comprising a first transgene encoding an RNAi and a second transgene encoding a protein. In some embodiments, the RNAi is a therapeutic RNAi targeting an endogenous gene involved in a disease or condition, and the protein is a therapeutic protein useful for treating the disease or condition. In some embodiments, the therapeutic RNAi targets a disease-associated form of the endogenous gene (e.g., a gene encoding a mutant protein, or a gene resulting in abnormal expression of a protein), and the second transgene is a therapeutic form of the endogenous gene (e.g., the second transgene encodes a wild-type or functional form of the mutant protein, or the second transgene results in normal expression of the protein). In some embodiments, the complex or nanoparticle comprises a first virus comprising the first transgene and a second virus comprising the second transgene. In some embodiments, the complex or nanoparticle comprises a single virus comprising the first transgene and the second transgene.

In some embodiments of the methods described herein, the disease to be treated is cancer, wherein the cancer is liver cancer, and the pharmaceutical composition comprises a virus delivery complex or nanoparticle comprising one or more viruses targeting one or more genes encoding proteins involved in liver cancer development and/or progression. In some embodiments, the liver cancer is hepatocellular carcinoma, cholangiocarcinoma, angiosarcoma of the liver, or hemangiosarcoma of the liver. In some embodiments, the one or more genes encoding proteins involved in liver cancer development and/or progression include, but are not limited to, CCND2, RAD23B, GRP78, CEP164, MDM2, and ALDH2, including mutants thereof.

In some embodiments, according to any of the methods described herein, the cancer is hepatocellular carcinoma (HCC). In some embodiments, the HCC is early stage HCC, non-metastatic HCC, primary HCC, advanced HCC, locally advanced HCC, metastatic HCC, HCC in remission, or recurrent HCC. In some embodiments, the HCC is localized resectable (i.e., tumors that are confined to a portion of the liver that allows for complete surgical removal), localized unresectable (i.e., the localized tumors may be unresectable because crucial blood vessel structures are involved or because the liver is impaired), or unresectable (i.e., the tumors involve all lobes of the liver and/or has spread to involve other organs (e.g., lung, lymph nodes, bone). In some embodiments, the HCC is, according to TNM classifications, a stage I tumor (single tumor without vascular invasion), a stage II tumor (single tumor with vascular invasion, or multiple tumors, none greater than 5 cm), a stage III tumor (multiple tumors, any greater than 5 cm, or tumors involving major branch of portal or hepatic veins), a stage IV tumor (tumors with direct invasion of adjacent organs other than the gallbladder, or perforation of visceral peritoneum), N1 tumor (regional lymph node metastasis), or MI tumor (distant metastasis). In some embodiments, the HCC is, according to AJCC (American Joint Commission on Cancer) staging criteria, stage T1, T2, T3, or T4 HCC. In some embodiments, the HCC is any one of liver cell carcinomas, fibrolamellar variants of HCC, and mixed hepatocellular cholangiocarcinomas. In some embodiments, the individual may be a human who has a gene, genetic mutation, or polymorphism associated with hepatocellular carcinoma (e.g., mutation or polymorphism in CCND2, RAD23B, GRP78, CEP164, MDM2, and/or ALDH2) or has one or more extra copies of a gene associated with hepatocellular carcinoma.

In some embodiments of the methods described herein, the disease to be treated is cancer, wherein the cancer is lung cancer, and the pharmaceutical composition comprises a virus delivery complex or nanoparticle comprising one or more viruses targeting one or more genes encoding proteins involved in lung cancer development and/or progression. In some embodiments, the one or more genes encoding proteins involved in lung cancer development and/or progression include, but are not limited to, SASH1, LATS1, IGF2R, PARK2, KRAS, PTEN, Kras2, Krag, Pas1, ERCC1, XPD, IL8RA, EGFR, Ot₁-AD, EPHX, MMP1, MMP2, MMP3, MMP12, IL1β, RAS, and AKT, including mutants thereof.

In some embodiments, according to any of the methods described herein, the cancer is lung cancer. In some embodiments, the lung cancer is a non-small cell lung cancer (NSCLC). Examples of NSCLC include, but are not limited to, large-cell carcinoma (e.g., large-cell neuroendocrine carcinoma, combined large-cell neuroendocrine carcinoma, basaloid carcinoma, lymphoepithelioma-like carcinoma, clear cell carcinoma, and large-cell carcinoma with rhabdoid phenotype), adenocarcinoma (e.g., acinar, papillary (e.g., bronchioloalveolar carcinoma, nonmucinous, mucinous, mixed mucinous and nonmucinous and indeterminate cell type), solid adenocarcinoma with mucin, adenocarcinoma with mixed subtypes, well-differentiated fetal adenocarcinoma, mucinous (colloid) adenocarcinoma, mucinous cystadenocarcinoma, signet ring adenocarcinoma, and clear cell adenocarcinoma), neuroendocrine lung tumors, and squamous cell carcinoma (e.g., papillary, clear cell, small cell, and basaloid). In some embodiments, the NSCLC is, according to TNM classifications, a stage T tumor (primary tumor), a stage N tumor (regional lymph nodes), or a stage M tumor (distant metastasis). In some embodiments, the lung cancer is a carcinoid (typical or atypical), adenosquamous carcinoma, cylindroma, or carcinoma of the salivary gland (e.g., adenoid cystic carcinoma or mucoepidermoid carcinoma). In some embodiments, the lung cancer is a carcinoma with pleomorphic, sarcomatoid, or sarcomatous elements (e.g., carcinomas with spindle and/or giant cells, spindle cell carcinoma, giant cell carcinoma, carcinosarcoma, or pulmonary blastoma). In some embodiments, the cancer is small cell lung cancer (SCLC; also called oat cell carcinoma). The small cell lung cancer may be limited-stage, extensive stage or recurrent small cell lung cancer. In some embodiments, the individual may be a human who has a gene, genetic mutation, or polymorphism suspected or shown to be associated with lung cancer (e.g., mutation or polymorphism in SASH1, LATS1, IGF2R, PARK2, KRAS, PTEN, Kras2, Krag, Pas1, ERCC1, XPD, IL8RA, EGFR, Ot₁-AD, EPHX, MMP1, MMP2, MMP3, MMP12, IL1β, RAS, and/or AKT) or has one or more extra copies of a gene associated with lung cancer.

In some embodiments of the methods described herein, the disease to be treated is cancer, wherein the cancer is renal cell carcinoma (RCC), and the pharmaceutical composition comprises a virus delivery complex or nanoparticle comprising one or more viruses targeting one or more genes encoding proteins involved in RCC development and/or progression. In some embodiments, the one or more genes encoding proteins involved in RCC development and/or progression include, but are not limited to, VHL, TSC1, TSC2, CUL2, MSH2, MLH1, INK4a/ARF, MET, TGF-σ, TGF-β1, IGF-I, IGF-IR, AKT, and PTEN, including mutants thereof.

In some embodiments, according to any of the methods described above, the cancer is renal cell carcinoma. In some embodiments, the renal cell carcinoma is an adenocarcinoma. In some embodiments, the renal cell carcinoma is a clear cell renal cell carcinoma, papillary renal cell carcinoma (also called chromophilic renal cell carcinoma), chromophobe renal cell carcinoma, collecting duct renal cell carcinoma, granular renal cell carcinoma, mixed granular renal cell carcinoma, renal angiomyolipomas, or spindle renal cell carcinoma. In some embodiments, the individual may be a human who has a gene, genetic mutation, or polymorphism associated with renal cell carcinoma (e.g., mutation or polymorphism in VHL, TSC1, TSC2, CUL2, MSH2, MLH1, INK4a/ARF, MET, TGF-α, TGF-β1, IGF-I, IGF-IR, AKT, and/or PTEN) or has one or more extra copies of a gene associated with renal cell carcinoma. In some embodiments, the renal cell carcinoma is associated with (1) von Hippel-Lindau (VHL) syndrome. (2) hereditary papillary renal carcinoma (HPRC), (3) familial renal oncocytoma (FRO) associated with Birt-Hogg-Dube syndrome (BHDS), or (4) hereditary renal carcinoma (HRC). In some embodiments, the renal cell carcinoma is at any of stage I, II, III, or IV, according to the American Joint Committee on Cancer (AJCC) staging groups. In some embodiments, the renal cell carcinoma is stage IV renal cell carcinoma.

In some embodiments of the methods described herein, the disease to be treated is cancer, wherein the cancer is a central nervous system (CNS) tumor, and the pharmaceutical composition comprises a virus delivery complex or nanoparticle comprising one or more viruses targeting one or more genes encoding proteins involved in the CNS tumor development and/or progression. In some embodiments, the pharmaceutical composition is administered during or after (such as immediately following) a surgical procedure on the CNS tumor. In some embodiments, the surgical procedure is resection of the CNS tumor. In some embodiments, the pharmaceutical composition is administered into a surgical cavity resulting from the surgical procedure. In some embodiments, the one or more genes encoding proteins involved in the CNS tumor development and/or progression include, but are not limited to, NF1, NF2, SMARCB1, pVHL, TSC1, TSC2, p53, CHK2, MLH1, PMS2, PTCH, SUFU, and XRCC7, including mutants thereof.

In some embodiments, according to any of the methods described herein, the cancer is a CNS tumor. In some embodiments, the CNS tumor is a glioma (e.g., brainstem glioma and mixed gliomas), glioblastoma (also known as glioblastoma multiforme), astrocytoma (such as high-grade astrocytoma), pediatric glioma or glioblastoma (such as pediatric high-grade glioma (HGG) and diffuse intrinsic pontine glioma (DIPG)), CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma, or brain metastasis. In some embodiments, the individual may be a human who has a gene, genetic mutation, or polymorphism suspected or shown to be associated with the CNS tumor (e.g., mutation or polymorphism in NF1, NF2, SMARCB1, pVHL, TSC1, TSC2, p53, CHK2, MLH1, PMS2, PTCH, SUFU, and XRCC7) or has one or more extra copies of a gene associated with the CNS tumor.

In some embodiments of the methods described herein, the disease to be treated is a hematologic disease, and the pharmaceutical composition comprises a virus delivery complex or nanoparticle comprising one or more viruses that modifies the sequence and/or expression of one or more genes encoding proteins involved in hematologic disease development and/or progression. In some embodiments, the hematologic disease is a hemoglobinopathy, such as sickle-cell disease, thalassemia, or methemoglobinemia, an anemia, such as megaloblastic anemia, hemolytic anemia (e.g., hereditary spherocytosis, hereditary elliptocytosis, congenital dyserythropoietic anemia, glucose-6-phosphate dehydrogenase deficiency, pyruvate kinase deficiency, immune mediated hemolvtic anemia, autoimmune hemolytic anemia, warm antibody autoimmune hemolytic anemia, systemic lupus erythematosus, Evans' syndrome, cold autoimmune hemolytic anemia, cold agglutinin disease, paroxysmal cold hemoglobinuria, infectious mononucleosis, alloimmune hemolytic anemia, hemolytic disease of the newborn, or paroxysmal nocturnal hemoglobinuria), aplastic anemia (e.g., Fanconi anemia, Diamond-Blackfan anemia, or acquired pure red cell aplasia), myelodysplastic syndrome, myelofibrosis, neutropenia, agranulocytosis, Glanzmann's thrombasthenia, thrombocytopenia, a myeloproliferative disorder, such as polycethemia vera, erythrocytosis, leukocytosis, or thrombocytosis, or a coagulopathy, such as recurrent thrombosis, disseminated intravascular coagulation, hemophilia (e.g., hemophilia A, hemophilia B, or hemophilia C), Von Willebrand disease, protein S deficiency, antiphospholipid syndrome, or Wiskott-Aldrich syndrome. In some embodiments, the one or more genes encoding proteins involved in hematologic disease development and/or progression include, but are not limited to, HBA1, HBA2, HBB, PROC, ALAS2, ABCB7, SLC25A38, MTTP, FANCA, FANCB, FANCC, FANCD1 (BRCA2), FANCD2, FANCE, FANCF, FANCG, FANCI, FANCJ (BRIP1), FANCL, FANCM, FANCN (PALB2), FANCP (SLX4), FANCS (BRCA1), RAD51C, XPF, ANK1, SPTB, SPTA, SLC4A1, EPB42, and TPI1, including mutants thereof.

In some embodiments of the methods described herein, the disease to be treated is an organ-based disease, and the pharmaceutical composition comprises a virus delivery complex or nanoparticle comprising one or more viruses that modifies the sequence and/or expression of one or more genes encoding proteins involved in the organ-based disease development and/or progression. In some embodiments, the organ-based disease is a disease of the eye, liver, lung, kidney, heart, nervous system, muscle, or skin. In some embodiments, the disease is a cardiovascular disease, such as coronary heart disease, hypertension, atrial fibrillation, peripheral arterial disease, Marfan syndrome, long QT syndrome, or a congenital heart defect. In some embodiments, the disease is a digestive disease, such as irritable bowel syndrome, ulcerative colitis, Crohn's disease, celiac disease, peptic ulcer disease, gastroesophageal reflux disease, or chronic pancreatitis. In some embodiments, the disease is a urologic disease, such as chronic prostatitis, benign prostatic hyperplasia, or interstitial cystitis. In some embodiments, the disease is a musculoskeletal disease, such as osteoarthritis, osteoporosis, osteogenesis imperfecta, or Paget's disease of bone. In some embodiments, the disease is a skin disease, such as eczema, psoriasis, acne, rosacca, or dermatitis. In some embodiments, the disease is a dental or craniofacial disorder, such as periodontal disease or temporomandibular joint and muscle disorder (TMJD).

In some embodiments of the methods described herein, the disease to be treated is an ocular disease, and the pharmaceutical composition comprises a virus delivery complex or nanoparticle comprising one or more viruses that modifies the sequence and/or expression of one or more genes encoding proteins involved in ocular disease development and/or progression. In some embodiments, the ocular disease is age-related macular degeneration or the like, retinopathy of prematurity, polypoidal choroidal vasculopathy, diabetic retinopathy, diabetic macular edema, ischemic proliferative retinopathy, retinitis pigmentosa, cone dystrophy, proliferative vitreoretinopathy, retinal artery occlusion, retinal vein occlusion, keratitis, conjunctivitis, uveitis, Leber's disease, retinal detachment, retinal pigment epithelial detachment, neovascular glaucoma, corneal neovascularization, retinochoroidal neovascularization, or inherited retinal disease (such as RPE65-mediated IRD, choroideremia, rhodopsin-linked autosomal dominant retinitis pigmentosa (RHO-adRP), Leber hereditary optic neuropathy (LHON), or Leber congenital amaurosis). In some embodiments, the one or more genes encoding proteins involved in occular disease development and/or progression include, but are not limited to, Rho, PDE6β, ABCA4, RPE65, LRAT, RDS/Peripherin, MERTK, CNGA1, RPGR, IMPDH1, ChR2, GUCY2D, RDS/Peripherin, AIPL1, ABCA4, RPGRIP1, IMPDH1, AIPL1, GUCY2D, LRAT, MERTK, RPGRIP1, RPE65, CEP290, ABCA4, DFNB31, MYO7A, USH1C, CDH23, PCDH15, USH1G, CLRN 1, GNAT2, CNGA3, CNGB3, Rs1, OA1, MT-ND4, (OCA1), tyrosinase, p21 WAF-1/OCip1, REP-1, PDGF, Endostatin, Angiostatin, VEGF inhibitor, Opsin, OPN1LW, arylsulfatase B, and β-glucuronidase, including mutants thereof.

In some embodiments of the methods described herein, the disease to be treated is a liver disease, and the pharmaceutical composition comprises a virus delivery complex or nanoparticle comprising one or more viruses that modifies the sequence and/or expression of one or more genes encoding proteins involved in liver disease development and/or progression. In some embodiments, the liver disease is hepatitis, fatty liver disease (alcoholic and nonalcoholic), hemochromatosis, Wilson's disease, progressive familial intrahepatic cholestasis type 3, hereditary fructose intolerance, glycogen storage disease type IV, tyrosinemia type I, argininosuccinate lyase deficiency, citrin deficiency (CTLN2, NICCD), cholesteryl ester storage disease, cystic fibrosis, Alström syndrome, congenital hepatic fibrosis, alpha 1-antitrypsin deficiency, glycogen storage disease type II, transthyretin-related hereditary amyloidosis. Gilbert's syndrome, cirrhosis, primary biliary cirrhosis, primary sclerosing cholangitis, or hemophilia (such as hemophilia A or hemophilia B). In some embodiments, the one or more genes encoding proteins involved in liver disease development and/or progression include, but are not limited to, ATP7B, ABCB4, ALDOB, GBE1, FAH, ASL, SLC25A13, LIPA, CFTR, ALMS1, HFE, HFE2, HFDE2B, HFE3, SLC11A3/SLC40A 1, ceruloplasmin, transferrin, A1AT, BCS1L, B3GAT1, B3GAT2, B3GAT3, UGT1A1, UGT1A3, UGT1A4, UGT1A5, UGT1A6, UGT1A7, UGT1A8, UGT1A9, UGTIA10, UGT2A1, UGT2A2, UGT2A3, UGT2B4, UGT2B7, GT2B10, UGT2B11, UGT2B15, UGT2B17, UGT2B28, Factor IX, and Factor VIII, including mutants thereof.

In some embodiments of the methods described herein, the disease to be treated is a lung disease, and the pharmaceutical composition comprises a virus delivery complex or nanoparticle comprising one or more viruses that modifies the sequence and/or expression of one or more genes encoding proteins involved in lung disease development and/or progression. In some embodiments, the lung disease is chronic obstructive pulmonary disease (COPD), asthma, cystic fibrosis, primary ciliary dyskinesia, pulmonary fibrosis, Birt Hogg Dube syndrome, tuberous sclerosis, Kartagener syndrome, α₁-antitrypsin deficiency, pulmonary capillary hemangiomatosis (PCH), or hereditary heamorrhagic telangiectasia. In some embodiments, the one or more genes encoding proteins involved in lung disease development and/or progression include, but are not limited to, EIF2AK4, IREB2, HHIP, FAM13A, IL1RL1, TSLP, IL33, IL25, CFTR, DNAI1, DNAH5, TXNDC3, DNAH11, DNAI2, KTU, RSPH4A, RSPH9, LRRC50, TERC, TERT, SFTPC, SFTPA2, FLCN, TSC1, TSC2, A1AT, ENG, ACVRL1, and MADH4, including mutants thereof.

In some embodiments of the methods described herein, the disease to be treated is a kidney disease, and the pharmaceutical composition comprises a virus delivery complex or nanoparticle comprising one or more viruses that modifies the sequence and/or expression of one or more genes encoding proteins involved in kidney disease development and/or progression. In some embodiments, the kidney disease is cystic kidney disease (e.g., autosomal dominant polycystic kidney disease, autosomal recessive polycystic kidney disease, nephronophthisis, or medullary sponge kidney), Alport's syndrome, Bartter's syndrome, congenital nephrotic syndrome, nail-patella syndrome, primary immune glomerulonephritis, reflux nephropathy, or haemolytic uraemic syndrome. In some embodiments, the one or more genes encoding proteins involved in kidney disease development and/or progression include, but are not limited to, PKD1, PKD2, PKD3, fibrocystin, NPHP1, NPHP2, NPHP3, NPHP4, NPHP5, NPHP6, NPHP7, NPHP8, NPHP9, NPHP11, NPHP11, NPHPL1, GDNF, COL4A5, COL4A3, COL4A4, SLC12A2 (NKCC2), ROMK/KCNJ1, CLCNKB, BSND, CASR, SLC12A3 (NCCT), and ADAMTS13, including mutants thereof.

In some embodiments of the methods described herein, the disease to be treated is a muscle disease, and the pharmaceutical composition comprises a virus delivery complex or nanoparticle comprising one or more viruses that modifies the sequence and/or expression of one or more genes encoding proteins involved in muscle disease development and/or progression. In some embodiments, the muscle disease is myopathy (e.g., mitochondrial myopathy), muscular dystrophy (e.g., Duchenne, Becker, Emery-Dreifuss, facioscapulohumeral, myotonic, congenital, distal, limb-girdle, and oculopharyngeal), cerebral palsy, fibrodysplasia ossificans progressiva, dermatomyositis, compartment syndrome, myasthenia gravis, amyotrophic lateral sclerosis, rhabdomyolysis, polymyositis, fibromyalgia, myotonia, myofascial pain syndrome. In some embodiments, the one or more genes encoding proteins involved in muscle disease development and/or progression include, but are not limited to, DMD, LAMA2, collagen VI (COL6A 1, COL6A2, or COL6A3), POMT1, POMT2, FKTN, FKRP, LARGE1, POMGNT1, ISPD, SEPN1, LMNA, DYSF, EMD, DUX4, DMPK, ZNF9, PABPN1, CAV3, CAPN3, SGCA, SGCB, SGCG, SGCD, TTN, ANO5, DNAJB6, HNRNPDL, MYOT, TCAP, TNPO3, TRAPPCI 1, and TRIM32, including mutants thereof.

In some embodiments of the methods described herein, the disease to be treated is a nervous system disease (such as a central nervous system disease), and the pharmaceutical composition comprises a virus delivery complex or nanoparticle comprising one or more viruses that modifies the sequence and/or expression of one or more genes encoding proteins involved in nervous system disease development and/or progression. In some embodiments, the nervous system disease is adrenoleukodystrophy, Angelman syndrome, ataxia telangiectasia, Charcot-Marie-Tooth syndrome, Cockayne syndrome, essential tremor, fragile X syndrome, Friedreich's ataxia, Gaucher disease, Lesch-Nyhan syndrome, maple syrup urine disease, Menkes syndrome, narcolepsy, neurofibromatosis, Niemann-Pick disease, phenylketonuria, Prader-Willi syndrome, Refsum disease, Rett syndrome, spinal muscular atrophy, spinocerebellar ataxia, Tangier disease, Tav-Sachs disease, tuberous sclerosis, Von Hippel-Lindau syndrome, Williams syndrome, Wilson's disease, Zellweger syndrome, attention deficit/hyperactivity disorder (ADHD), autism, bipolar disorder, depression, epilepsy, migraine, multiple sclerosis, myelopathy, Alzheimer's, Huntington's, Parkinson's, Tourette's, CLN2 disease (such as CLN2 disease caused by TPP1 deficiency), or mucopolysaccharidosis (such as mucopolysaccharidosis type I (MPS I) or mucopolysaccharidosis type II (MPS II)). In some embodiments, the one or more genes encoding proteins involved in nervous system disease development and/or progression include, but are not limited to, ALD, PS1 (AD3), PS2 (AD4), SOD1, UBE3A, ATM, PMP22, MPZ, LITAF, EGR2, MFN2, KIF1B, RAB7A, LMNA, TRPV4, BSCL2, GARS, NEFL, HSPB1, GDAP1, HSPB8, MTMR2, SBF2, SH3TC2, NDRG1, PRX, FGD4, FIG4, DNM2, YARS, GJB1, PRPS1, CSA, CSB, Cx26, EPM2A, ETM1(FETI), ETM2, FMR1, frataxin, GBA, HTT, HPRT1, BCKDH, ATP7A, ATP7B, HLA-DQB1, HLA-DQA1, HLA-DRB1, NF1, NF2, SMPD1, NPC1, NPC2, LRRK2, PARK7, PINK1, PRKN, SNCA, UCHL1, PAH, PHYH, PEX7, MeCP2, SMN1, ATXN genes (e.g., ATXN1, ATXN2, etc), ABC1, HEXA, TSC1, TSC2, VHL, CLIP2, ELN, GTF2I, GTF2IRD1, LIMK1, PXR1, TPP1, IDUA, and IDS, including mutants thereof.

In some embodiments of the methods described herein, the disease to be treated is cancer, wherein the cancer is a hematological malignancy, and the pharmaceutical composition comprises a virus delivery complex or nanoparticle comprising one or more viruses that modifies the sequence and/or expression of one or more genes encoding proteins involved in hematological malignancy development and/or progression. In some embodiments, the one or more genes encoding proteins involved in hematological malignancy development and/or progression include, but are not limited to, GLI1, CTNNB1, eIF5A, mutant DDX3X, Hexokinase II, histone methyltransferase EZH2, ARK5, ALK, MUC 1, HMGA2, IRF1, RPN13, HDAC11, Rad51, Spry2, mir-146a, mir-146b, survivin, MDM2, MCL1, CMYC, XBP1 (spliced and unspliced), SLAMF7, CS1, Erbb4, Cxcr4 (waldenstroms macroglobulinemia), Myc, Bcl2, Prdx1 and Prdx2 (burkitts lymphoma), Bcl6, Idh1, Idh2, Smad, Ccnd2, Cyclin d1-2, B7-h1 (pdl-1), and Pyk2, including mutants thereof.

In some embodiments of the methods described herein, the disease to be treated is a viral infectious disease, and the pharmaceutical composition comprises a virus delivery complex or nanoparticle comprising one or more viruses that modifies the sequence and/or expression of one or more genes encoding proteins involved in the viral infectious disease development and/or progression. In some embodiments, the viral infectious disease is characterized by infection with hepatitis virus, human immunodeficiency virus (HIV), picomavirus, poliovirus, enterovirus, human Coxsackie virus, influenza virus, rhinovirus, echovirus, rubella virus, encephalitis virus, rabies virus, herpes virus, papillomavirus, polyoma virus, RSV, adenovirus, yellow fever virus, dengue virus, parainfluenza virus, hemorrhagic virus, pox virus, varicella zoster virus, parainfluenza virus, reovirus, orbivirus, rotavirus, parvovirus, African swine fever virus, measles, mumps or Norwalk virus. In some embodiments, the viral infectious disease is characterized by infection with an oncogenic virus including, but not limited to, CMV, EBV. HBV, KSHV, HPV, MCV, HTLV-1, HIV-1, and HCV In some embodiments, the one or more genes encoding proteins involved in the viral infectious disease development and/or progression include, but are not limited to, genes encoding RSV nucleocapsid, Pre-gen/Pre-C, Pre-S1, Pre-S2/S,X, HBV conserved sequences, HIV Gag polyprotein (p55), HIV Pol polyprotein, HIV Gag-Pol precursor (p160), HIV matrix protein (MA, p17), HIV capsid protein (CA, p24), HIV spacer peptide 1 (SP1, p2), HIV nucleocapsid protein (NC, p9), HIV spacer peptide 2 (SP2, p1), HIV P6 protein, HIV reverse transcriptase (RT, p50), HIV RNase H (p15), HIV integrase (IN, p31), HIV protease (PR, p10), HIV Env (gp160), gp120, gp41, HIV transactivator (Tat), HIV regulator of expression of virion proteins (Rev), HIV lentivirus protein R (Vpr), HIV Vif, HIV negative factor (Nef), HIV virus protein U (Vpu), human CCR5, miR-122, EBOV polymerase L, VP24, VP40, GP/sGP, VP30, VP35, NPC1, and TIM-1, including mutants thereof.

In some embodiments of the methods described herein, the disease or condition to be treated is an autoimmune or inflammatory disease or condition, and the pharmaceutical composition comprises a virus delivery complex or nanoparticle comprising one or more viruses that modifies the sequence and/or expression of one or more genes encoding proteins involved in the autoimmune or inflammatory disease or condition development and/or progression. In some embodiments, the autoimmune or inflammatory disease or condition is acne, allergies, anaphylaxis, asthma, celiac disease, diverticulitis, glomerulonephritis, inflammatory bowel disease, interstitial cystitis, lupus, otitis, pelvic inflammatory disease, rheumatoid arthritis, sarcoidosis, or vasculitis. In some embodiments, the one or more genes encoding proteins involved in the autoimmune or inflammatory disease or condition development and/or progression include, but are not limited to, genes encoding molecules of the complement system (CD46, CD59, CFB, CFD, CFH, CFHR1, CFHR2, CFHR3, CFHR4, CFHR5, CFI, CFP, CR1, CR1L, CR2, C1QA, C1QB, C1QC, C1R, C1S, C2, C3, C3AR1, C4A, C4B, C5, C5AR1, C6, C7, C8A, C8B, C8G, C9, ITGAM, ITGAX, and ITGB2), including mutants thereof.

In some embodiments of the methods described herein, the disease or condition to be treated is a lysosomal storage disease, and the pharmaceutical composition comprises a virus delivery complex or nanoparticle comprising one or more viruses that modifies the sequence and/or expression of one or more genes encoding proteins involved in the lysosomal storage disease development and/or progression. In some embodiments, the lysosomal storage disease is Sphingolipidoses (e.g., Farber disease, Krabbe disease (infantile onset, late onset), galactosialidosis, gangliosidoses (e.g., Fabry disease, Schindler disease, GM1 gangliosidosis (Infantile, Juvenile, Adult/Chronic), GM2 gangliosidosis (e.g., Sandhoff disease (Infantile, Juvenile, Adult onset), Tay-Sachs)), Gaucher Disease (Type 1, Type II, Type III), Lysosomal acid lipase deficiency (Early onset, Late onset), Niemann-Pick disease (Type A, Type B), Sulfatidosis (e.g., Metachromatic Leukodystrophy (MLD), Multiple sulfatase deficiency)), Mucopolysaccharidoses (e.g., MPS I Hurler Syndrome, MPS I S Scheie Syndrome, MPS I H-S Hurler-Scheic Syndrome, Type II (Hunter syndrome), Type III (Sanfilippo syndrome), Type IV (Morquio), Type VI (Maroteaux-Lamy syndrome), Type VII (Sly Syndrome), Type IX (Hyaluronidase deficiency)), Mucolipidosis (e.g., Type I (Sialidosis), Type II (I-cell disease), Type III (Pseudo-Hurler Polydystrophy/Phosphotransferase deficiency), Type IV (Mucolipidin 1 deficiency)), Lipidoses (e.g., Niemann-Pick disease (Type C, Type D), Neuronal Ceroid Lipofuscinoses, Wolman disease). Alpha-mannosidosis, Beta-mannosidosis, Aspartylglucosaminuria, Fucosidosis, Lysosomal Transport Diseases (e.g., Cystinosis, Pycnodysostosis, Salla disease/Sialic Acid Storage Disease, Infantile Free Sialic Acid Storage Disease (ISSD)), Cholesteryl ester storage disease. In some embodiments, the one or more genes encoding proteins involved in the lysosomal storage disease development and/or progression include, but are not limited to, genes encoding ceramidase, Alpha-galactosidase (A, B), Beta-galactosidase, Hexosaminidase A, Sphingomyelinase, Lysosomal acid lipase, Saposin B, sulfatase. Hyaluronidase, Phosphotransferase, Mucolipidin 1, aspartylglucosaminidase, alpha-D-mannosidase, beta-mannosidase, alpha-L-fucosidase, cystinosin, cathepsin K, sialin, SLC17A5, acid alpha-glucosidase, LAMP2, including mutants thereof.

In some embodiments of the methods described herein, the disease or condition to be treated is a glycogen storage disease, and the pharmaceutical composition comprises a virus delivery complex or nanoparticle comprising one or more viruses that modifies the sequence and/or expression of one or more genes encoding proteins involved in the glycogen storage disease development and/or progression. In some embodiments, the glycogen storage disease is von Gierke's disease, Pompe's disease, Cori's disease or Forbes' disease, Andersen disease, McArdle disease, Hers' disease, Tarui's disease, Fanconi-Bickel syndrome, or Red cell aldolase deficiency. In some embodiments, the one or more genes encoding proteins involved in the glycogen storage disease development and/or progression include, but are not limited to, genes encoding glycogen synthase, glucose-6-phosphatase, acid alpha-glucosidase, glycogen debranching enzyme, glycogen branching enzyme, muscle glycogen phosphorylase, liver glycogen phosphorylase, muscle phosphofructokinase, Phosphorylase kinase, glucose transporter, GLUT2, Aldolase A, and β-enolase, including mutants thereof.

In some embodiments of the methods described herein, the disease or condition to be treated is an immunodeficiency disease, and the pharmaceutical composition comprises a virus delivery complex or nanoparticle comprising one or more viruses that modifies the sequence and/or expression of one or more genes encoding proteins involved in the glycogen storage disease development and/or progression. In some embodiments, the glycogen storage disease is von Gierke's disease, Pompe's disease, Cori's disease or Forbes' disease, Andersen disease, McArdle disease, Hers' disease, Tarui's disease, Fanconi-Bickel syndrome, or Red cell aldolase deficiency. In some embodiments, the one or more genes encoding proteins involved in the glycogen storage disease development and/or progression include, but are not limited to, genes encoding glycogen synthase, glucose-6-phosphatase, acid alpha-glucosidase, glycogen debranching enzyme, glycogen branching enzyme, muscle glycogen phosphorylase, liver glycogen phosphorylase, muscle phosphofructokinase, Phosphorylase kinase, glucose transporter, GLUT2, Aldolase A, and β-enolase, including mutants thereof.

In some embodiments of the methods described herein, the condition to be treated is characterized by abnormal cholesterol levels (such as abnormally high LDL levels, e.g., LDL above about 100 mg/dL, and/or abnormally low HDL levels, e.g., HDL below about 40-50 mg/dL), including, e.g., familial hypercholesterolemia (such as homozygous familial hypercholesterolemia (HoFH)), and the pharmaceutical composition comprises a virus delivery complex or nanoparticle comprising one or more viruses that modifies the sequence and/or expression of one or more genes encoding proteins involved in cholesterol transport and/or metabolism. In some embodiments, the one or more genes encoding proteins involved in cholesterol transport and/or metabolism include, but are not limited to, low-density lipoprotein (LDL) receptor (LDLR), apolipoprotein B (ApoB), low-density lipoprotein receptor adapter protein 1 (LDLRAP1), and PCSK9, including mutants thereof.

In some embodiments, a virus delivery complex or nanoparticle as described herein is used to activate LDLR expression. In some embodiments, a virus delivery complex or nanoparticle as described herein is used to correct a mutation in a gene encoding LDLR. In some embodiments, a virus delivery complex or nanoparticle as described herein is used to introduce a gene encoding LDLR.

In some embodiments, a virus delivery complex or nanoparticle as described herein is used to activate ApoB expression. In some embodiments, a virus delivery complex or nanoparticle as described herein is used to correct a mutation in a gene encoding ApoB. In some embodiments, a virus delivery complex or nanoparticle as described herein is used to introduce a gene encoding ApoB.

In some embodiments, a virus delivery complex or nanoparticle as described herein is used to activate LDLRAP1 expression. In some embodiments, a virus delivery complex or nanoparticle as described herein is used to correct a mutation in a gene encoding LDLRAP1. In some embodiments, a virus delivery complex or nanoparticle as described herein is used to introduce a gene encoding LDLRAP1.

In some embodiments, a virus delivery complex or nanoparticle as described herein is used to repress PCSK9 expression, such as by gene knockout.

In some embodiments of the methods described herein, the disease to be treated is a genetic disease, such as a hereditary disease, and the pharmaceutical composition comprises a virus delivery complex or nanoparticle comprising one or more viruses that modifies the sequence and/or expression of one or more genes encoding proteins involved in the genetic disease development and/or progression. In some embodiments, the virus corrects a mutation in one or more genes encoding proteins involved in the genetic disease development and/or progression. In some embodiments, the genetic disease includes, but is not limited to, 22q11.2 deletion syndrome, achondroplasia, Alpha-1 Antitrypsin Deficiency, Angelman syndrome, Autosomal dominant polycystic kidney disease, breast cancer, Canavan disease, Charcot-Marie-Tooth disease, colon cancer, Color blindness, Cystic fibrosis, Duchenne muscular dystrophy. Factor V Leiden thrombophilia, Familial Mediterranean Fever, Fragile X syndrome, Gaucher disease, Haemochromatosis, Haemophilia, Huntington's disease, Marfan syndrome, Myotonic dystrophy, Osteogenesis imperfecta, Parkinson's disease, Phenylketonuria, Polycystic kidney disease, porphyria, Prader-Willi syndrome, progeria, SCID, Sickle-cell disease, Spinal muscular atrophy, Tay-Sachs disease, thalassemia, Trimethylamine, and Wilson's disease. In some embodiments, the genes involved in the genetic disease development and/or progression include, but are not limited to, AAT, ADA, ALAD, ALAS2, APC, ASPM, ATP7B, BDNF, BRCA1, BRCA2, CFTR, COL1A1, COL1A2, COMT, CNBP, CPOX, CREBBP, CRH, CRTAP, CXCR4, DHFR, DMD, DMPK, F5, FBN1, FECHFGFR3, FGR3, FIX, FVIII, FMO3, FMR1, GARS, GBA, HBB, HEXA, HFE, HMBS, HTT, IL2RG, KRT14, KRT5, LMNA, LRRK2, MEFV, MLH1, MSH2, MSH6, PAH, PARK2, PARK3, PARK7, PGL2, PHF8, PINK1, PKD1, PKD2, PMS1, PMS2, PPOX, RHO, SDHB, SDHC, SDHD, SMNI, SNCA, SRY, TSC1, TSC2, UCHL1, UROD, UROS, MEFV, APP, GAST, INS, LCK, LEP, LIF, MCM6, MYH7, MYOD1, NPPB, OSM, PKC, PIP, SLC18A2, TBX1, Transthyretin, MDS1-EVI1, PRDM16, SETBP1, β-Globin, and LPL, including mutants thereof.

In some embodiments of the methods described herein, the disease to be treated is an aging or degenerative disease, and the pharmaceutical composition comprises a virus delivery complex or nanoparticle comprising one or more viruses that modifies the sequence and/or expression of one or more genes encoding proteins involved in the aging or degenerative disease development and/or progression. In some embodiments, the one or more genes encoding proteins involved in the aging or degenerative disease development and/or progression include, but are not limited to, keratin K6A, keratin K6B, keratin 16, keratin 17, p53, β-2 adrenergic receptors (ADRB2), TRPV1, VEGF, VEGFR, HIF-1, and caspase-2, including mutants thereof.

In some embodiments of the methods described herein, the disease to be treated is a fibrotic or inflammatory disease, and the pharmaceutical composition comprises a virus delivery complex or nanoparticle comprising one or more viruses that modifies the sequence and/or expression of one or more genes encoding proteins involved in the fibrotic or inflammatory disease development and/or progression. In some embodiments, the one or more genes encoding proteins involved in the fibrotic or inflammatory disease development and/or progression are selected from the group consisting of SPARC, CTGF, TGFβ1, TGFβ receptors 1, TGFβ receptors 2, TGFβ receptors 3, VEGF, Angiotensin II, TIMP, HSP47, thrombospondin, CCN1, LOXL2, MMP2, MMP9, CCL2, Adenosine receptor A2A, Adenosine receptor A2B, Adenylyl cyclase, Smad 3, Smad 4, Smad 7, SOX9, arrestin, PDCD4, PAI-1, NF-κB, and PARP-1, including mutants thereof.

In some embodiments of the methods described herein, the pharmaceutical composition comprises a virus delivery complex or nanoparticle comprising one or more viruses that modulates the expression of one or more miRNAs involved in a disease or condition. In some embodiments, the disease or condition includes, but is not limited to, hepatitis B, hepatitis C, polycystic liver and kidney disease, cancer, cardiovascular disease, cardiac failure, cardiac hypertrophy, neurodevelopmental disease, fragile X syndrome, Rett syndrome, Down syndrome, Alzheimer's disease, Huntington's disease, schizophrenia, inflammatory disease, rheumatoid arthritis, systemic lupus erythematosus, psoriasis, and skeletal muscle disease. In some embodiments, the one or more miRNAs include, but are not limited to, has-mir-126*, Has-miR-191, has-mir-205, has-mir-21, hsa-let-7a-2, let-7 family, let-7c, let-7f-1, miR-1, miR-100, miR-103, miR-103-1, miR-106b-25, miR-107, miR-10b, miR-112, miR-122, miR-125b, miR-125b-2, miR125b1, miR-126, miR-128a, mIR-132, miR-133, miR-133b, miR135, miR-140, miR-141, miR-142-3p, miR143, miR-143, miR145, miR-145, miR-146, miR-146b, miR150, miR-155, miR-15a, miR-15b, miR16, miR-16, miR-17-19 family, miR-173p, miR17-5p, miR-17-5p, miR-17-92, miR-181a, miR-181b, miR-184, miR-185, miR-189, miR-18a, miR-191, miR-192, miR-193a, miR-193b, miR-194, miR-195, miR-196a, miR-198, miR-199, miR-199a, miR-19a, miR-19b-1, miR200a, miR-200a miR-200b, miR200c, miR-200c, miR-203, miR-205, miR-208, miR-20a, miR-21, miR-214, miR-221, miR-222, miR-223, miR-224, miR-23, miR-23a, miR-23b, miR-24, miR-26a, miR-26b, miR-27b, miR-29, miR-298, miR-299-3p, miR-29c, miR-30a-5p, miR-30c, miR-30d, miR-30e-5p, miR31, miR-34, miR342, miR-381, miR-382, miR-383, miR-409-3p, miR-45, miR-61, miR-78, miR-802, miR-9, miR-92a-1, miR-99a, miR-let7, miR-let7a, and miR-let7g.

In some embodiments of the methods described herein, the pharmaceutical composition is administered to the individual by any of intravenous, intratumoral, intraarterial, topical, intraocular, ophthalmic, intraportal, intracranial, intracerebral, intracerebroventricular, intrathecal, intravesicular, intradermal, subcutaneous, intramuscular, intranasal, intratracheal, pulmonary, intracavity, or oral administration.

In some embodiments of the methods described herein, the individual is a mammal. In some embodiments, the individual is human.

### Methods of Cell Delivery

In some embodiments, there is provided a method of delivering one or more viruses into a cell comprising contacting the cell with a virus delivery complex or nanoparticle as described herein, wherein the complex or nanoparticle comprises the one or more viruses. In some embodiments, the complex or nanoparticle comprises a CPP comprising the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the contacting of the cell with the complex or nanoparticle is carried out in vivo. In some embodiments, the contacting of the cell with the complex or nanoparticle is carried out ex vivo. In some embodiments, the contacting of the cell with the complex or nanoparticle is carried out in vitro. In some embodiments, the cell is an immortalized cell, such as a cell from a cell line. In some embodiments, the cell is a primary cell, such as a cell from an individual. In some embodiments, the cell is an immune cell, such as a granulocyte, a mast cell, a monocyte, a dendritic cell, a B cell, a T cell, or a natural killer cell. In some embodiments, the cell is a peripheral blood-derived T cell, a central memory T cell, a cord blood-derived T cell, or a hematopoietic stem cell or other precursor cell. In some embodiments, the T cell is an immortalized T cell, such as a T cell from a T cell line. In some embodiments, the T cell is a primary T cell, such as a T cell of an individual. In some embodiments, the cell is a T cell, and the contacting is carried out after activating the T cell. In some embodiments, the cell is a T cell, and the contacting is carried out at least 12 hours (such as at least about any of 12 hours, I day, 2 days, 3 days, 4 days, 5 days, 6 days, or more) after activating the T cell. In some embodiments, the T cell is activated using an anti-CD3/CD28 reagent (such as microbeads). In some embodiments, the cell is a fibroblast. In some embodiments, the fibroblast is a primary fibroblast, such as a fibroblast of an individual. In some embodiments, the cell is a muscle cell. In some embodiments, the cell is a cardiac cell. In some embodiments, the cell is a hepatocyte. In some embodiments, the hepatocyte is a primary hepatocyte, such as a hepatocyte of an individual. In some embodiments, the cell is a human lung progenitor cell (LPC). In some embodiments, the cell is a neuronal cell. In some embodiments, the virus include recombinant AAV, adenovirus, lentivirus, retrovirus, HSV, poxvirus, EBV, vaccinia virus, and hCMV. In some embodiments, the virus is useful for the treatment of a disease, such as any of the diseases to be treated described herein (e.g., cancer, diabetes, autoimmune diseases, inflammatory diseases, fibrotic diseases, viral infectious diseases, hereditary diseases, ocular diseases, and aging and degenerative diseases). In some embodiments, the complex or nanoparticle further comprises one or more additional viruses. In some embodiments, the additional virus is useful for the treatment of the disease.

Thus, in some embodiments, there is provided a method of delivering one or more viruses into a cell comprising contacting the cell with a virus delivery complex or nanoparticle as described herein, wherein the virus delivery complex or nanoparticle comprises the one or more viruses and a CPP comprising the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the virus is a recombinant virus, including recombinant AAV, adenovirus, lentivirus, retrovirus, HSV, poxvirus, EBV, vaccinia virus, and hCMV. In some embodiments, the recombinant virus comprises a transgene for insertion into a cell genome. In some embodiments, the transgene is a therapeutic transgene. In some embodiments, the transgene encodes a protein, such as a therapeutic protein. In some embodiments, the transgene encodes an inhibitory RNA (RNAi), such as an RNAi targeting an endogenous gene, e.g., a disease-associated endogenous gene. In some embodiments, the transgene encodes a CAR. In some embodiments, the complex or nanoparticle comprises one or more viruses comprising a first transgene encoding an RNAi and a second transgene encoding a protein. In some embodiments, the RNAi is a therapeutic RNAi targeting an endogenous gene involved in a disease or condition, and the protein is a therapeutic protein useful for treating the disease or condition. In some embodiments, the therapeutic RNAi targets a disease-associated form of the endogenous gene (e.g., a gene encoding a mutant protein, or a gene resulting in abnormal expression of a protein), and the second transgene is a therapeutic form of the endogenous gene (e.g., the second transgene encodes a wild-type or functional form of the mutant protein, or the second transgene results in normal expression of the protein). In some embodiments, the complex or nanoparticle comprises a first virus comprising the first transgene and a second virus comprising the second transgene. In some embodiments, the complex or nanoparticle comprises a single virus comprising the first transgene and the second transgene. In some embodiments, the contacting of the cell with the complex or nanoparticle is carried out in vivo. In some embodiments, the contacting of the cell with the complex or nanoparticle is carried out ex vivo. In some embodiments, the contacting of the cell with the complex or nanoparticle is carried out in vitro. In some embodiments, the cell is an immortalized cell, such as a cell from a cell line. In some embodiments, the cell is a primary cell, such as a cell from an individual. In some embodiments, the cell is an immune cell, such as a granulocyte, a mast cell, a monocyte, a dendritic cell, a B cell, a T cell, or a natural killer cell. In some embodiments, the T cell is an immortalized T cell, such as a T cell from a T cell line. In some embodiments, the T cell is a primary T cell, such as a T cell of an individual. In some embodiments, the cell is a fibroblast. In some embodiments, the fibroblast is a primary fibroblast, such as a fibroblast of an individual. In some embodiments, the cell is a muscle cell. In some embodiments, the cell is a cardiac cell. In some embodiments, the cell is a hepatocyte. In some embodiments, the hepatocyte is a primary hepatocyte, such as a hepatocyte of an individual. In some embodiments, the cell is a human lung progenitor cell (LPC). In some embodiments, the cell is a neuronal cell. In some embodiments, the virus is useful for the treatment of a disease, such as any of the diseases to be treated described herein (e.g., cancer, diabetes, autoimmune diseases, inflammatory diseases, fibrotic diseases, viral infectious diseases, hereditary diseases, ocular diseases, and aging and degenerative diseases). In some embodiments, the virus is useful for modulating a protein involved in a disease, such as any of the diseases to be treated described herein (e.g., cancer, diabetes, autoimmune diseases, inflammatory diseases, fibrotic diseases, viral infectious diseases, hereditary diseases, ocular diseases, and aging and degenerative diseases). In some embodiments, the cell-penetrating peptide is an ADGN-100 peptide or a VEPEP-3 peptide.

In some embodiments, there is provided a method of delivering one or more viruses into a T cell comprising contacting the cell with a virus delivery complex or nanoparticle as described herein, wherein the complex or nanoparticle comprises the one or more viruses and a CPP selected from the group consisting of PEP-1 peptides, PEP-2 peptides, PEP-3 peptides, VEPEP-3 peptides, VEPEP-6 peptides, VEPEP-9 peptides, and ADGN-100 peptides. In some embodiments, the contacting of the T cell with the complex or nanoparticle is carried out in vivo. In some embodiments, the contacting of the T cell with the complex or nanoparticle is carried out ex vivo. In some embodiments, the contacting of the T cell with the complex or nanoparticle is carried out in vitro. In some embodiments, the T cell is an immortalized T cell, such as a T cell from a T cell line. In some embodiments, the T cell is a primary T cell, such as a T cell of an individual. In some embodiments, the one or more viruses include recombinant AAV, adenovirus, lentivirus, retrovirus, HSV, poxvirus, EBV, vaccinia virus, and hCMV. In some embodiments, the virus is useful for the treatment of a disease, such as any of the diseases to be treated described herein. In some embodiments, the complex or nanoparticle further comprises one or more additional viruses. In some embodiments, the additional virus is useful for the treatment of the disease.

In some embodiments, there is provided a method of delivering one or more viruses into a fibroblast comprising contacting the fibroblast with a virus delivery complex or nanoparticle as described herein, wherein the complex or nanoparticle comprises the one or more viruses and a CPP selected from the group consisting of PEP-1 peptides, PEP-2 peptides, PEP-3 peptides, VEPEP-3 peptides. VEPEP-6 peptides, VEPEP-9 peptides, and ADGN-100 peptides. In some embodiments, the contacting of the fibroblast with the complex or nanoparticle is carried out in vivo. In some embodiments, the contacting of the fibroblast with the complex or nanoparticle is carried out ex vivo. In some embodiments, the contacting of the fibroblast with the complex or nanoparticle is carried out in vitro. In some embodiments, the fibroblast is an immortalized fibroblast, such as a fibroblast from a fibroblast line. In some embodiments, the fibroblast is a primary fibroblast, such as a fibroblast of an individual. In some embodiments, the one or more viruses include recombinant AAV, adenovirus, lentivirus, retrovirus, HSV, poxvirus. EBV, vaccinia virus, and hCMV. In some embodiments, the virus is useful for the treatment of a disease, such as any of the diseases to be treated described herein. In some embodiments, the complex or nanoparticle further comprises one or more additional viruses. In some embodiments, the additional virus is useful for the treatment of the disease.

In some embodiments, there is provided a method of delivering one or more viruses into a hepatocyte comprising contacting the hepatocyte with a virus delivery complex or nanoparticle as described herein, wherein the complex or nanoparticle comprises the one or more viruses and a CPP selected from the group consisting of PEP-1 peptides, PEP-2 peptides, PEP-3 peptides, VEPEP-3 peptides, VEPEP-6 peptides, VEPEP-9 peptides, and ADGN-100 peptides. In some embodiments, the contacting of the hepatocyte with the complex or nanoparticle is carried out in vivo. In some embodiments, the contacting of the hepatocyte with the complex or nanoparticle is carried out ex vivo. In some embodiments, the contacting of the hepatocyte with the complex or nanoparticle is carried out in vitro. In some embodiments, the hepatocyte is an immortalized hepatocyte, such as a hepatocyte from a hepatocyte line. In some embodiments, the hepatocyte is a primary hepatocyte, such as a hepatocyte of an individual. In some embodiments, the one or more viruses include recombinant AAV, adenovirus, lentivirus, retrovirus, HSV, poxvirus, EBV, vaccinia virus, and hCMV. In some embodiments, the virus is useful for the treatment of a disease, such as any of the diseases to be treated described herein. In some embodiments, the complex or nanoparticle further comprises one or more additional viruses. In some embodiments, the additional virus is useful for the treatment of the disease.

In some embodiments, there is provided a method of delivering one or more viruses into a cell in an individual comprising administering to the individual a composition comprising a virus delivery complex or nanoparticle as described herein, wherein the complex or nanoparticle comprises the one or more viruses and a CPP selected from the group consisting of PEP-1 peptides, PEP-2 peptides, PEP-3 peptides, VEPEP-3 peptides, VEPEP-6 peptides, VEPEP-9 peptides, and ADGN-100 peptides. In some embodiments, the composition is administered to the individual via an intravenous, intraarterial, intraperitoneal, intravesicular, subcutaneous, intrathecal, intracranial, intracerebral, intracerebroventricular, intrapulmonary, intramuscular, intratracheal, intraocular, ophthalmic, intraportal, transdermal, intradermal, oral, sublingual, topical, or inhalation route. In some embodiments, the composition is administered to the individual via an intravenous route. In some embodiments, the composition is administered to the individual via a subcutaneous route. In some embodiments, the cell is present in an organ or tissue including lung, liver, brain, kidney, heart, spleen, blood, pancreas, muscle, bone marrow, and intestine. In some embodiments, the cell is present in the lung, liver, kidney, or spleen of the individual. In some embodiments, the one or more viruses include recombinant AAV, adenovirus, lentivirus, retrovirus. HSV, poxvirus, EBV, vaccinia virus, and hCMV. In some embodiments, the cell is an immune cell, such as a granulocyte, a mast cell, a monocyte, a dendritic cell, a B cell, a T cell, or a natural killer cell. In some embodiments, the cell is a fibroblast. In some embodiments, the cell is a muscle cell. In some embodiments, the cell is a cardiac cell. In some embodiments, the cell is a hepatocyte. In some embodiments, the cell is a human lung progenitor cell (LPC). In some embodiments, the cell is a neuronal cell. In some embodiments, the individual has, or is at risk of developing, a disease, and the virus is useful for the treatment of the disease. In some embodiments, the composition is a pharmaceutical composition, and further comprises a pharmaceutically acceptable carrier. In some embodiments, the individual is a mammal. In some embodiments, the individual is human.

In some embodiments, there is provided a method of delivering a transgene into a cell comprising contacting the cell with a virus delivery complex or nanoparticle as described herein, wherein the virus delivery complex or nanoparticle comprises the transgene packaged in a virus and a CPP comprising the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the virus is a recombinant virus, including recombinant AAV, adenovirus, lentivirus, retrovirus, HSV, poxvirus, EBV, vaccinia virus, and hCMV. In some embodiments, the recombinant virus comprises the transgene for insertion into a cell genome. In some embodiments, the transgene is a therapeutic transgene. In some embodiments, the transgene encodes a protein, such as a therapeutic protein. In some embodiments, the transgene encodes an inhibitory RNA (RNAi), such as an RNAi targeting an endogenous gene, e.g., a disease-associated endogenous gene. In some embodiments, the transgene encodes a CAR. In some embodiments, the complex or nanoparticle comprises one or more viruses comprising a first transgene encoding an RNAi and a second transgene encoding a protein. In some embodiments, the RNAi is a therapeutic RNAi targeting an endogenous gene involved in a disease or condition, and the protein is a therapeutic protein useful for treating the disease or condition. In some embodiments, the therapeutic RNAi targets a disease-associated form of the endogenous gene (e.g., a gene encoding a mutant protein, or a gene resulting in abnormal expression of a protein), and the second transgene is a therapeutic form of the endogenous gene (e.g., the second transgene encodes a wild-type or functional form of the mutant protein, or the second transgene results in normal expression of the protein). In some embodiments, the complex or nanoparticle comprises a first virus comprising the first transgene and a second virus comprising the second transgene. In some embodiments, the complex or nanoparticle comprises a single virus comprising the first transgene and the second transgene. In some embodiments, the contacting of the cell with the complex or nanoparticle is carried out in vivo. In some embodiments, the contacting of the cell with the complex or nanoparticle is carried out ex vivo. In some embodiments, the contacting of the cell with the complex or nanoparticle is carried out in vitro. In some embodiments, the cell is an immortalized cell, such as a cell from a cell line. In some embodiments, the cell is a primary cell, such as a cell from an individual. In some embodiments, the cell is an immune cell, such as a granulocyte, a mast cell, a monocyte, a dendritic cell, a B cell, a T cell, or a natural killer cell. In some embodiments, the T cell is an immortalized T cell, such as a T cell from a T cell line. In some embodiments, the T cell is a primary T cell, such as a T cell of an individual. In some embodiments, the cell is a fibroblast. In some embodiments, the fibroblast is a primary fibroblast, such as a fibroblast of an individual. In some embodiments, the cell is a muscle cell. In some embodiments, the cell is a cardiac cell. In some embodiments, the cell is a hepatocyte. In some embodiments, the hepatocyte is a primary hepatocyte, such as a hepatocyte of an individual. In some embodiments, the cell is a human lung progenitor cell (LPC). In some embodiments, the cell is a neuronal cell. In some embodiments, the virus is useful for the treatment of a disease, such as any of the diseases to be treated described herein (e.g., cancer, diabetes, autoimmune diseases, inflammatory diseases, fibrotic diseases, viral infectious diseases, hereditary diseases, ocular diseases, and aging and degenerative diseases). In some embodiments, the virus is useful for modulating a protein involved in a disease, such as any of the diseases to be treated described herein (e.g., cancer, diabetes, autoimmune diseases, inflammatory diseases, fibrotic diseases, viral infectious diseases, hereditary diseases, ocular diseases, and aging and degenerative diseases). In some embodiments, the cell-penetrating peptide is an ADGN-100 peptide or a VEPEP-3 peptide.

### Methods of Cell Engineering

In some embodiments, there is provided a method of producing an engineered cell, such as an engineered T cell, comprising a method described herein for delivering one or more viruses into a cell. In some embodiments, the method is an improvement over previous methods of producing an engineered cell, such as methods involving the use of electroporation or non-CPP-mediated viral transfection. In some embodiments, the improvement includes, without limitation, increasing the efficiency of the method, reducing costs associated with the method, reducing cellular toxicity of the method, and/or reducing the complexity of the method.

### Methods of Virus Masking

In another aspect of the present application, there is provided a method of masking one or more viruses (such as AAV), comprising combining the one or more viruses with a CPP as described herein, thereby masking the one or more viruses. In some embodiments, the one or more viruses and the CPP form a virus delivery complex or nanoparticle as described herein. In some embodiments, the one or more viruses are immunogenic, and the CPP masks the one or more viruses from being recognized by the immune system of an individual in which the complex or nanoparticle is administered. In some embodiments, the one or more viruses in the complex or nanoparticle are no more than about 99% (such as no more than about any of 95, 90, 85, 80, 75, 70, 65, 60, 55, 50, 45, 40, 35, 30, 25, 20, 15, 10, 5, 4, 3, 2, 10/or less, including any ranges between these values) as immunogenic as the one or more viruses contained in the virus delivery complex or nanoparticle alone.

Because AAV vectors and other gene delivery vectors are often administered directly to a patient, the likelihood of a host immune response is high, as shown by human studies. Preexisting and/or recall responses to the wild-type virus from which the vector is engineered, or to the transgene product itself, can interfere with therapeutic efficacy. Circumventing the immune response to the vector is a major challenge with all vector types. Viral vectors are the most likely to induce an immune response, especially those like adenovirus and AAV, which express immunogenic epitopes within the organism. The first immune response occurring after vector transfer emerges from the innate immune system, mainly consisting of a rapid (few hours) secretion of inflammatory cytokines and chemokines around the administration site. This reaction is high with adenoviral vectors and almost null with AAV. It is noteworthy that plasmid DNA vectors, because of CpG stimulatory islets, also stimulate innate immunity via the stimulation of TLR receptors on leukocytes. Specific immune responses leading to antibody production and T lymphocyte activation also occurs within a few days after vector introduction. Capsid antigens are mostly responsible for specific immunity toward adenoviruses, and are also involved in the response against AAV. Only in the former case, however, can viral gene-encoded proteins also be immunogenic. The pre-existing humoral immunity coming from early infections with wild-type AAV or adenovirus can prevent efficient gene transfer with the corresponding vectors. In all cases, some parameters like route of administration, dose, or promoter type have been extensively described as critical factors influencing vector immunity. Alterations to vector structure have also been extensively performed to circumvent the immune system and thus enhance gene transfer efficiency and safety.

The host immune system represents an important obstacle to be overcome in terms of both safety and efficacy of gene transfer in vivo with AAV vectors. Results in humans undergoing gene transfer indicate that capsid-specific T cell responses directed against transduced cells may limit the duration of transgene expression following AAV gene transfer, and similarly anti-AAV neutralizing antibodies can completely prevent transduction of a target tissue, resulting in lack of efficacy. Anti-AAV neutralizing antibodies are highly prevalent in humans, and the frequency of subjects with detectable titers can reach up to two thirds of the population. The approach to the problem of preexisting humoral immunity to AAV so far has been the exclusion of seropositive subjects, but this solution is far from being optimal. The masking of antigenic sites on AAV vectors, as well as increases in efficiency and reduction in dose, can help to overcome these problems.

It is to be understood that any of the methods described herein can be combined. Thus, for example, a first set of one or more viruses (such as AAV) and a second set of one or more viruses can be delivered into a cell by combining any of the methods described herein for delivering a plurality of virus molecules into a cell. Possible combinations contemplated include combinations of two or more of any of the methods described herein.

### Kits

Also provided herein are kits, reagents, and articles of manufacture useful for the methods described herein. Such kits may contain vials containing the CPPs, assembly molecules and/or other cell-penetrating peptides, separately from vials containing the one or more viruses (such as AAV). At the time of patient treatment, it is first determined what particular pathology is to be treated based on for example, gene expression analysis or proteomic or histological analysis of patient samples. Having obtained those results, the CPPs and any optional assembly molecules and/or cell-penetrating peptides are combined accordingly with the appropriate one or more viruses to result in complexes or nanoparticles that can be administered to the patient for an effective treatment. Thus, in some embodiments, there is provided a kit comprising: 1) a CPP, and optionally 2) one or more viruses. In some embodiments, the kit further comprises assembly molecules and/or other cell-penetrating peptides. In some embodiments, the kit further comprises agents for determining gene expression profiles. In some embodiment, the kit further comprises a pharmaceutically acceptable carrier.

The kits described herein may further comprise instructions for using the components of the kit to practice the subject methods (for example instructions for making the pharmaceutical compositions described herein and/or for use of the pharmaceutical compositions). The instructions for practicing the subject methods are generally recorded on a suitable recording medium. For example, the instructions may be printed on a substrate, such as paper or plastic, etc. As such, the instructions may be present in the kits as a package insert, in the labeling of the container of the kits or components thereof (i.e., associated with the packaging or sub packaging) etc. In some embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet are provided. An example of this embodiment is a kit that includes a web address where the instructions can be viewed and/or from which the instructions can be downloaded. As with the instructions, this means for obtaining the instructions is recorded on a suitable substrate

The various components of the kit may be in separate containers, where the containers may be contained within a single housing, e.g., a box.

### EXEMPLARY EMBODIMENTS

### Embodiment 1

A virus delivery complex for intracellular delivery of a virus comprising a cell-penetrating peptide and the virus, wherein the cell-penetrating peptide is selected from the group consisting of PEP-1 peptides, PEP-2 peptides, PEP-3 peptides, VEPEP-3 peptides, VEPEP-6 peptides, VEPEP-9 peptides, and ADGN-100 peptides.

### Embodiment 2

The virus delivery complex of embodiment 1, wherein the cell-penetrating peptide is a VEPEP-3 peptide.

### Embodiment 3

The virus delivery complex of embodiment 2, wherein the cell-penetrating peptide comprises an amino acid sequence selected from the group consisting of SEQ ID NOs: 1-14.

### Embodiment 4

The virus delivery complex of embodiment 2, wherein the cell-penetrating peptide comprises the amino acid sequence of SEQ ID NO: 75 or 76.

### Embodiment 5

The virus delivery complex of embodiment 1, wherein the cell-penetrating peptide is a VEPEP-6 peptide.

### Embodiment 6

The virus delivery complex of embodiment 5, wherein the cell-penetrating peptide comprises an amino acid sequence selected from the group consisting of SEQ ID NOs: 15-40.

### Embodiment 7

The virus delivery complex of embodiment 5, wherein the cell-penetrating peptide comprises the amino acid sequence of SEQ ID NO: 77.

### Embodiment 8

The virus delivery complex of embodiment 1, wherein the cell-penetrating peptide is a VEPEP-9 peptide.

### Embodiment 9

The virus delivery complex of embodiment 8, wherein the cell-penetrating peptide comprises an amino acid sequence selected from the group consisting of SEQ ID NOs: 41-52.

### Embodiment 10

The virus delivery complex of embodiment 8, wherein the cell-penetrating peptide comprises the amino acid sequence of SEQ ID NO: 78.

### Embodiment 11

The virus delivery complex of embodiment 1, wherein the cell-penetrating peptide is an ADGN-100 peptide.

### Embodiment 12

The virus delivery complex of embodiment 1, wherein the cell-penetrating peptide comprises an amino acid sequence selected from the group consisting of SEQ ID NOs: 53-70.

### Embodiment 13

The virus delivery complex of embodiment 11, wherein the cell-penetrating peptide comprises the amino acid sequence of SEQ ID NO: 79 or 80.

### Embodiment 14

The virus delivery complex of embodiment 1, wherein the cell-penetrating peptide is a PEP-1, PEP-2, or PEP-3 peptide.

### Embodiment 15

The virus delivery complex of embodiment 14, wherein the cell-penetrating peptide comprises the amino acid sequence of any one of SEQ ID NOs: 71-73.

### Embodiment 16

The virus delivery complex of embodiment 1, wherein the cell-penetrating peptide is covalently linked to the virus.

### Embodiment 17

The virus delivery complex of any one of embodiments 1-16, wherein the cell-penetrating peptide further comprises one or more moieties covalently linked to the N-terminus of the cell-penetrating peptide, and wherein the one or more moieties are selected from the group consisting of an acetyl, a fatty acid, a cholesterol, a poly-ethylene glycol, a nuclear localization signal, nuclear export signal, an antibody, a polysaccharide and a targeting molecule.

### Embodiment 18

The virus delivery complex of embodiment 17, wherein the cell-penetrating peptide comprises an acetyl group covalently linked to its N-terminus.

### Embodiment 19

The virus delivery complex of any one of embodiments 1-18, wherein the cell-penetrating peptide further comprises one or more moieties covalently linked to the C-terminus of the cell-penetrating peptide, and wherein the one or more moieties are selected from the group consisting of a cysteamide, a cysteine, a thiol, an amide, a nitrilotriacetic acid optionally substituted, a carboxyl, a linear or ramified C1-C6 alkyl optionally substituted, a primary or secondary amine, an osidic derivative, a lipid, a phospholipid, a fatty acid, a cholesterol, a poly-ethylene glycol, a nuclear localization signal, nuclear export signal, an antibody, a polysaccharide and a targeting molecule.

### Embodiment 20

The virus delivery complex of embodiment 19, wherein the cell-penetrating peptide comprises a cysteamide group covalently linked to its C-terminus.

### Embodiment 21

The virus delivery complex of any one of embodiments 1-20, wherein at least some of the cell-penetrating peptides in the virus delivery complex are linked to a targeting moiety by a linkage.

### Embodiment 22

The virus delivery complex of embodiment 21, wherein the linkage is covalent.

### Embodiment 23

The virus delivery complex of any one of embodiments 1-22, wherein the virus is a recombinant virus.

### Embodiment 24

The virus delivery complex of embodiment 23, wherein the recombinant virus comprises a transgene for insertion into a cell genome.

### Embodiment 25

The virus delivery complex of embodiment 23 or 24, wherein the recombinant virus is recombinant adeno-associated virus (AAV), adenovirus, lentivirus, retrovirus, herpes simplex virus (HSV), poxvirus, Epstein-Barr virus (EBV), vaccinia virus, or human cytomegalovirus (hCMV).

### Embodiment 26

The virus delivery complex of embodiment 25, wherein the virus is recombinant AAV.

### Embodiment 27

The virus delivery complex of embodiment 26, wherein the AAV is AAV1, AAV2, AAV3, AAV4, AAV5, AAV6, AAV7, AAV8, AAV9, AAV 10, AAV11, or AAV12.

### Embodiment 28

The virus delivery complex of embodiment 26, wherein the AAV is pseudotyped, comprising a capsid and genome derived from different viral serotypes.

### Embodiment 29

The virus delivery complex of any one of embodiments 1-28, wherein the molar ratio of the cell-penetrating peptide to the virus (in Vg, pfu, or MOI) is between about 1:1 and about 1×108:1.

### Embodiment 30

The virus delivery complex of any one of embodiments 1-29, wherein the average diameter of the virus delivery complex is between about 20 nm and about 1000 nm.

31. A nanoparticle comprising a core comprising the virus delivery complex of any one of embodiments 1-30.

### Embodiment 32

The nanoparticle of embodiment 31, wherein the core further comprises one or more additional virus delivery complexes according to any one of embodiments 1-30.

### Embodiment 33

The nanoparticle of embodiment 31 or 32, wherein at least some of the cell-penetrating peptides in the nanoparticle are linked to a targeting moiety by a linkage.

### Embodiment 34

The nanoparticle of any one of embodiments 31-33, wherein the core is coated by a shell comprising a peripheral cell-penetrating peptide.

### Embodiment 35

The nanoparticle of embodiment 34, wherein the peripheral cell-penetrating peptide is selected from the group consisting of PEP-1 peptides. PEP-2 peptides. PEP-3 peptides, VEPEP-3 peptides, VEPEP-6 peptides, VEPEP-9 peptides, and ADGN-100 peptides.

### Embodiment 36

The nanoparticle of embodiment 35, wherein the peripheral cell-penetrating peptide comprises an amino acid sequence selected from the group consisting of SEQ ID NOs: 1-80.

### Embodiment 37

The nanoparticle of any one of embodiments 34-36, wherein at least some of the peripheral cell-penetrating peptides in the shell are linked to a targeting moiety by a linkage.

### Embodiment 38

The nanoparticle of embodiment 33 or 37, wherein the linkage is covalent.

### Embodiment 39

The nanoparticle of any one of embodiments 31-38, wherein the average diameter of the nanoparticle is between about 20 nm and about 1000 nm.

### Embodiment 40

A pharmaceutical composition comprising the virus delivery complex of any one of embodiments 1-30 or the nanoparticle of any one of embodiments 31-39, and a pharmaceutically acceptable carrier.

### Embodiment 41

The pharmaceutical composition of embodiment 40, wherein the virus delivery complex or nanoparticle comprises a virus comprising a transgene encoding a therapeutic protein.

### Embodiment 42

The pharmaceutical composition of embodiment 40, wherein the virus delivery complex or nanoparticle comprises a virus comprising a transgene encoding an inhibitory RNA (RNAi).

### Embodiment 43

The pharmaceutical composition of embodiment 40, wherein the virus delivery complex or nanoparticle comprises one or more viruses comprising a first transgene encoding a therapeutic protein and a second transgene encoding an RNAi.

### Embodiment 44

The pharmaceutical composition of embodiment 43, wherein the virus delivery complex or nanoparticle comprises a first virus comprising a first transgene encoding a therapeutic protein and a second virus comprising a second transgene encoding an RNAi.

### Embodiment 45

The pharmaceutical composition of embodiment 40, wherein the virus delivery complex or nanoparticle comprises a virus comprising a transgene encoding a chimeric antigen receptor (CAR).

### Embodiment 46

A method of preparing the virus delivery complex of any one of embodiments 1-30, comprising combining the cell-penetrating peptide with the one or more viruses, thereby forming the virus delivery complex.

### Embodiment 47

The method of embodiment 46, wherein the cell-penetrating peptide and the virus (in Vg, pfu, or MOI) are combined at a ratio from about 1:1 to about 1×108:1, respectively.

### Embodiment 48

A method of delivering one or more viruses into a cell, comprising contacting the cell with the virus delivery complex of any one of embodiments 1-30 or the nanoparticle of any one of embodiments 31-39, wherein the virus delivery complex or the nanoparticle comprises the one or more viruses.

### Embodiment 49

The method of embodiment 48, wherein the contacting of the cell with the virus delivery complex or nanoparticle is carried out in vivo.

### Embodiment 50

The method of embodiment 48, wherein the contacting of the cell with the virus delivery complex or nanoparticle is carried out ex vivo.

### Embodiment 51

The method of embodiment 48, wherein the contacting of the cell with the virus delivery complex or nanoparticle is carried out in vitro.

### Embodiment 52

The method of any one of embodiments 48-51, wherein the cell is a granulocyte, a mast cell, a monocyte, a dendritic cell, a B cell, a T cell, a natural killer cell, a fibroblast, a muscle cell, a cardiac cell, a hepatocyte, a lung progenitor cell, or a neuronal cell.

### Embodiment 53

The method of embodiment 52, wherein the cell is a T cell.

### Embodiment 54

The method of embodiment 52 or 53, wherein the virus targets a sequence in a gene selected from the group consisting of PD-1, PD-L1, PD-L2, TIM-3, BTLA, VISTA, LAG-3, CTLA-4, TIGIT, 4-1BB, OX40, CD27, TIM-1, CD28, HVEM, GITR, and ICOS.

### Embodiment 55

The method of any one of embodiments 48-54, wherein the virus delivery complex or nanoparticle comprises a virus comprising a transgene encoding a therapeutic protein.

### Embodiment 56

The method of any one of embodiments 48-54, wherein the virus delivery complex or nanoparticle comprises a virus comprising a transgene encoding an inhibitory RNA (RNAi).

### Embodiment 57

The method of any one of embodiments 48-54, wherein the virus delivery complex or nanoparticle comprises one or more viruses comprising a first transgene encoding a therapeutic protein and a second transgene encoding an RNAi.

### Embodiment 58

The method of embodiments 57, wherein the virus delivery complex or nanoparticle comprises a first virus comprising a first transgene encoding a therapeutic protein and a second virus comprising a second transgene encoding an RNAi.

### Embodiment 59

The method of any one of embodiments 48-54, wherein the virus delivery complex or nanoparticle comprises a virus comprising a transgene encoding a chimeric antigen receptor (CAR).

### Embodiment 60

A method of treating a disease in an individual comprising administering to the individual an effective amount of the pharmaceutical composition of any one of embodiments 40-45.

### Embodiment 61

The method of embodiment 60, wherein the disease is selected from the group consisting of cancer, diabetes, autoimmune diseases, hematological diseases, cardiac diseases, vascular diseases, inflammatory diseases, fibrotic diseases, viral infectious diseases, hereditary diseases, ocular diseases, liver diseases, lung diseases, muscle diseases, enzyme deficiency diseases, lysosomal storage diseases, neurological diseases, kidney diseases, aging and degenerative diseases, and diseases characterized by cholesterol level abnormality.

### Embodiment 62

The method of embodiment 61, wherein the disease is cancer.

### Embodiment 63

The method of embodiment 62, wherein the cancer is a solid tumor, and wherein the pharmaceutical composition comprises a virus delivery complex or nanoparticle comprising one or more viruses that modulates the expression of one or more proteins selected from the group consisting of growth factors and cytokines, cell surface receptors, signaling molecules and kinases, transcription factors and other modulators of transcription, regulators of protein expression and modification, tumor suppressors, and regulators of apoptosis and metastasis.

### Embodiment 64

The method of embodiment 63, wherein the cancer is cancer of the liver, lung, or kidney.

### Embodiment 65

The method of embodiment 62, wherein the cancer is a hematological malignancy, and wherein the pharmaceutical composition comprises a virus delivery complex or nanoparticle comprising one or more viruses that modulates the expression of one or more proteins selected from the group consisting of growth factors and cytokines, cell surface receptors, signaling molecules and kinases, transcription factors and other modulators of transcription, regulators of protein expression and modification, tumor suppressors, and regulators of apoptosis and metastasis.

### Embodiment 66

The method of embodiment 61, wherein the disease is a viral infection disease, and wherein the pharmaceutical composition comprises a virus delivery complex or nanoparticle comprising one or more viruses that modulates the expression of one or more proteins involved in the viral infectious disease development and/or progression.

### Embodiment 67

The method of embodiment 61, wherein the disease is a hereditary disease, and wherein the pharmaceutical composition comprises a virus delivery complex or nanoparticle comprising one or more viruses that modulates the expression of one or more proteins involved in the hereditary disease development and/or progression.

### Embodiment 68

The method of embodiment 61, wherein the disease is an aging or degenerative disease, and wherein the pharmaceutical composition comprises a virus delivery complex or nanoparticle comprising one or more viruses that modulates the expression of one or more proteins involved in the aging or degenerative disease development and/or progression.

### Embodiment 69

The method of embodiment 61, wherein the disease is a fibrotic or inflammatory disease, and wherein the pharmaceutical composition comprises a virus delivery complex or nanoparticle comprising one or more viruses that modulates the expression of two or more proteins involved in the fibrotic or inflammatory disease development and/or progression.

### Embodiment 70

The method of any one of embodiments 60-69, wherein the individual is human.

### Embodiment 71

A kit comprising a composition comprising the virus delivery complex of any one of embodiments 1-30 and/or the nanoparticle of any one of embodiments 31-39.

### EXAMPLES

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### Materials and Methods

### Cell Penetrating Peptides:

The following peptides were used:

PEP-1:
(SEQ ID NO: 71)
KETWWETWWTEWSQPKKKRKV
PEP-2:
(SEQ ID NO: 72)
KETWFETWFTEWSQPKKKRKV
VEPEP-3a:
(SEQ ID NO: 75)
βAKWFERWFREWPRKRR
VEPEP-3b:
(SEQ ID NO: 76)
βAKWWERWWREWPRKRR
VEPEP-6:
(SEQ ID NO: 77)
βALWRALWRLWRSLWRLLWKA
VEPEP-9:
(SEQ ID NO: 78)
βALRWWLRWASRWFSRWAWWR
ADGN-100a:
(SEQ ID NO: 79)
βAKWRSAGWRWRLWRVRSWSR
ADGN-100b:
(SEQ ID NO: 80)
βAKWRSALYRWRLWRVRSWSR
pANT:
(SEQ ID NO: 82)
RQIKIWFQNRRMKWKKC
TAT-HA2:
(SEQ ID NO: 83)
CRRRQRRKKRGGDIMGEWGNEIFGAIAGFLG

Stock solutions of peptides were prepared at 2 mg/mL in distilled water or 5% DMSO and sonicated for 10 min in a water bath sonicator then diluted just before use.

### Cell Lines

Several cell lines were used, including stable EGFP expressing cell lines (GFP-U2OS, EGFP-JURKAT T, EGFP-HEK) as well as U2OS (ATCC® HTB-96™), primary human fibroblasts, Hep G2 (ATCC® HB-8065™), Human Embryonic Kidney (HEK293) (ATCC® CRL-1573™), Human Myelogenous Leukemia K562 cells (ATCC® CCL243 ™), Jurkat T cells (ATCC® TIB-152™), human ESCs (H9), and mouse ESCs (ESF 158). Cells were obtained from the American Type Culture Collection [ATCC].

### Example 1: Enhancing Gene Delivery in Cultured Cells of Adeno-Associated Viruses by Cell Penetrating Peptides

Adeno-associated virus (AAV) has been largely evaluated for in vivo gene therapy and clinical trial. AAV leads to the establishment of a long-term gene expression in both dividing and non-dividing cells with limited side effects. However, AAV clinical applications remain limited by the fact that AAV can infect only a small number of permissive cell types and by a single dose administration due to the rapid emergence in vivo of viral antigens. Cell-penetrating peptides (CPPs) provide a safe, efficient, and non-invasive mode of transport for various cargos into cells, they have been developed as vectors for the delivery of genetic and biologic products in recent years Cell-penetrating peptides (CPPs) can cross cellular membranes in a non-toxic fashion, improving the intracellular delivery of various molecular cargos including nanoparticles, small molecules, siRNA, protein and plasmid DNA.

In the present example, we have investigated the impact of Cell Penetrating peptides on AAV-2 and AAV-6 mediated gene delivery in different cell lines. We have compared 2 ADGN-related CPPs (PEP-1 and PEP-2) with well know CPPs such as Antp and TAT-HA2. We demonstrated that both CPPs form stable complexes with AAV and significantly enhanced AAV2 and AAV6-mediated transduction into nonpermissive cells such as human hepatocyte HepG2, human fibroblast (HS68) and HUVEC.

Cell Penetrating Peptides (CPP's) Significantly Increase Virus Mediated Gene Delivery in Cultured Cells

Viruses:

Adeno-associated viruses (AAV-2 and AAV-6) encoding for Green Fluorescent Protein (AAV-GFP) or for betagalactosidase (AAV-βGA1) were produced in permissive cell type HEK 293 cells.

Peptides:

Peptides were obtained by solid phase synthesis

Cell Cultured:

Cells (HS68, HepG2 and HUVEC) were cultured in Dulbecco's Modified Eagle's Medium (DMEM), supplemented with 2 mM glutamine, 1% antibiotics (streptomycin 10,000 μg/mL, penicillin, 10,000 IU/mL) and 10% (w/v) foetal calf serum (FCS), at 37° C. in a humidified atmosphere containing 5% CO₂.

### Results

### Evaluation of the Impact of CPPs on the Infectivity of AAVs at Reduced Titers

AAV encoding green fluorescent protein (AAV2-GFP) or AAV encoding beta galactosidase (AAV-6-βGal) at MOI of 300 were preincubated in PBS with increasing concentration of CPPs ranging from 0.1 to 500 μM for 30 min. AAV alone or CPP/AAV complexes were added to cultured cells in a 24 well plate format. Cells were treated with either free AAV or CPP/AAV complexes for 4 h, and then the medium was replaced with fresh medium. Viral mediated transgene expression was monitored 2 days post-infection in cell lines HS-68 and HepG2 for AAV-GFP and HUVEC for AAV-βGal. GFP expressing cells were detected by flow cytometry and β-Galactosidase activity was determined in cell lysates.

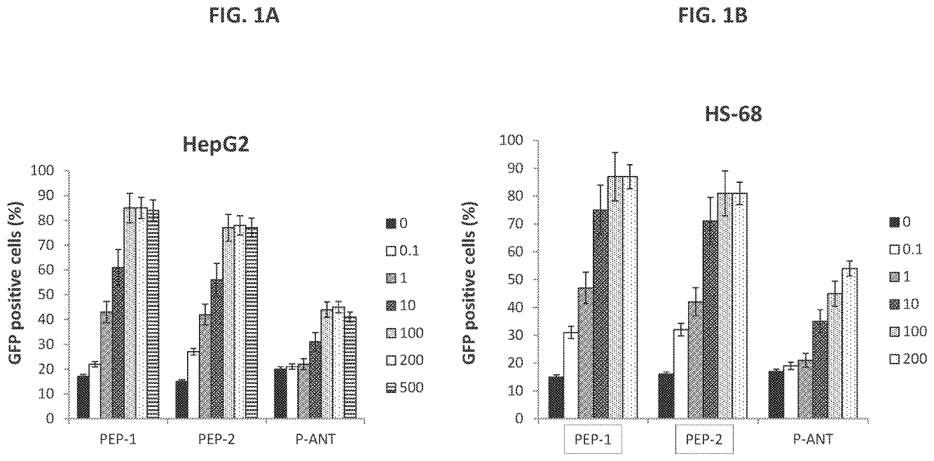

As reported in FIGS. 1A and 1B, CPPs significantly increase AAV entry and gene expression in HepG2 and HS68 cell lines in a dose dependent manner and at reduced titer of virus. Free AAV leads to 12-15% GFP-positive cells and the used of 100 μM concentration of PEP-1 or PEP-2 increases by about 8-fold the level of GFP-positive cells for both cell lines. PEP-1 and PEP-2 are 2-fold more potent than pANT (penetratin).

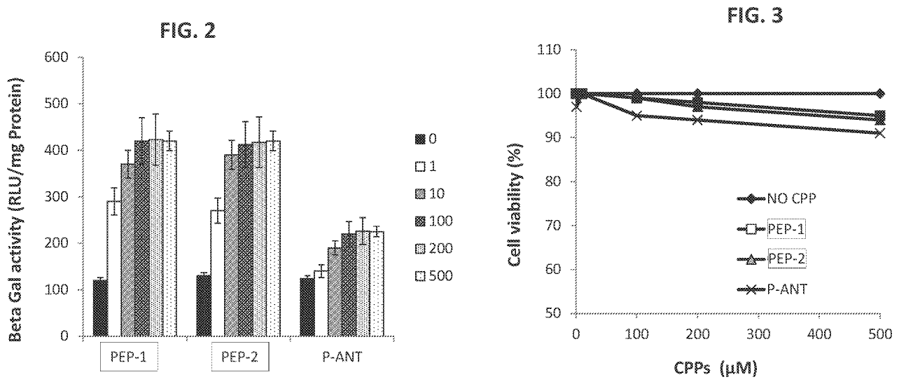

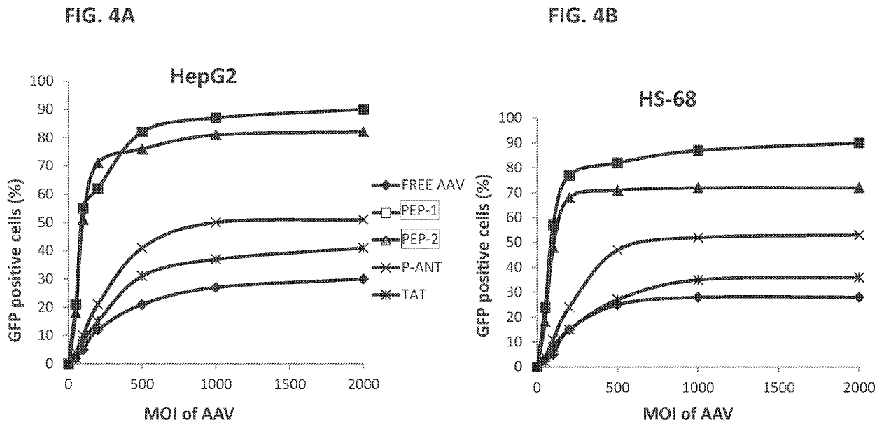

As reported in FIG. 2 CPPs significantly increase AAV entry and gene expression in HUVEC in a dose dependent manner and at reduced titers of virus. Optimal responses are obtained for PEP-1 and PEP-2 concentration of 100 μM. PEP-1 and PEP-2 increase by about 4-fold the level of beta Galactosidase expression in comparison to free AAV. PEP-1 and PEP-2 are 2-fold more potent than pANT (penetratin).

The cytoxicity of the AAV-CPP complexes was evaluated on HUVEC cell lines. AAV encoding beta galactosidase (AAV-6-βGal) at MOI of 300 were preincubated in PBS with increasing concentration of CPPs ranging from 1 to 500 μM for 30 min. Cells were treated with either free AAV (No CPP) or CPP/AAV complexes for 4 h, then the medium was replaced with fresh medium. Cytotoxicity of AAV-CPP complexes was determined using the XTT assay after 2 days. As reported in FIG. 3, no toxicity of the CPP/AAV complexes was observed at concentration of 100 μM. A low cytotoxicity of 5% for PEP-1 and PEP-2 and 8% for Pant were obtained for a CPP concentration of 500 μM.

### Evaluation of the Impact of CPPs on the Titer Necessary for Virus Mediated Gene Expression

A fixed 200 μM concentration of peptides (PEP-1, PEP-2, Penetratin (P-ANT) and TAT) were preincubated with increasing MOI (up to 2000) of AAV-2 encoding green fluorescent protein (AAV-GFP). HS 68 and HepG2 cells were treated with either free AAV or AAV:CPP complexes for 4 h, then the medium was replaced with fresh medium. GFP expression was analyzed 2 days after infection and GFP expressing cells were quantified by flow cytometry.

As reported in FIGS. 3A and 3B, the preincubation of CPPs significantly increase AAV infectivity aby 20-fold at low titers titer of AAV. The level of GFP-positive cells is increased by 5-fold in the presence of PEP-1 and PEP-2. In contrast, no chance in infectivity is observed with TAT peptide and an increase of 5-fold is observed with pANT peptide.

### Example 2A: Evaluation of the Impact of CPPs on the Infectivity of Different AAV Serotypes at Reduced Titers

11 AAV serotypes have been identified so far and all serotypes are able to infect cells from multiple diverse tissue types. However, infectivity varied from one serotype to another one and tissue specificity is determined by the capsid serotype (as reported in FIG. 3 from Vance et al, 2016). Therefore pseudotyping of AAV vectors or using CPPs to alter their tropism range will likely be important to their use in therapy. In this example different AAV serotypes including AAV1, AAV2, AAV5, AAV6, AAV8, and AAV9 are challenged against various cell types (e.g., human hepatocytes (HepG2) and human neuronal cells (HCN2)) in the presence or in the absence of CPPs, including, e.g., ADGN-103a (SEQ ID NO: 75), ADGN-103b (SEQ ID NO: 76), ADGN-100 (SEQ ID NO: 79), ADGN-109 (SEQ ID NO: 78), ADGN-106 (SEQ ID NO: 77), PEP-1 (SEQ ID NO: 71), PEP-2 (SEQ ID NO: 72), CADY (SEQ ID NO: 81), and TAT-HA2 (SEQ ID NO: 83) (ADGN-103 is used herein interchangeably with VEPEP-3; ADGN-106 is used herein interchangeably with VEPEP-6; and ADGN-109 is used herein interchangeably with VEPEP-9).

AAV encoding green fluorescent protein, e.g., at low MOI of 300 (1×10⁷ PFU), are preincubated in PBS with increasing concentration of CPPs ranging from, e.g., 0.1 μM to 500 μM for 30 min. AAV alone or CPP/AAV complexes are added to cultured cells, e.g., in a 24 well plate format. Cells are treated with either free AAV or CPP/AAV complexes (e.g., for 4 hours), and then the medium is replaced with fresh medium. Viral-mediated transgene expression is monitored (e.g., for 2 days post-infection in HepG2 for AAV-GFP). GFP expressing cells are detected, e.g., by flow cytometry.

### Example 2B: Evaluation of the Impact of CPPs on the Infectivity of Different AAV Serotypes at Reduced Titers in HepG2 and HCN2 Cells

In this example different AAV serotypes including AAV 1, AAV2, AAV5, AAV6, AAV8 and AAV9 were tested against human hepatocytes (HepG2) and human neuronal cells (HCN2) in the presence or in the absence of CPPs including ADGN-103a, ADGN-103b, ADGN-100, ADGN-109, ADGN-106, PEP-1, PEP-2, CADY, and TAT-HA2.

AAV-1, AAV-2, AAV-5, and AAV-6 viruses encoding green fluorescent protein were obtained from Cell Biolabs Inc and AAV-8 from Vector Biolabs. Stock solutions of virus were obtained at 1χ10¹³ GC/ml in PBS/0.01% and diluted 50-fold to 2×10¹¹ GC/ml before use.

AAV-1, AAV-2, AAV-5, and AAV6 encoding green fluorescent protein were used at low MOI of 500 (1-2×10⁷ PFU) and AAV-8 at MOI of 1000 (2-4×10⁷ PFU). HepG2 and HCN2 cells (1 10⁶ cells per well) were cultured in a 24 well plate culture dish format. For MOI of 1000, 5 μl of AAV virus diluted solutions were preincubated in PBS with increasing concentration of CPPs ranging from 0.1 to 200 μM for 30 min. For MOI of 500, 2.5 μl of AAV virus diluted solutions were preincubated in PBS with increasing concentration of CPPs ranging from 0.1 to 200 μM for 30 min. 200 μl of AAV or CPP/AAV complex solutions were added to cultured cells (1×10⁶ cells per well). Cells are treated with either free AAV or CPP/AAV complexes for 4 h. and then the medium is replaced with fresh medium.

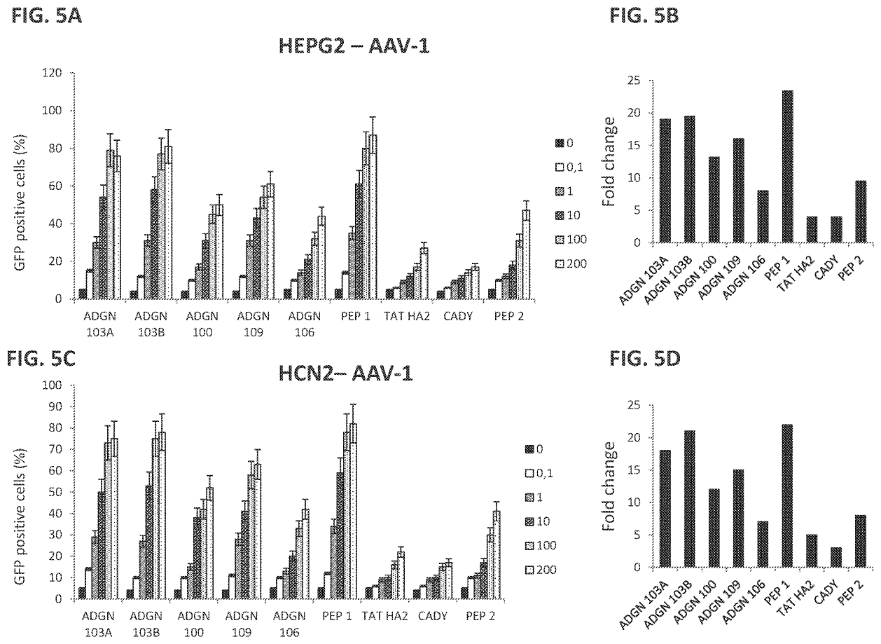

Viral mediated transgene expression was monitored by flow cytomety 2 days post-infection in HepG2 and Human neuronal HCN2. The percentage of GFP expressing cells were detected by flow cytometry and the increase of AAV infectivity was calculated based on the number of GFP-positive cells. Results are reported in FIGS. 5A-5D, 6A-6D, 7A-7D, 8A-8D, and 9A-9D.

As reported in FIGS. 5A-5D, all tested CPPs enhanced AAV 1-mediated gene expression in non-permissive HepG2 and HCN2 cells. In HepG2, at the highest concentration of 200 μM CPPs, ADGN-103a, ADGN-103b, and PEP-1 increased AAV-1 efficiency by 20-fold up to 23-fold for PEP-1. Increases by 16-fold and 13-fold were obtained for ADGN-109 and ADGN-100, respectively. In comparison, ADGN-106 and PEP-2 increased AAV-1 efficiency by 7- to 8-fold, and TAT-HA2 and CADY by only 3- to 4-fold (FIG. 5B). These results suggest that ADGN-103a and ADGN-103b are about 5- to 6-fold more potent than the CPP TAT-HA2. The number of cells expressing GFP increased from 5% in the absence of CPP to 87%, 81%, 76%, 61%, and 50% using 200 μM of PEP-1, ADGN-103b, ADGN-103a, ADGN-109, and ADGN-100, respectively. In contrast, only 27% GFP-positive cells were obtained using TAT-HA2 or CADY peptide (FIG. 5A).

In human neuronal cells HCN2, at the highest concentration of 200 μM CPPs, ADGN-103a, ADGN-103b, and PEP-1 increased AAV-1 efficiency by 18-fold up to 22-fold for PEP-1. Increases by 15-fold and 12-fold were obtained for ADGN-109 and ADGN-100, respectively. In comparison, ADGN-106 and PEP-2 increased AAV-1 efficiency by 7-fold, and TAT-HA2 and CADY by only 3-fold (FIG. 5D). The number of cells expressing GFP increased from 5% in the absence of CPP to 82%, 78%, 75%, 62%, and 52% using 200 μM of PEP-1, ADGN-103b, ADGN-103a, ADGN-109, and ADGN-100, respectively. In contrast, only 22% GFP-positive cells were obtained using TAT-HA2 or CADY peptide (FIG. 5C).

For both cell lines, a significant impact of ADGN-103b, ADGN-103a, ADGN-109, and ADGN-100 on AAV-1 infectivity was clear even at 1 μM concentration, and about 60% of the final improvement obtained with 200 μM was already observed at a 10 μM concentration of CPP (FIGS. 5A and 5C).

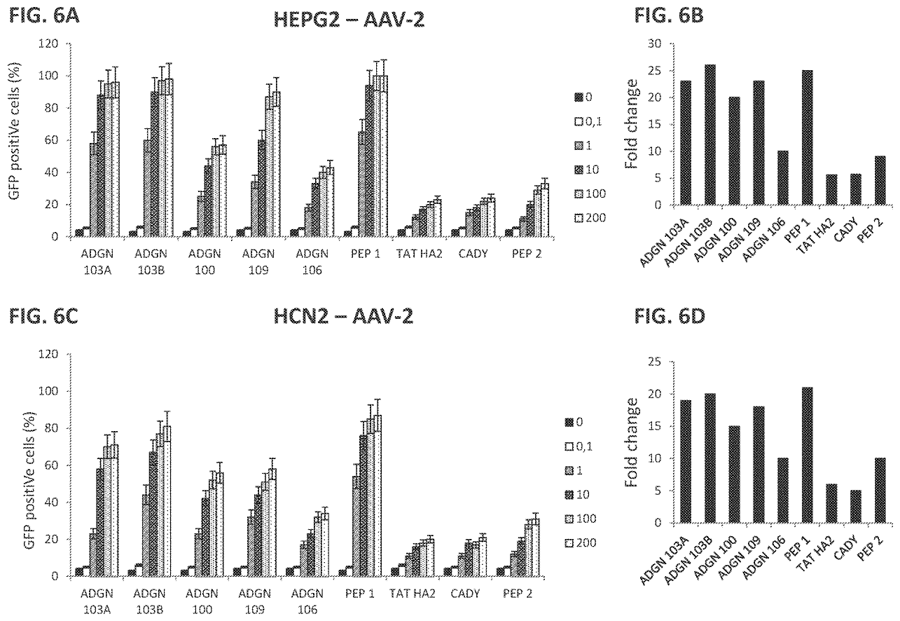

As reported in FIGS. 6A-6D, all tested CPPs enhance AAV-2 mediated gene expression in non-permissive HepG2 and HCN2 cells. In HepG2, at the highest concentration of 200 μM CPPs. ADGN-103a, ADGN-103b, ADGN-109, and PEP-1 increased AAV-2 efficiency by 23-fold up to 25-fold in the following order PEP1>ADGN-103b>ADGN-103a>ADGN-109. An increase by 20-fold was obtained for ADGN-100. In comparison, ADGN-106 and PEP-2 increased AAV-2 efficiency by 9- to 10-fold, and TAT-HA2 and CADY by only 5-fold (FIG. 6B). These results suggest that ADGN-103a and ADGN-103b are about 5- to 6-fold more potent than the CPP TAT-HA2. The number of cells expressing GFP increased from 4% in the absence of CPP to 98%, 96%, 98%, 90%, and 57% using 200 μM of PEP-1, ADGN-103b, ADGN-103a, ADGN-109, and ADGN-100, respectively. In contrast, only 23% GFP-positive cells were obtained using TAT-HA2 or CADY peptide (FIG. 6A).

In human neuronal cells HCN2, at the highest concentration of 200 μM CPPs, ADGN-103a, ADGN-103b, PEP-1, and ADGN-109 increased AAV-2 efficiency by 20-fold up to 21-fold for PEP-1 in the following order PEP1>ADGN-103b>ADGN-103a>ADGN-109. An increase by 15-fold was obtained for ADGN-100. In comparison, ADGN-106 and PEP-2 increased AAV-2 efficiency by 7-fold, and TAT-HA2 and CADY by only 3-fold (FIG. 6D). The number of cells expressing GFP increased from 4% in the absence of CPP to 87%, 81%, 71%, 58%, and 59% using 200 μM of PEP-1, ADGN-103b, ADGN-103a, ADGN-109, and ADGN-100, respectively. In contrast, only 20% GFP-positive cells were obtained using TAT-HA2 or CADY peptide (FIG. 6C).

For both cell lines, a significant impact of ADGN-103b, ADGN-103a, ADGN-109, and ADGN-100 on AAV-2 infectivity was clear even at 1 μM concentration, and about 70% of the final improvement obtained with 200 μM was observed at a 10 μM concentration of CPP (FIGS. 6A and 6C).

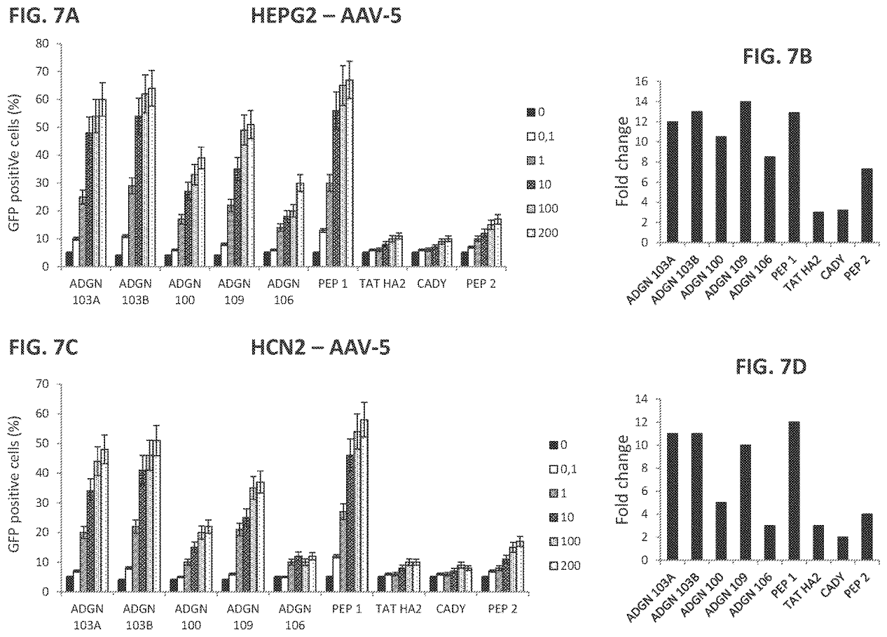

As reported in FIGS. 7A-7D, all tested CPPs enhanced AAV5-mediated gene expression in non-permissive HepG2 and HCN2 cells. In HepG2, at the highest concentration of 200 μM CPPs, ADGN-103a, ADGN-103, PEP-1, and ADGN-109 increased AAV-5 efficiency by 12- to 14-fold. An increase by 10-fold was obtained for ADGN-100. In comparison, ADGN-106 and PEP-2 increased AAV-5 efficiency by 7- to 8-fold, and TAT-HA2 and CADY by only 3-fold (FIG. 7B). These results suggest that ADGN-103a and ADGN-103b are about 4- to 5-fold more potent than the CPP TAT-HA2. The number of cells expressing GFP increased from 5% in the absence of CPP to 67%, 64%, 60%, 51%, and 39% using 200 μM of PEP-1, ADGN-103b, ADGN-103a, ADGN-109, and ADGN-100, respectively. In contrast, only 11% GFP-positive cells were obtained using TAT-HA2 or CADY peptide (FIG. 7A).

In human neuronal cells HCN2, at the highest concentration of 200 μM CPPs, ADGN-103a, ADGN-103b, ADGN-109, and PEP-1 increased AAV-5 efficiency by 11-fold up to 12-fold for PEP-1. An increase by 5-fold was obtained for ADGN-100. In comparison, ADGN-106 and PEP-2 increased AAV-5 efficiency by 4-fold, and TAT-HA2 and CADY by only 2-fold (FIG. 7D). The number of cells expressing GFP increased from 5% in the absence of CPP to 52%, 48%, 45%, 37%, and 22% using 200 μM of PEP-1i ADGN-103b, ADGN-103a, ADGN-109, and ADGN-100, respectively. In contrast, only 10% GFP-positive cells were obtained using TAT-HA2 or CADY peptide (FIG. 7C).

For both cell lines, a significant impact of ADGN-103b, ADGN-103a, ADGN-109, and ADGN-100 on AAV-5 infectivity was clear even at 1 μM concentration, and about 50% of the final improvement obtained at 200 μM was already observed at a 10 μM concentration.

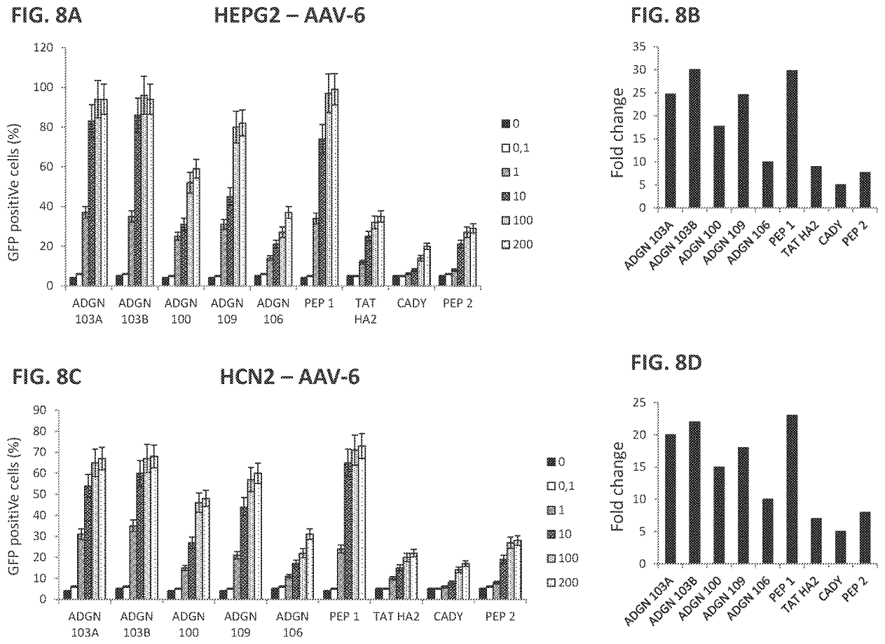

As reported in FIGS. 8A-8D, all tested CPPs enhanced AAV6-mediated gene expression in non-permissive HepG2 and HCN2 cells. In HepG2, at the highest concentration of 200 μM CPPs, ADGN-103a, ADGN-103b, ADGN-109, and PEP-1 increased AAV-6 efficiency by 25-fold up to 30-fold for ADGN-103b in the following order ADGN-103b>PEP1>ADGN-103a>ADGN-109. An increase by 17-fold was obtained for ADGN-100. In comparison, ADGN-106 and TAT-HA2 increased AAV-6 efficiency by about 10-fold, and PEP-2 and CADY by only 7- to 5-fold (FIG. 8B). The number of cells expressing GFP increased from 5% in the absence of CPP to 99%, 98%, 94%, 82%, and 59% using 200 μM of PEP-1, ADGN-103b, ADGN-103a. ADGN-109, and ADGN-100, respectively. In contrast, only 35% and 29% GFP-positive cells were obtained using TAT-HA2 and CADY peptides, respectively (FIG. 8A).

In human neuronal cells HCN2, at the highest concentration of 200 μM CPPs, ADGN-103a, ADGN-103b, and PEP-1 increased AAV-6 efficiency by 18-fold up to 22-fold for ADGN-103b in the following order ADGN-103b>PEP1>ADGN-103a>ADGN-109. An increase by 15-fold was obtained for ADGN-100. In comparison, ADGN-106 and PEP-2 increased AAV-6 efficiency by 7-fold, and TAT-HA2 and CADY by only 3-fold (FIG. 8D). The number of cells expressing GFP increased from 4% in the absence of CPP to 72%, 67%, 65%, 62%, and 48% using 2001M of PEP-1, ADGN-103b, ADGN-103a, ADGN-109, and ADGN-100, respectively. In contrast, only 22% and 18% GFP-positive cells were obtained using TAT-HA2 and CADY peptide, respectively (FIG. 8C).

For both cell lines, a significant impact of ADGN-103b, ADGN-103a, ADGN-109, and ADGN-100 on AAV-6 infectivity was clear even at 1 μM concentration, and about 70% of the final improvement obtained at 200 μM was already observed at a 10 μM concentration.

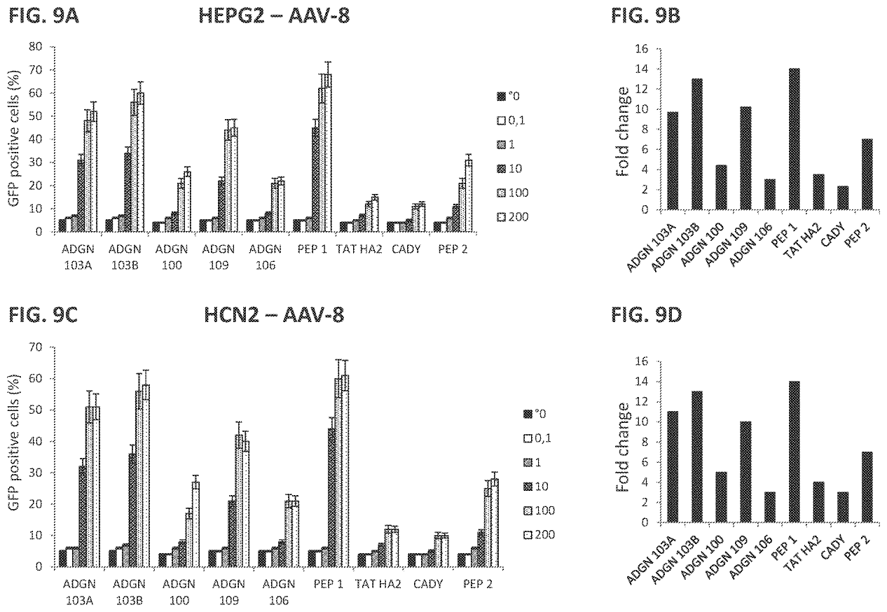

As reported in FIGS. 9A-9D, all tested CPPs enhanced AAV-8 mediated gene expression in non-permissive HepG2 and HCN2 cells. In HepG2, at the highest concentration of 200 μM CPPs, ADGN-103a, ADGN-103b, ADGN-109, and PEP-1 increased AAV-8 efficiency by 10-fold up to 14-fold for PEP-1, in the following order PEP1>ADGN-103b>ADGN-103a>ADGN-109. In comparison, ADGN-106, ADGN-100, TAT-HA2, and CADY increased AAV-8 efficiency by only 3- to 5-fold (FIG. 9B). The number of cells expressing GFP increased from 5% in the absence of CPP to 68%, 60%, 52%, 45%, and 26% using 200 μM of PEP-1, ADGN-103b, ADGN-103a, ADGN-109, and ADGN-100, respectively. In contrast, only 15% GFP-positive cells were obtained using TAT-HA2 or CADY peptide (FIG. 9A).

In human neuronal cells HCN2, at the highest concentration of 200 μM CPPs, ADGN-103a, ADGN-103b, ADGN-109, and PEP-1 increased AAV-8 efficiency by 12-fold up to 14-fold for PEP-1, in the following order PEP1>ADGN-103b>ADGN-103a>ADGN-109. In comparison, ADGN-106, ADGN-100, TAT-HA2, and CADY increased AAV-8 efficiency by only 4- to 5-fold (FIG. 9D). The number of cells expressing GFP increased from 5% in the absence of CPP to 61%, 58%, 49%, 44%, and 23% using 200 μM of PEP-1, ADGN-103b, ADGN-103a, ADGN-109, and ADGN-100, respectively. In contrast, only 12% GFP-positive cells were obtained using TAT-HA2 or CADY peptide (FIG. 9C).

For both cell lines, a significant impact of ADGN-103b, ADGN-103a, ADGN-109, and ADGN-100 on AAV-8 infectivity was clear even at 1 μM concentration, and about 60% of the final improvement obtained at 200 μM was already observed at a 10 μM concentration.

The results demonstrate than ADGN peptides significantly improve viral-mediated gene delivery in non-permissive cell lines, including human hepatocyte HepG2 and human neuronal HCN2 cells. ADGN peptides increased infectivity of all tested AAV serotypes including AAV-1. AAV-2. AAV-5, AAV-6, and AAV-8 at an MOI that resulted in less than 5% GFP-positive cells in the absence of peptides. ADGN peptides promoted viral gene delivery in a dose-dependent manner starting at 0.1 μM and exhibiting an optimal effect at 100 μM. Interestingly, for all ADGN peptides tested, more that 60% of the maximal increase was already observed at a CPP concentration of 10 μM.

In comparison to other known CPPs, including TAT-HA2 or LAH4 (SEQ ID NO: 84; which is a leucine- and histidine-rich peptide), ADGN-103a, ADGN-103b, and ADGN-109 were at least 4- to 6-fold more potent. Previous work (Lui and collaborators, Molecular Therapy, 2014) showed that using TAT-HA2 or LAH4 peptides improved AAV-2 efficiency for tranfection of HepG2 cells. Lui et al demonstrated that using a 200 μM concentration of TAT-HA2 or LAH4 peptides lead to about 25% and 45% of GFP-positive cells, respectively, which is 4- and 2-fold lower than the results obtained with the ADGN peptides. Interestingly, at a lower concentration of CPPs of 10 AM, only 12% GFP-positive cells were obtained using either TAT-HA2 or LAH4 by Liu et al. Given that at the same dose ADGN peptides led to 87% to 95% GFP-positive cells, the ADGN peptides are up to 7- to 8-fold more potent than TAT-HA2 or LAH4, and significantly lower concentrations of ADGN peptides are required to improve cellular uptake of viral particles.

### Example 3: Cell Penetrating Peptides (CPPs) Significantly Increase Virus Mediated Gene Delivery In Vivo

In the present example, the impact of cell penetrating peptides on AAV-2, AAV-6, and AAV-8 mediated gene delivery in an in vivo mouse model is investigated. The purpose of the example is the comparison of the invention CPPs (e.g., PEP-1, PEP-2, VEPEP-3a, VEPEP-3b, VEPEP-6, VEPEP-9, ADGN-100a, and ADGN-100b) with previously described CPPs, such as pANT and TAT-HA2, and to demonstrate that association with the invention CPPs results in significant enhancement of AAV2, AAV6, and AAV8-mediated transduction in vivo by either systemic or local intramuscular/subcutaneous injection. A major limitation of using AAV in the clinic is related to the fact that doses of AAV vector required to obtain significant transgenic expression induce strong immune responses. Therefore, the minimal dose required to produce a significant and detectable level of gene expression will be determined.

Adeno-associated viruses (e.g., AAV-2, AAV-6, and AAV-8) encoding a transgene, e.g., green fluorescent protein (AAV-GFP) or relevant therapeutic genes, such as for hemophilia A (AAV-Factor VIII), hemophilia B (AAV-Factor IX), biallelic RPE65-mediated inherited retinal disease (IRD) (AAV-RPE65), choroideremia (AAV-CHM), Leber hereditary optic neuropathy (LHON), TPP1 deficiency (AAV-TPP1), or retinitis pigmentosa, such as rhodopsin-linked autosomal dominant retinitis pigmentosa (adRP) (AAV-Rho), are produced in a permissive cell type, e.g., HEK 293 cells. Peptides are obtained by solid phase synthesis. Appropriate disease model mice (e.g., BALB/C mice and the like) are used for the administration of AAV and AAV/CPP complexes. AAV encoding green fluorescent protein (AAV2-GFP, AAV-6 GFP and AAV-8 GFP) and the therapeutic genes at 10⁷ to 10¹² particles/mouse (e.g., 1-4×10⁹ particles/mouse) are pre-incubated in physiological NaCl buffer with varying concentrations of CPPs, e.g., about 1, 10, and 100 μM, for 30 min. The peptide dose per mouse can vary, e.g., between 10 μg to 1000 μg.

AAV alone or CPP/AAV complexes in a 200 μl final volume are intravenously, intramuscularly or subcutaneously injected into BALB/C mice (4 mice per group). Viral mediated transgene expression is monitored 10 to 30 days after injection, to estimate the persistence of the expression. GFP expression or expression of the therapeutic genes in the different tissues is analyzed, e.g., by immunofluorescence, epifluorescence, or PCR. Therapeutic gene products can also be measured in the blood or plasma by standard techniques. The level of GFP or therapeutic gene products is compared to free AAV and non-injected mice groups for different tissues including, e.g., lung, liver, spleen, brain, heart, muscle and pancreas. The impact of the CPPs on the AAV in vivo distribution is determined. Data are reported, e.g., as an average of 3 mice per group.

The impact of the CPP on the toxicity and on AAV-associated immune response will be investigated. In all cases, histological study of the major organ including liver, spleen, lung and muscle will be performed. We previously demonstrated CPPs doesn't increase the level of inflammatory cytokine in vivo. The level of cytokine, interleukin and TNF will be evaluated at different time point post injection using a 20 plex cytokine test and compared to free AAV injection. Mice will be bled, e.g., at weeks 2, 4, 6, 8 and 12 after vector administration and transgene product levels as well as AAV neutralizing antibodies will be measured. In a repeat administration experiment, the concentration dose selected based on efficacy and toxicity results observed after Day 1 injection will be used in the second AAV administration at Day 30. Anti-AAV IgG and neutralizing antibody levels for each experimental group at selected time points after AAV administration will be determined.

The CPPs can significantly enhance AAV mediated gene delivery in vivo without affecting the host cell immune responses to AAV vectors. CPPs can enable efficient vector transduction at much lower doses of AAVs and potentially limit or avoid immune responses.

CPPs may increase the overall safety of AAV in gene delivery and allow multiple injection/doses by preventing the access of the AAV to the host immune system.

### Example 4: Optimization of Cell Penetrating Peptides (CPP)

### Peptide Chemistry

Peptides were obtained by solid phase synthesis. L peptides or retro Inverso Peptides (ADGN-100; ADGN-106, ADGN-109) can be modified either on N- or C-terminus with the following motifs.

VEPEP-3a:
βAKWFERWFREWPRKRR
VEPEP-3b:
βAKWWERWWREWPRKRR
VEPEP-6:
βALWRALWRLWRSLWRLLWKA
VEPEP-9:
βALRWWLRWASRWFSRWAWWR
ADGN-100a:
βAKWRSAGWRWRLWRVRSWSR
ADGN-100b:
βAKWRSALYRWRLWRVRSWSR

Specific targeting sequences GYVS (K) or YIGS (R) is added at the C or N-terminus of the peptide separated by a linker as reported below. Linkers correspond to either Gly-beta Ala or Gly4-Ser motif. As example the sequence of ADGN-100 is modified as following.

GYVSK-GGGGS-KWRSAGWRWRLWRVRSWSR
KWRSAGWRWRLWRVRSWSR-(S)GGGG-GYVSK
GYVSK-GβA-KWRSAGWRWRLWRVRSWSR

Mono-PEGylated-Peptide conjugation (peptide conjugated to 10 kDa PEG or 5 kDa PEG) was performed at the primary amino group of the N-terminal beta Alanine residues, using aldehyde monoethoxypoly (ethylene glycol) at pH 5.5, then PEGylated-peptide was further purified by RP-HPLC and analyzed by electrospray ionization mass spectroscopy).

Dopamine improved interaction with aromatic and hydrophobic patches at the surface of protein. Dopamine was added to the N-terminus of the peptide using a Gly4S linker or G-βAla.

### Particle Stability Evaluation

Stock solutions of peptides were prepared at 2 mg/mL in distilled water or 5% DMSO and sonicated for 10 min in a water bath sonicator then diluted just before use. AAV encoding green fluorescent protein at MOI of 300 was pre-incubated in low salt medium with CPPs containing either a specific targeting motif, a PEG or Dopamine moiety at concentration of 50 and 100 μM for 30 min. AAV/CPP particle stability was evaluated in different conditions. Particles were incubated for 1 hour in medium containing 10, 20 to 50% serum (FSC) and in the presence of heparin (5 and 10 μg). Then AAV alone or CPP/AAV complexes were added to cultured cells in a 24 well plate format. Cells were treated with either free AAV or CPP/AAV complexes for 4 h, and then the medium was replaced with fresh medium. Viral mediated transgene expression was monitored 2 days post-infection. GFP expressing cells were detected by flow cytometry.

### Example 5: Optimization of PEP-1 Peptides for Virus Delivery in 293 T Cells

### Peptide Synthesis and Chemistry

Pep-1 peptides were synthesized by solid-phase peptide synthesis according to Fmoc/tBoc method. All peptides were N-acetylated and bear a cysteamide group at their carboxy-terminus (—NH—CH2-CH2-SH). The crude peptide was purified by RP-HPLC on a C18 column (Interchrom UP5 WOD/25M Uptispere 300 5 ODB, 250_21.2 mm). Specific targeting sequence GYVSK was added at the C or N-terminus of the peptide separated by a Gly₄ or Gly₄-Ser linker as described below.

PEP-1
KETWWETWWTEWSQPKKKRKV
PEP-1 T1
GYVSK-GGGGSKETWWETWWTEWSQPKKKRKV
PEP-1 T2
KETWWETWWTEWSQPKKKRKVGGGG-GYVSK
5 KDa PEG-PEGylated PEP-1

Mono-PEGylated-Peptide conjugation (peptide conjugated with 5 kDa PEG) was performed at the primary amino group of the N-terminal beta Alanine residues, using aldehyde monoethoxypoly (ethylene glycol) at pH 5.5, then PEGylated-peptide was further purified by RP-HPLC and analyzed by electrospray ionization mass spectroscopy).

PEP-1-PEG:
PEG-βAKETWWETWWTEWSQPKKKRKV
PEP-1-DOPA
DOPA-GGGG-KETWWETWWTEWSQPKKKRKV
(Dopamine was added to the N-terminus of the
peptide using a Gly4 linker)

### Particle Stability Evaluation.

Stock solutions of peptides were prepared at 2 mg/mL in distilled water or 5% DMSO and sonicated for 10 min in a water bath sonicator then diluted just before use. AAV-2 encoding green fluorescent protein at MOI of 300 was pre-incubated in low salt medium with PEP-1 peptides containing either a specific targeting motif, a PEG or Dopamine moiety at a concentration of 50 μM for 30 min. The AAV-2/peptide particle stability was evaluated in different conditions. Particles were incubated for 1 hour in DMEM medium containing 10, 20 to 50% serum (FSC) or in the presence of heparin (5 and 10 μg). Then AAV alone or PEP-1/AAV complexes were added to cultured 293 T cells in a 24 well plate format. Cells were treated with either free AAV-2 or Peptide/AAV-2 complexes for 4 h, and then the medium was replaced with fresh medium. Viral mediated transgene expression was monitored 2 days post-infection. GFP expressing cells were detected by flow cytometry.

### Results

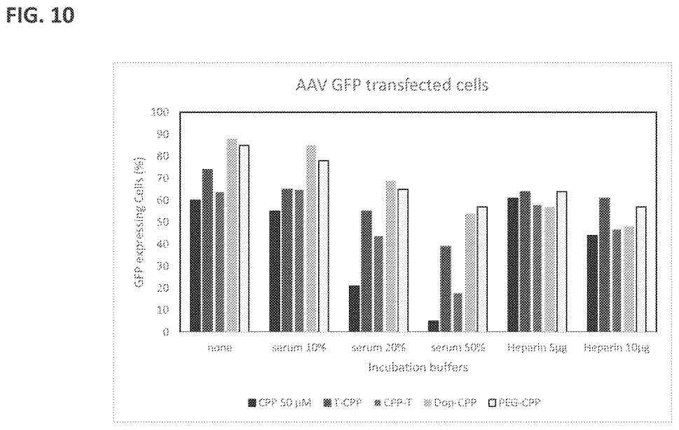

As reported in the FIG. 10 below, in the absence of any modification, the presence of 20% and 50% serum significantly reduced stability of AAV/PEP-1 complexes. The level of GFP expressing cells is reduced by 60% and 80%, respectively. In contrast, the fact that the incubation in the presence of Heparin had only a limited effect on the stability suggested that interactions between PEP-1 and AAV do not involved electrostatic contact and are mainly hydrophobic.

Dopa and PEG modifications of PEP-1 increased efficacy by 40% in standard conditions (no serum, no heparin) and stabilized Peptide/AAV-2 particles in the presence of high concentration of scrum. Efficacy is reduced by only 30% in 50% serum.

The presence of GYVSK targeting sequence at the C-terminus of PEP-1 had only a moderate effect on the AAV/PEP-1 complex stability. In contrast, the presence of the targeting sequence to the N-terminus significantly protected the complex from serum. Efficacy was reduced by only 27% in 50% serum conditions.

Sequence Listing

| SEQ ID | Sequence | Annotations |
|---|---|---|
| 1 | X₁X₂X₃X₄X₅X₂X₃X₄X₆X₇X₃X₈X₉X₁₀X₁₁X₁₂X₁₃ | VEPEP-3 |
| | X₁ is beta-A or S, X₂ is K, R or L, X₃ is F or W, X₄ is F, | |
| | W or Y, X₅ is E, R or S, X₆ is R, T or S, X₇ is E, R, or | |
| | S, X₈ is none, F or W, X₉ is P or R, X₁₀ is R or L, X₁₁ is | |
| | K, W or R, X₁₂ is R or F, and X₁₃ is R or K | |
| 2 | X₁X₂WX₄EX₂WX₄X₆X₇X₃PRX₁₁RX₁₃ | VEPEP-3 1 |
| | X₁ is beta-A or S, X₂ is R or K, X₃ is W or F, | |
| | X₄ is F, W, or Y, X₆ is T or R, X₇ is E or R, | |
| | X₁₁ is R or K, and X₁₃ is R or K | |
| 3 | X₁KWFERWFREWPRKRR | VEPEP-3 1a |
| | X₁ is beta-A or S | |
| 4 | X₁KWWERWWREWPRKRR | VEPEP-3 1b |
| | X₁ is beta-A or S | |
| 5 | X₁KWWERWWREWPRKRK | VEPEP-3 1c |
| | X₁ is beta-A or S | |
| 6 | X₁RWWEKWWTRWPRKRK | VEPEP-3 1d |
| | X₁ is beta-A or S | |
| 7 | X₁RWYEKWYTEFPRRRR | VEPEP-3 1e |
| | X₁ is beta-A or S | |
| 8 | X₁KX₁₄WWERWWRX₁₄WPRKRK | VEPEP-3 1S |
| | X₁ is beta-A or S and X₁₄ is a non-natural | |
| | amino acid, and wherein there is a | |
| | hydrocarbon linkage between the two | |
| | non-natural amino acids | |
| 9 | X₁X₂X₃WX₅X₁₀X₃WX₆X₇WX₈X₉X₁₀WX₁₂R | VEPEP-3 2 |
| | X₁ is beta-A or S, X₂ is K, R or L, X₃ is F or | |
| | W, X₅ is R or S, X₆ is R or S, X₇ is R or S, X₈ | |
| | is F or W, X₉ is R or P, X₁₀ is L or R, and | |
| | X₁₂ is R or F | |
| 10 | X₁RWWRLWWRSWFRLWRR | VEPEP-3 2a |
| | X₁ is beta-A or S | |
| 11 | X₁LWWRRWWSRWWPRWRR | VEPEP-3 2b |
| | X₁ is beta-A or S | |
| 12 | X₁LWWSRWWRSWFRLWFR | VEPEP-3 2c |
| | X₁ is beta-A or S | |
| 13 | X₁KFWSRFWRSWFRLWRR | VEPEP-3 2d |
| | X₁ is beta-A or S | |
| 14 | X₁RWWX₁₄LWWRSWX₁₄RLWRR | VEPEP-3 2S |
| | X₁ is a beta-alanine or a serine and X₁₄ is | |
| | a non-natural amino acid, and wherein | |
| | there is a hydrocarbon linkage between | |
| | the two non-natural amino acids | |
| 15 | X₁LX₂RALWX₉LX₃X₉X₄LWX₉LX₅X₆X₇X₈ | VEPEP-6 1 |
| | X₁ is beta-A or S, X₂ is F or W, X₃ is L, W, | |
| | C or I, X₄ is S, A, N or T, X₅ is L or W, X₆ is | |
| | W or R, X₇ is K or R, X₈ is A or none, and | |
| | X₉ is R or S | |
| 16 | X₁LX₂LARWX₉LX₃X₉X₄LWX₉LX₅X₆X₇X₈ | VEPEP-6 2 |
| | X₁ is beta-A or S, X₂ is F or W, X₃ is L, W, | |
| | C or I, X₄ is S, A, N or T, X₅ is L or W, X₆ is | |
| | W or R, X₇ is K or R, X₈ is A or none, and | |
| | X₉ is R or S | |
| 17 | X₁LX₂ARLWX₉LX₃X₉X₄LWX₉LX₅X₆X₇X₈ | VEPEP-6 3 |
| | X₁ is beta-A or S, X₂ is F or W, X₃ is L, W, | |
| | C or I, X₄ is S, A, N or T, X₅ is L or W, X₆ is | |
| | W or R, X₇ is K or R, X₈ is A or none, and | |
| | X₉ is R or S | |
| 18 | X₁LX₂RALWRLX₃RX₄LWRLX₅X₆X₇X₈ | VEPEP-6 4 |
| | X₁ is beta-A or S, X₂ is F or W, X₃ is L, W, | |
| | C or I, X₄ is S, A, N or T, X₅ is L or W, X₆ is | |
| | W or R, X₇ is K or R, and X₈ is A or none | |
| 19 | X₁LX₂RALWRLX₃RX₄LWRLX₅X₆KX₇ | VEPEP-6 5 |
| | X₁ is beta-A or S, X₂ is F or W, X₃ is L or | |
| | W, X₄ is S, A or N, X₅ is L or W, X₆ is W or | |
| | R, X₇ is A or none | |
| 20 | X₁LFRALWRLLRX₂LWRLLWX₃ | VEPEP-6 6 |
| | X₁ is beta-A or S, X₂ is S or T, and X₃ is K or | |
| | R | |
| 21 | X₁LWRALWRLWRX₂LWRLLWX₃A | VEPEP-6 7 |
| | X₁ is beta-A or S, X₂ is S or T, and X₃ is K | |
| | or R | |
| 22 | X₁LWRALWRLX₄RX₂LWRLWRX₃A | VEPEP-6 8 |
| | X₁ is beta-A or S, X₂ is S or T, X₃ is K or R, | |
| | and X₄ is L, C or I | |
| 23 | X₁LWRALWRLWRX₂LWRLWRX₃A | VEPEP-6 9 |
| | X1 is beta-A or S, X2 is S or T, and X3 is K | |
| | or R | |
| 24 | X₁LWRALWRLX₅RALWRLLWX₃A | VEPEP-6 10 |
| | X₁ is beta-A or S, X₃ is K or R, and X₅ is L | |
| | or I | |
| 25 | X₁LWRALWRLX₄RNLWRLLWX₃A | VEPEP-6 11 |
| | X₁ is beta-A or S, X₃ is K or R, and X₄ is L, | |
| | C or I | |
| 26 | Ac-X₁LFRALWRLLRSLWRLLWK- | VEPEP-6a |
| | cysteamide | |
| | X₁ is beta-A or S | |
| 27 | Ac-X₁LWRALWRLWRSLWRLLWKA- | VEPEP-6b |
| | cysteamide | |
| | X₁ is beta-A or S | |
| 28 | Ac-X₁LWRALWRLLRSLWRLWRKA- | VEPEP-6c |
| | cysteamide | |
| | X₁ is beta-A or S | |
| 29 | Ac-X₁LWRALWRLWRSLWRLWRKA- | VEPEP-6d |
| | cysteamide | |
| | X₁ is beta-A or S | |
| 30 | Ac-X₁LWRALWRLLRALWRLLWKA- | VEPEP-6e |
| | cysteamide | |
| | X₁ is beta-A or S | |
| 31 | Ac-X₁LWRALWRLLRNLWRLLWKA- | VEPEP-6f |
| | cysteamide | |
| | X₁ is beta-A or S | |
| 32 | Ac-X₁LFRALWRₛLLRSₛLWRLLWK- | ST-VEPEP-6a |
| | cysteamide | |
| | X₁ is beta-A or S and the residues | |
| | followed by an inferior ″s″ are linked by | |
| | a hydrocarbon linkage | |
| 33 | Ac-X₁LFLARWRₛLLRSₛLWRLLWK- | ST-VEPEP-6aa |
| | cysteamide | |
| | X₁ is beta-A or S and the residues | |
| | followed by an inferior ″s″ are linked by | |
| | a hydrocarbon linkage | |
| 34 | Ac-X₁LFRALWSₛLLRSₛLWRLLWK- | ST-VEPEP-6ab |
| | cysteamide | |
| | X₁ is beta-A or S and the residues | |
| | followed by an inferior ″s″ are linked by | |
| | a hydrocarbon linkage | |
| 35 | Ac-X₁LFLARWSₛLLRSₛLWRLLWK- | ST-VEPEP-6ad |
| | cysteamide | |
| | X₁ is beta-A or S and the residues | |
| | followed by an inferior ″s″ are linekd by | |
| | a hydrocarbon linkage | |
| 36 | Ac-X₁LFRALWRLLRₛSLWSₛLLWK- | ST-VEPEP-6b |
| | X₁ is beta-A or S and the residues | |
| | followed by an inferior ″s″ are linked by | |
| | a hydrocarbon linkage | |
| 37 | Ac-X₁LFLARWRLLRₛSLWSₛLLWK- | ST-VEPEP-6ba |
| | cysteamide | |
| | X₁ is beta-A or S and the residues | |
| | followed by an inferior ″s″ are linked by | |
| | a hydrcarbon linkage | |
| 38 | Ac-X₁LFRALWRLLSₛSLWSₛLLWK- | ST-VEPEP-6bb |
| | cysteamide | |
| | X₁ is beta-A or S and the residues | |
| | followed by an inferior ″s″ are linked by | |
| | a hydrocarbon linkage | |
| 39 | Ac-X₁LFLARWRLLSₛSLWSₛLLWK- | ST-VEPEP-6bd |
| | cysteamide | |
| | X₁ is beta-A or S and the residues | |
| | followed by an inferior ″s″ are linked by | |
| | a hydrocarbon linkage | |
| 40 | Ac-X₁LFARₛLWRLLRSₛLWRLLWK- | ST-VEPEP-6c |
| | cysteamide | |
| | X₁ is beta-A or S and the residues | |
| | followed by an inferior ″s″ are linked by | |
| | a hydrocarbon linkage | |
| 41 | X₁X₂X₃WWX₄X₅WAX₆X₃X₇X₈X₉X₁₀X₁₁X₁₂WX₁₃R | VEPEP-9 1 |
| | X₁ is beta-A or S, X₂ is L or none, X₃ is R | |
| | or none X, X₄ is L, R or G, X₅ is R, W or S, X₆ | |
| | is S, P or T, X₇ is W or P, X₈ is F, A or R, X₉ | |
| | is S, L, P or R, X₁₀ is R or S, X₁₁ is W or | |
| | none, X₁₂ is A, R or none and X₁₃ is W or | |
| | F, and wherein if X₃ is none, then X₂, X₁₁ | |
| | and X₁₂ are none as well | |
| 42 | X₁X₂RWWLRWAX₆RWX₈X₉X₁₀WX₁₂WX₁₃R | VEPEP-9 2 |
| | X₁ is beta-A or S, X₂ is L or none, X₆ is S or | |
| | P, X₈ is F or A, X₉ is S, L or P, X₁₀ is R or S, | |
| | X₁₂ is A or R, and X₁₃ is W or F | |
| 43 | X₁LRWWLRWASRWFSRWAWWR | VEPEP9a1 |
| | X₁ is beta-A or S | |
| 44 | X₁LRWWLRWASRWASRWAWFR | VEPEP9a2 |
| | X₁ is beta-A or S | |
| 45 | X₁RWWLRWASRWALSWRWWR | VEPEP9b1 |
| | X₁ is beta-A or S | |
| 46 | X₁RWWLRWASRWFLSWRWWR | VEPEP9b2 |
| | X₁ is beta-A or S | |
| 47 | X₁RWWLRWAPRWFPSWRWWR | VEPEP9c1 |
| | X₁ is beta-A or S | |
| 48 | X₁RWWLRWASRWAPSWRWWR | VEPEP9c2 |
| | X₁ is beta-A or S | |
| 49 | X₁WWX₄X₅WAX₆X₇X₈RX₁₀WWR | VEPEP-9 3 |
| | X₁ is beta-A or S | |
| 50 | X₁WWRWWASWARSWWR | VEPEP9d |
| | X₁ is beta-A or S | |
| 51 | X₁WWGSWATPRRRWWR | VEPEP9e |
| | X₁ is beta-A or S | |
| 52 | X₁WWRWWAPWARSWWR | VEPEP9f |
| | X₁ is beta-A or S | |
| 53 | X₁KWRSX₂X₃X₄RWRLWRX₅X₆X₇X₈SR | ADGN-100 |
| | X₁ is any amino acid or none, and X₂-X₈ | |
| | are any amino acid | |
| 54 | X₁KWRSX₂X₃X₄RWRLWRX₅X₆X₇X₈SR | ADGN-100 1 |
| | X₁ IS βA, S, or none, X₂ is A or V, X₃ is G or | |
| | L, X₄ is W or Y, X₅ is V or S, X₆ is R, V, or A, | |
| | X₇ is S or L, and X₈ is W or Y | |
| 55 | KWRSAGWRWRLWRVRSWSR | ADGN-100a |
| 56 | KWRSALYRWRLWRVRSWSR | ADGN-100b |
| 57 | KWRSALYRWRLWRSRSWSR | ADGN-100c |
| 58 | KWRSALYRWRLWRSALYSR | ADGN-100d |
| 59 | KWRS_{S}AGWR_{S}WRLWRVRSWSR | ADGN-100 aa |
| | the residues marked with a subscript ″S″ | |
| | are linked by a hydrocarbon linkage | |
| 60 | KWR_{S}SAGWRWR_{S}LWRVRSWSR | ADGN-100 ab |
| | the residues marked with a subscript ″S″ | |
| | are linked by a hydrocarbon linkage | |
| 61 | KWRSAGWR_{S}WRLWRVR_{S}SWSR | ADGN-100 ac |
| | the residues marked with a subscript ″S″ | |
| | are linked by a hydrocarbon linkage | |
| 62 | KWRS_{S}ALYR_{S}WRLWRSRSWSR | ADGN-100 ba |
| | the residues marked with a subscript ″S″ | |
| | are linked by a hydrocarbon linkage | |
| 63 | KWR_{S}SALYRWR_{S}LWRSRSWSR | ADGN-100 bb |
| | the residues marked with a subscript ″S″ | |
| | are linked by a hydrocarbon linkage | |
| 64 | KWRSALYR_{S}WRLWRSR_{S}SWSR | ADGN-100 bc |
| | the residues marked with a subscript ″S″ | |
| | are linked by a hydrocarbon linkage | |
| 65 | KWRSALYRWR_{S}LWRS_{S}RSWSR | ADGN-100 bd |
| | the residues marked with a subscript ″S″ | |
| | are linked by a hydrocarbon linkage | |
| 66 | KWRSALYRWRLWRS_{S}RSWS_{S}R | ADGN-100 be |
| | the residues marked with a subscript ″S″ | |
| | are linked by a hydrocarbon linkage | |
| 67 | KWR_{S}SALYRWR_{S}LWRSALYSR | ADGN-100 ca |
| | the residues marked with a subscript ″S″ | |
| | are linked by a hydrocarbon linkage | |
| 68 | KWRS_{S}ALYR_{S}WRLWRSALYSR | ADGN-100 cb |
| | the residues marked with a subscript ″S″ | |
| | are linked by a hydrocarbon linkage | |
| 69 | KWRSALYRWR_{S}LWRS_{S}ALYSR | ADGN-100 cc |
| | the residues marked with a subscript ″S″ | |
| | are linked by a hydrocarbon linkage | |
| 70 | KWRSALYRWRLWRS_{S}ALYS_{S}R | ADGN-100 cd |
| | the residues marked with a subscript ″S″ | |
| | are linked by a hydrocarbon linkage | |
| 71 | KETWWETWWTEWSQPKKKRKV | PEP-1 |
| 72 | KETWFETWFTEWSQPKKKRKV | PEP-2 |
| 73 | KWFETWFTEWPKKRK | PEP-3 |
| 74 | GALFLGFLGAAGSTMGAWSQPKKKRKV | MPG |
| 75 | beta-AKWFERWFREWPRKRR | VEPEP-3a |
| 76 | beta-AKWWERWWREWPRKRR | VEPEP-3b |
| 77 | beta-ALWRALWRLWRSLWRLLWKA | VEPEP-6 |
| 78 | beta-ALRWWLRWASRWFSRWAWWR | VEPEP-9 |
| 79 | beta-AKWRSAGWRWRLWRVRSWSR | ADGN-100a |
| 80 | beta-AKWRSALYRWRLWRVRSWSR | ADGN-100b |
| 81 | GLWRALWRLLRSLWRLLWKV | CADY |
| 82 | RQIKIWFQNRRMKWKKC | pANT |
| 83 | CRRRQRRKKRGGDIMGEWGNEIFGAIAGFLG | TAT-HA2 |
| 84 | KKALLALALHHLAHLALHLALALKKAC | LAH4 |

1: A virus delivery complex for intracellular delivery of a virus comprising a cell-penetrating peptide and the virus, wherein the cell-penetrating peptide is selected from the group consisting of PEP-1 peptides, PEP-2 peptides, PEP-3 peptides, VEPEP-3 peptides, VEPEP-6 peptides, VEPEP-9 peptides, and ADGN-100 peptides.

2: The virus delivery complex of claim 1, wherein the cell-penetrating peptide is a VEPEP-3 peptide.

3-4. (canceled)

5: The virus delivery complex of claim 1, wherein the cell-penetrating peptide is a VEPEP-6 peptide.

6-7. (canceled)

8: The virus delivery complex of claim 1, wherein the cell-penetrating peptide is a VEPEP-9 peptide.

9-10. (canceled)

11: The virus delivery complex of claim 1, wherein the cell-penetrating peptide is an ADGN-100 peptide.

12-15. (canceled)

16: The virus delivery complex of claim 1, wherein the cell-penetrating peptide is covalently linked to the virus.

17. (canceled)

18: The virus delivery complex of claim 1, wherein the cell-penetrating peptide further comprises a cysteamide moiety covalently linked to its C-terminus.

19: The virus delivery complex of claim 1, wherein at least some of the cell-penetrating peptides in the virus delivery complex are linked to a targeting moiety by a linkage.

20: The virus delivery complex of claim 1, wherein the virus is a recombinant virus.

21: The virus delivery complex of claim 20, wherein the recombinant virus is recombinant adeno-associated virus (AAV), adenovirus, lentivirus, retrovirus, herpes simplex virus (HSV), poxvirus, Epstein-Barr virus (EBV), vaccinia virus, or human cytomegalovirus (hCMV).

22. (canceled)

23: The virus delivery complex of claim 1, wherein the average diameter of the virus delivery complex is between about 20 nm and about 1000 nm.

24: A nanoparticle comprising a core comprising the virus delivery complex of claim 1.

25: The nanoparticle of claim 24, wherein the core further comprises one or more additional virus delivery complexes according to claim 1.

26: The nanoparticle of claim 24, wherein at least some of the cell-penetrating peptides in the nanoparticle are linked to a targeting moiety by a linkage.

27: The nanoparticle of claim 24, wherein the core is coated by a shell comprising a peripheral cell-penetrating peptide.

28: A pharmaceutical composition comprising the virus delivery complex of claim 1, and a pharmaceutically acceptable carrier.

29: A method of preparing the virus delivery complex of claim 1, comprising combining the cell-penetrating peptide with the one or more viruses, thereby forming the virus delivery complex.

30: A method of delivering one or more viruses into a cell, comprising contacting the cell with the virus delivery complex of claim 1, wherein the virus delivery complex or the nanoparticle comprises the one or more viruses.

31-32. (canceled)

33: A method of treating a disease in an individual comprising administering to the individual an effective amount of the pharmaceutical composition of claim 28.

34-40. (canceled)

41: A kit comprising a composition comprising the virus delivery complex of claim 1.


SEQUENCE LISTING (99 sequences)

SEQ ID NO: 1 - protein, 17 aa, Artificial Sequence
  Synthetic Construct
  VARIANT 1: Xaa = beta-A or S
  VARIANT 2, 6: Xaa = K, R or L
  VARIANT 3, 7, 11: Xaa = F or W
  VARIANT 4, 8: Xaa = F, W or Y
  VARIANT 5: Xaa = E, R or S
  VARIANT 9: Xaa = R, T or S
  VARIANT 10: Xaa = E, R, or S
  VARIANT 12: Xaa = none, F or W
  VARIANT 13: Xaa = P or R
  VARIANT 14: Xaa = R or L
  VARIANT 15: Xaa = K, W or R
  VARIANT 16: Xaa = R or F
  VARIANT 17: Xaa = R or K
XXXXXXXXXXXXXXXXX

SEQ ID NO: 2 - protein, 16 aa, Artificial Sequence
  Synthetic Construct
  VARIANT 1: Xaa = beta-A or S
  VARIANT 2, 6: Xaa = K, R or L
  VARIANT 4, 8: Xaa = F, W or Y
  VARIANT 9: Xaa = R, T or S
  VARIANT 10: Xaa = E, R, or S
  VARIANT 11: Xaa = F or W
  VARIANT 14: Xaa = K, W or R
  VARIANT 16: Xaa = R or K
XXWXEXWXXXXPRXRX

SEQ ID NO: 3 - protein, 16 aa, Artificial Sequence
  Synthetic Construct
  VARIANT 1: Xaa = beta-A or S
XKWFERWFREWPRKRR

SEQ ID NO: 4 - protein, 16 aa, Artificial Sequence
  Synthetic Construct
  VARIANT 1: Xaa = beta-A or S
XKWWERWWREWPRKRR

SEQ ID NO: 5 - protein, 16 aa, Artificial Sequence
  Synthetic Construct
  VARIANT 1: Xaa = beta-A or S
XKWWERWWREWPRKRK

SEQ ID NO: 6 - protein, 16 aa, Artificial Sequence
  Synthetic Construct
  VARIANT 1: Xaa = beta-A or S
XRWWEKWWTRWPRKRK

SEQ ID NO: 7 - protein, 16 aa, Artificial Sequence
  Synthetic Construct
  VARIANT 1: Xaa = beta-A or S
XRWYEKWYTEFPRRRR

SEQ ID NO: 8 - protein, 17 aa, Artificial Sequence
  Synthetic Construct
  VARIANT 1: Xaa = beta-A or S
  VARIANT 3, 11: Xaa = Any non-natural amino acid
XKXWWERWWRXWPRKRK

SEQ ID NO: 9 - protein, 17 aa, Artificial Sequence
  Synthetic Construct
  VARIANT 1: Xaa = beta-A or S
  VARIANT 2: Xaa = K, R or L
  VARIANT 3, 7: Xaa = F or W
  VARIANT 5: Xaa = R or S
  VARIANT 6, 14: Xaa =  L or R
  VARIANT 9: Xaa = R or S
  VARIANT 10: Xaa = R or S
  VARIANT 12: Xaa = F or W
  VARIANT 13: Xaa = R or P
  VARIANT 16: Xaa = R or F
XXXWXXXWXXWXXXWXR

SEQ ID NO: 10 - protein, 17 aa, Artificial Sequence
  Synthetic Construct
  VARIANT 1: Xaa = beta-A or S
XRWWRLWWRSWFRLWRR

SEQ ID NO: 11 - protein, 17 aa, Artificial Sequence
  Synthetic Construct
  VARIANT 1: Xaa = beta-A or S
XLWWRRWWSRWWPRWRR

SEQ ID NO: 12 - protein, 17 aa, Artificial Sequence
  Synthetic Construct
  VARIANT 1: Xaa = beta-A or S
XLWWSRWWRSWFRLWFR

SEQ ID NO: 13 - protein, 17 aa, Artificial Sequence
  Synthetic Construct
  VARIANT 1: Xaa = beta-A or S
XKFWSRFWRSWFRLWRR

SEQ ID NO: 14 - protein, 17 aa, Artificial Sequence
  Synthetic Construct
  VARIANT 1: Xaa = beta-A or S
  VARIANT 5, 12: Xaa = Any non-natural amino acid
XRWWXLWWRSWXRLWRR

SEQ ID NO: 15 - protein, 20 aa, Artificial Sequence
  Synthetic Construct
  VARIANT 1: Xaa = beta-A or S
  VARIANT 3: Xaa = F or W
  VARIANT 8, 11, 15: Xaa = R or S
  VARIANT 10: Xaa = L, W, C or I
  VARIANT 12: Xaa = S, A, N or T
  VARIANT 17: Xaa = L or W
  VARIANT 18: Xaa = W or R
  VARIANT 19: Xaa = K or R
  VARIANT 20: Xaa = A or none
XLXRALWXLXXXLWXLXXXX

SEQ ID NO: 16 - protein, 20 aa, Artificial Sequence
  Synthetic Construct
  VARIANT 1: Xaa = beta-A or S
  VARIANT 3: Xaa = F or W
  VARIANT 8, 11, 15: Xaa = R or S
  VARIANT 10: Xaa = L, W, C or I
  VARIANT 12: Xaa = S, A, N or T
  VARIANT 17: Xaa = L or W
  VARIANT 18: Xaa = W or R
  VARIANT 19: Xaa = K or R
  VARIANT 20: Xaa = A or none
XLXLARWXLXXXLWXLXXXX

SEQ ID NO: 17 - protein, 20 aa, Artificial Sequence
  Synthetic Construct
  VARIANT 1: Xaa = beta-A or S
  VARIANT 3: Xaa = F or W
  VARIANT 8, 11, 15: Xaa = R or S
  VARIANT 10: Xaa = L, W, C or I
  VARIANT 12: Xaa = S, A, N or T
  VARIANT 17: Xaa = L or W
  VARIANT 18: Xaa = W or R
  VARIANT 19: Xaa = K or R
  VARIANT 20: Xaa = A or none
XLXARLWXLXXXLWXLXXXX

SEQ ID NO: 18 - protein, 20 aa, Artificial Sequence
  Synthetic Construct
  VARIANT 1: Xaa = beta-A or S
  VARIANT 3: Xaa = F or W
  VARIANT 10: Xaa = L, W, C or I
  VARIANT 12: Xaa = S, A, N or T
  VARIANT 17: Xaa = L or W
  VARIANT 18: Xaa = W or R
  VARIANT 19: Xaa = K or R
  VARIANT 20: Xaa = A or none
XLXRALWRLXRXLWRLXXXX

SEQ ID NO: 19 - protein, 20 aa, Artificial Sequence
  Synthetic Construct
  VARIANT 1: Xaa = beta-A or S
  VARIANT 3: Xaa = F or W
  VARIANT 10: Xaa = L or W
  VARIANT 12: Xaa = S, A or N
  VARIANT 17: Xaa =  L or W
  VARIANT 18: Xaa = W or R
  VARIANT 20: Xaa = A or none
XLXRALWRLXRXLWRLXXKX

SEQ ID NO: 20 - protein, 19 aa, Artificial Sequence
  Synthetic Construct
  VARIANT 1: Xaa = beta-A or S
  VARIANT 12: Xaa = S or T
  VARIANT 19: Xaa = K or R
XLFRALWRLLRXLWRLLWX

SEQ ID NO: 21 - protein, 20 aa, Artificial Sequence
  Synthetic Construct
  VARIANT 1: Xaa = beta-A or S
  VARIANT 12: Xaa = S or T
  VARIANT 19: Xaa = K or R
XLWRALWRLWRXLWRLLWXA

SEQ ID NO: 22 - protein, 20 aa, Artificial Sequence
  Synthetic Construct
  VARIANT 1: Xaa = beta-A or S
  VARIANT 10: Xaa =  L, C or I
  VARIANT 12: Xaa = S or T
  VARIANT 19: Xaa = K or R
XLWRALWRLXRXLWRLWRXA

SEQ ID NO: 23 - protein, 20 aa, Artificial Sequence
  Synthetic Construct
  VARIANT 1: Xaa = beta-A or S
  VARIANT 12: Xaa = S or T
  VARIANT 19: Xaa = K or R
XLWRALWRLWRXLWRLWRXA

SEQ ID NO: 24 - protein, 20 aa, Artificial Sequence
  Synthetic Construct
  VARIANT 1: Xaa = beta-A or S
  VARIANT 10: Xaa = L or I
  VARIANT 19: Xaa = K or R
XLWRALWRLXRALWRLLWXA

SEQ ID NO: 25 - protein, 20 aa, Artificial Sequence
  Synthetic Construct
  VARIANT 1: Xaa = beta-A or S
  VARIANT 10: Xaa = L, C or I
  VARIANT 19: Xaa = K or R
XLWRALWRLXRNLWRLLWXA

SEQ ID NO: 26 - protein, 19 aa, Artificial Sequence
  Synthetic Construct
  VARIANT 1: Xaa = beta-A or S
XLFRALWRLLRSLWRLLWK

SEQ ID NO: 27 - protein, 20 aa, Artificial Sequence
  Synthetic Construct
  VARIANT 1: Xaa = beta-A or S
XLWRALWRLWRSLWRLLWKA

SEQ ID NO: 28 - protein, 20 aa, Artificial Sequence
  Synthetic Construct
  VARIANT 1: Xaa = beta-A or S
XLWRALWRLLRSLWRLWRKA

SEQ ID NO: 29 - protein, 20 aa, Artificial Sequence
  Synthetic Construct
  VARIANT 1: Xaa = beta-A or S
XLWRALWRLWRSLWRLWRKA

SEQ ID NO: 30 - protein, 20 aa, Artificial Sequence
  Synthetic Construct
  VARIANT 1: Xaa = beta-A or S
XLWRALWRLLRALWRLLWKA

SEQ ID NO: 31 - protein, 20 aa, Artificial Sequence
  Synthetic Construct
  VARIANT 1: Xaa = beta-A or S
XLWRALWRLLRNLWRLLWKA

SEQ ID NO: 32 - protein, 19 aa, Artificial Sequence
  Synthetic Construct
  VARIANT 1: Xaa = beta-A or S
XLFRALWRLLRSLWRLLWK

SEQ ID NO: 33 - protein, 19 aa, Artificial Sequence
  Synthetic Construct
  VARIANT 1: Xaa = beta-A or S
XLFLARWRLLRSLWRLLWK

SEQ ID NO: 34 - protein, 19 aa, Artificial Sequence
  Synthetic Construct
  VARIANT 1: Xaa = beta-A or S
XLFRALWSLLRSLWRLLWK

SEQ ID NO: 35 - protein, 19 aa, Artificial Sequence
  Synthetic Construct
  VARIANT 1: Xaa = beta-A or S
XLFLARWSLLRSLWRLLWK

SEQ ID NO: 36 - protein, 19 aa, Artificial Sequence
  Synthetic Construct
  VARIANT 1: Xaa = beta-A or S
XLFRALWRLLRSLWSLLWK

SEQ ID NO: 37 - protein, 19 aa, Artificial Sequence
  Synthetic Construct
  VARIANT 1: Xaa = beta-A or S
XLFLARWRLLRSLWSLLWK

SEQ ID NO: 38 - protein, 19 aa, Artificial Sequence
  Synthetic Construct
  VARIANT 1: Xaa = beta-A or S
XLFRALWRLLSSLWSLLWK

SEQ ID NO: 39 - protein, 19 aa, Artificial Sequence
  Synthetic Construct
  VARIANT 1: Xaa = beta-A or S
XLFLARWRLLSSLWSLLWK

SEQ ID NO: 40 - protein, 19 aa, Artificial Sequence
  Synthetic Construct
  VARIANT 1: Xaa = beta-A or S
XLFARLWRLLRSLWRLLWK

SEQ ID NO: 41 - protein, 20 aa, Artificial Sequence
  Synthetic Construct
  VARIANT 1: Xaa = beta-A or S
  VARIANT 2: Xaa = L or none if positions 3 and 11 are absent
  VARIANT 3, 11: Xaa = R or none
  VARIANT 6: Xaa = L, R or G
  VARIANT 7: Xaa = R, W or S
  VARIANT 10: Xaa = S, P or T
  VARIANT 12: Xaa = W or P
  VARIANT 13: Xaa = F, A or R
  VARIANT 14: Xaa = S, L, P or R
  VARIANT 15: Xaa = R or S
  VARIANT 16: Xaa = W or none if positions 3 and 11 are absent
  VARIANT 17: Xaa = A, R or none if positions 3 and 11 are absent
  VARIANT 19: Xaa = W or F
XXXWWXXWAXXXXXXXXWXR

SEQ ID NO: 42 - protein, 20 aa, Artificial Sequence
  Synthetic Construct
  VARIANT 1: Xaa = beta-A or S
  VARIANT 2: Xaa = L or none
  VARIANT 10: Xaa = S or P
  VARIANT 13: Xaa = F or A
  VARIANT 14: Xaa = S, L or P
  VARIANT 15: Xaa = R or S
  VARIANT 17: Xaa = A or R
  VARIANT 19: Xaa = W or F
XXRWWLRWAXRWXXXWXWXR

SEQ ID NO: 43 - protein, 20 aa, Artificial Sequence
  Synthetic Construct
  VARIANT 1: Xaa = beta-A or S
XLRWWLRWASRWFSRWAWWR

SEQ ID NO: 44 - protein, 20 aa, Artificial Sequence
  Synthetic Construct
  VARIANT 1: Xaa = beta-A or S
XLRWWLRWASRWASRWAWFR

SEQ ID NO: 45 - protein, 19 aa, Artificial Sequence
  Synthetic Construct
  VARIANT 1: Xaa = beta-A or S
XRWWLRWASRWALSWRWWR

SEQ ID NO: 46 - protein, 19 aa, Artificial Sequence
  Synthetic Construct
  VARIANT 1: Xaa = beta-A or S
XRWWLRWASRWFLSWRWWR

SEQ ID NO: 47 - protein, 19 aa, Artificial Sequence
  Synthetic Construct
  VARIANT 1: Xaa = beta-A or S
XRWWLRWAPRWFPSWRWWR

SEQ ID NO: 48 - protein, 19 aa, Artificial Sequence
  Synthetic Construct
  VARIANT 1: Xaa = beta-A or S
XRWWLRWASRWAPSWRWWR

SEQ ID NO: 49 - protein, 15 aa, Artificial Sequence
  Synthetic Construct
  VARIANT 1: Xaa = beta-A or S
  VARIANT 4: Xaa = R or G
  VARIANT 5: Xaa = W or S
  VARIANT 8: Xaa = S, T or P
  VARIANT 9: Xaa = W or P
  VARIANT 10: Xaa = A or R
  VARIANT 12: Xaa = S or R
XWWXXWAXXXRXWWR

SEQ ID NO: 50 - protein, 15 aa, Artificial Sequence
  Synthetic Construct
  VARIANT 1: Xaa = beta-A or S
XWWRWWASWARSWWR

SEQ ID NO: 51 - protein, 15 aa, Artificial Sequence
  Synthetic Construct
  VARIANT 1: Xaa = beta-A or S
XWWGSWATPRRRWWR

SEQ ID NO: 52 - protein, 15 aa, Artificial Sequence
  Synthetic Construct
  VARIANT 1: Xaa = beta-A or S
XWWRWWAPWARSWWR

SEQ ID NO: 53 - protein, 20 aa, Artificial Sequence
  Synthetic Construct
  VARIANT 1: Xaa = Any Amino Acid or none
  VARIANT 6, 7, 8, 15, 16, 17, 18: Xaa = Any Amino Acid
XKWRSXXXRWRLWRXXXXSR

SEQ ID NO: 54 - protein, 20 aa, Artificial Sequence
  Synthetic Construct
  VARIANT 1: Xaa = beta-A, S or none
  VARIANT 6: Xaa = A or V
  VARIANT 7: Xaa = G or L
  VARIANT 8: Xaa = W or Y
  VARIANT 15: Xaa = V or S
  VARIANT 16: Xaa = R, V, or A
  VARIANT 17: Xaa = S or L
  VARIANT 18: Xaa = W or Y
XKWRSXXXRWRLWRXXXXSR

SEQ ID NO: 55 - protein, 19 aa, Artificial Sequence
  Synthetic Construct
KWRSAGWRWRLWRVRSWSR

SEQ ID NO: 56 - protein, 19 aa, Artificial Sequence
  Synthetic Construct
KWRSALYRWRLWRVRSWSR

SEQ ID NO: 57 - protein, 19 aa, Artificial Sequence
  Synthetic Construct
KWRSALYRWRLWRSRSWSR

SEQ ID NO: 58 - protein, 19 aa, Artificial Sequence
  Synthetic Construct
KWRSALYRWRLWRSALYSR

SEQ ID NO: 59 - protein, 21 aa, Artificial Sequence
  Synthetic Construct
KWRSSAGWRSWRLWRVRSWSR

SEQ ID NO: 60 - protein, 21 aa, Artificial Sequence
  Synthetic Construct
KWRSSAGWRWRSLWRVRSWSR

SEQ ID NO: 61 - protein, 21 aa, Artificial Sequence
  Synthetic Construct
KWRSAGWRSWRLWRVRSSWSR

SEQ ID NO: 62 - protein, 21 aa, Artificial Sequence
  Synthetic Construct
KWRSSALYRSWRLWRSRSWSR

SEQ ID NO: 63 - protein, 21 aa, Artificial Sequence
  Synthetic Construct
KWRSSALYRWRSLWRSRSWSR

SEQ ID NO: 64 - protein, 21 aa, Artificial Sequence
  Synthetic Construct
KWRSALYRSWRLWRSRSSWSR

SEQ ID NO: 65 - protein, 21 aa, Artificial Sequence
  Synthetic Construct
KWRSALYRWRSLWRSSRSWSR

SEQ ID NO: 66 - protein, 21 aa, Artificial Sequence
  Synthetic Construct
KWRSALYRWRLWRSSRSWSSR

SEQ ID NO: 67 - protein, 21 aa, Artificial Sequence
  Synthetic Construct
KWRSSALYRWRSLWRSALYSR

SEQ ID NO: 68 - protein, 21 aa, Artificial Sequence
  Synthetic Construct
KWRSSALYRSWRLWRSALYSR

SEQ ID NO: 69 - protein, 21 aa, Artificial Sequence
  Synthetic Construct
KWRSALYRWRSLWRSSALYSR

SEQ ID NO: 70 - protein, 21 aa, Artificial Sequence
  Synthetic Construct
KWRSALYRWRLWRSSALYSSR

SEQ ID NO: 71 - protein, 21 aa, Artificial Sequence
  Synthetic Construct
KETWWETWWTEWSQPKKKRKV

SEQ ID NO: 72 - protein, 21 aa, Artificial Sequence
  Synthetic Construct
KETWFETWFTEWSQPKKKRKV

SEQ ID NO: 73 - protein, 15 aa, Artificial Sequence
  Synthetic Construct
KWFETWFTEWPKKRK

SEQ ID NO: 74 - protein, 27 aa, Artificial Sequence
  Synthetic Construct
GALFLGFLGAAGSTMGAWSQPKKKRKV

SEQ ID NO: 75 - protein, 16 aa, Artificial Sequence
  Synthetic Construct
AKWFERWFREWPRKRR

SEQ ID NO: 76 - protein, 16 aa, Artificial Sequence
  Synthetic Construct
AKWWERWWREWPRKRR

SEQ ID NO: 77 - protein, 20 aa, Artificial Sequence
  Synthetic Construct
ALWRALWRLWRSLWRLLWKA

SEQ ID NO: 78 - protein, 20 aa, Artificial Sequence
  Synthetic Construct
ALRWWLRWASRWFSRWAWWR

SEQ ID NO: 79 - protein, 20 aa, Artificial Sequence
  Synthetic Construct
AKWRSAGWRWRLWRVRSWSR

SEQ ID NO: 80 - protein, 20 aa, Artificial Sequence
  Synthetic Construct
AKWRSALYRWRLWRVRSWSR

SEQ ID NO: 81 - protein, 20 aa, Artificial Sequence
  Synthetic Construct
GLWRALWRLLRSLWRLLWKV

SEQ ID NO: 82 - protein, 17 aa, Artificial Sequence
  Synthetic Construct
RQIKIWFQNRRMKWKKC

SEQ ID NO: 83 - protein, 31 aa, Artificial Sequence
  Synthetic Construct
CRRRQRRKKRGGDIMGEWGNEIFGAIAGFLG

SEQ ID NO: 84 - protein, 27 aa, Artificial Sequence
  Synthetic Construct
KKALLALALHHLAHLALHLALALKKAC

SEQ ID NO: 85 - protein, 4 aa, Artificial Sequence
  Synthetic Construct
GYVS

SEQ ID NO: 86 - protein, 4 aa, Artificial Sequence
  Synthetic Construct
YVSK

SEQ ID NO: 87 - protein, 4 aa, Artificial Sequence
  Synthetic Construct
YIGS

SEQ ID NO: 88 - protein, 5 aa, Artificial Sequence
  Synthetic Construct
GYVSK

SEQ ID NO: 89 - protein, 5 aa, Artificial Sequence
  Synthetic Construct
YIGSR

SEQ ID NO: 90 - protein, 5 aa, Artificial Sequence
  Synthetic Construct
GGGGS

SEQ ID NO: 91 - protein, 5 aa, Artificial Sequence
  Synthetic Construct
SGGGG

SEQ ID NO: 92 - protein, 29 aa, Artificial Sequence
  Synthetic Construct
GYVSKGGGGSKWRSAGWRWRLWRVRSWSR

SEQ ID NO: 93 - protein, 29 aa, Artificial Sequence
  Synthetic Construct
KWRSAGWRWRLWRVRSWSRSGGGGGYVSK

SEQ ID NO: 94 - protein, 26 aa, Artificial Sequence
  Synthetic Construct
GYVSKGAKWRSAGWRWRLWRVRSWSR

SEQ ID NO: 95 - protein, 31 aa, Artificial Sequence
  Synthetic Construct
GYVSKGGGGSKETWWETWWTEWSQPKKKRKV

SEQ ID NO: 96 - protein, 30 aa, Artificial Sequence
  Synthetic Construct
KETWWETWWTEWSQPKKKRKVGGGGGYVSK

SEQ ID NO: 97 - protein, 22 aa, Artificial Sequence
  Synthetic Construct
AKETWWETWWTEWSQPKKKRKV

SEQ ID NO: 98 - protein, 25 aa, Artificial Sequence
  Synthetic Construct
GGGGKETWWETWWTEWSQPKKKRKV

SEQ ID NO: 99 - protein, 4 aa, Artificial Sequence
  Synthetic Construct
IGSR